(19) United States
(12) Patent Application Publication
(10) Pub. No.: US 2011/0256072 A1
(43) Pub. Date: Oct. 20, 2011

(54) FOOD AND COMPOSITION EACH COMPRISING PHOSPHORYLATED SACCHARIDE, POLYPHENOL AND FLUORIDE

(75) Inventors: Tomoko Tanaka, Osaka (JP); Hiroshi Kamasaka, Osaka (JP)

(73) Assignee: EZAKI GLICO CO., LTD., Osaka (JP)

(21) Appl. No.: 13/131,735

(22) PCT Filed: Nov. 27, 2009
(86) PCT No.: PCT/JP2009/070063

§ 371 Date: Jun. 21, 2011

(30) Foreign Application Priority Data
Nov. 28, 2008 (JP) 2008-305720

(51) Int. Cl. A61K 8/69; A61Q 11/00; A61K 9/68

(52) U.S. Cl. 424/48; 424/52

(57) ABSTRACT

A cariostatic food is provided. The food comprises: (1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt; (2) a fluoride; and (3) a polyphenol; wherein the phosphorylated saccharide is composed of a saccharide moiety and phosphate group(s); the content of the component (1) in the food is in an amount suitable to make the calcium concentration in saliva in the oral cavity to be 1 mM to 12 mM when the food exists in the oral cavity; and the content of fluoride in the food is in an amount suitable to make the fluoride concentration in saliva in the oral cavity to be 0.2 ppm to 100 ppm when the food exists in the oral cavity.

### TECHNICAL FIELD

The present invention relates to a cariostatic oral composition and food, which contain fluoride, a low-concentration polyphenol, and a phosphorylated saccharide or a salt thereof (for example, a phosphorylated saccharide calcium salt). More particularly, the present invention relates to a cariostatic oral composition and food, which would reduce the generation of dental caries by remineralization of teeth or the like.

### BACKGROUND ART

Dental caries refers to a parenchymatous defect which occurs as a result of demineralization of the tooth caused by an organic acid produced due to oral bacteria existing on a tooth surface, and is generally known as tooth decay. It has recently been found that a phenomenon called early caries lesion arises before the occurrence of dental caries. Early caries lesion refers to a state where parenchymatous defect of the tooth has not arisen and a tooth surface layer is retained, while calcium and phosphoric acid have been lost from the subsurface layer of a tooth surface. When early caries lesion arises, loss of calcium and phosphoric acid causes a change in a crystalline state of the tooth and thus the tooth surface looks white. Since dental caries is a parenchymatous defect, natural restoration is impossible and it is impossible to fill the defective part unless undergoing treatment by a dentist. On the contrary, in the case of early caries lesion, natural restoration is possible although it requires a long period of time. This is because the phenomena of demineralization and remineralization of the tooth usually arises in the oral cavity.

In general, diffusion of organic acid produced by oral bacteria existing on a tooth surface is obstructed by some obstacles and thus the tooth is exposed to high-concentrations of organic acid, resulting in the formation of dental caries. In this sense, all oral bacteria having sugar fermentation abilities which produces organic acid through metabolism can cause dental caries. Substrates, which are useful for the production of the organic acid, are saccharides and include monosaccharides and oligosaccharides, such as glucose and sucrose; and polysaccharides, such as a starch which is a polymer of a monosaccharide.

Factors, causing obstructing diffusion of the organic acid, are roughly classified into the following two factors: (1) starch taken during a meal remains on a tooth cervix part and a tooth root part; and (2) insoluble glucan produced by bacteria, using easily decomposable saccharides such as sucrose (i.e. fermentable saccharides) as a substrate, adhering to a tooth surface.

Regarding the above-described factor (1), all bacteria existing in the oral cavity with sugar fermentation abilities, such as lactobacillus, are considered to be one of the causative bacteria. In this case, it is known that dental caries will generally proceed slowly.

The above-described factor (2) is considered to be a principal cause of current dental caries. As the causative bacteria of the factor (2), Streptococcus mutans and Streptococcus sobrinus are considered. These bacteria are streptococci in the form of spherical bacteria each having a diameter of about 0.6 μm that are connected in moniliform. These bacteria actively produce a water-insoluble α-glucan in the presence of sucrose. This glucan has a property of very strongly adhering to the surface of a tooth. These bacteria exhibit an acid-producing ability by quick metabolism of sucrose. These bacteria have strong acid resistance and therefore can survive even under an acidic condition where other bacteria cannot grow. A water-insoluble α-glucan has strong stickiness and therefore can firmly bond bacteria to the surface of a tooth and the like. Diffusion of an organic acid produced by bacteria is obstructed by the water-insoluble glucan adhered to a tooth surface. As a result, a high-concentration of organic acid is accumulated on a tooth surface and thus the tooth surface is exposed to the high-concentration organic acid. In this case, dental caries proceeds quickly as compared to the case of factor (1).

There has been a new approach developed for prevention of dental caries at a microscopic level, e.g. demineralization and remineralization of the tooth, in the study of tooth health (Non-Patent Document 1). Teeth are composed of a dentin portion and an enamel portion, and the dentin is coated with the enamel. About 97% of the enamel is constituted from hydroxyapatite [Ca₁₀(PO₄)₆(OH)₂]. Hydroxyapatite is a crystal structure composed mainly of calcium and phosphoric acid. The enamel is the hardest portion of a tooth and prevents important calcium or phosphoric acid from eluting (demineralization) from the inside of the enamel due to organic acid produced by bacteria in dental plaque, acid contained in food and the like. The organic acid penetrates into the enamel from the spaces between enamel prisms filled with moisture and dissolves hydroxyapatite by a process called demineralization. This loss of calcium and phosphoric acid from the enamel tissue, as a result, causes early caries lesion at the subsurface of the enamel. early caries lesion can be restored. That is, calcium and phosphate ions penetrate into the subsurface dental caries portion and the lost apatite can be restored by a process called remineralization.

By taking meals or snacks, plaque (dental plaque) is formed in the oral cavity and organic acid is produced, thus leading to a decrease in pH and dissolution of the enamel. This phenomenon is demineralization. The case where a demineralized portion remains at the subsurface and tooth surface remains is early caries lesion, whereas when demineralization proceeds to cause a tooth surface to cave in, carious cavity is formed, thus leading to dental caries. The pH of dental plaque becomes acidic every time food and beverages containing fermentable carbohydrate are taken, and the critical pH is exceeded and demineralization is initiated. This is caused by a role of acid producing bacteria in dental plaque. In addition to dental caries bacteria, various factors such as dentition of teeth, and aging are involved in dental caries. Acidic food and beverages also increase the risk of dental caries. Demineralization due to acidic food and beverages is non-bacterial and is called tooth erosion. Tooth erosion refers to a phenomenon in which a surface of a tooth is chemically dissolved by an acid or chelation without involvement of dental caries bacteria. Recently, intense interest has been shown towards tooth erosion of mainly infants and young people due to carbonated beverages and sports beverages; tooth erosion of mainly adults and old people due to alcoholic beverages or healthy beverages, and tooth fracture and tooth attrition associated with tooth erosion. The critical pH of enamel is 5.5 and the pH value of many commercially available beverages is lower than 5.5. Usually, saliva has a cleaning effect of flushing out stains on the surface of a tooth and an acid buffering effect which neutralizes acid. The enamel is protected by these two functions. However, when beverages having low pH values are taken frequently and excessively, these effects cannot be sufficiently exerted. Moreover, the amount of saliva secreted decreases during sleep. Therefore, when one sleeps without sufficient washing of the oral cavity after drinking beverages having low pH values, the period during which the oral cavity is exposed to an acidic environment increases and tooth erosion occurs easily.

On the other hand, when the pH in the oral cavity returns to neutral through an increase in the pH due to receiving buffering action from saliva or the like at the stage of early caries lesion, and calcium ions and phosphate ions are supplied, the enamel is formed again. This phenomenon is remineralization.

Therefore, the followings are considered to be important as measures of preventing and treating dental caries: to prevent becoming a nutrient source of oral bacteria which is a cause of dental caries and the production of an organic acid; to prevent becoming a nutrient source of Streptococcus mutans which is a cause of dental caries and the generation of a water-insoluble glucan and an organic acid; to prevent a decrease in pH due to this organic acid by preventing the pH from exceeding the pH at which demineralization initiates (for example, to have a buffering action to prevent a decrease in pH); and to accelerate remineralization and the like.

There have been various studies done on an oral composition, food and the like for the treatment of early caries lesion utilizing remineralization.

Fluoride is used as a tooth strength improving material in pharmaceuticals and quasi-drugs. However, fluoride used in pharmaceuticals and quasi-drugs is usually a compound such as sodium fluoride or sodium silicofluoride, and cannot be utilized for food in JAPAN.

In the dental field, the effect of fluoride is generally known. For example, it is known that when a portion or all of hydroxyl groups (—OH) of a hydroxyapatite is substituted with fluorine (—F) to obtain a fluorapatite or fluoroxyapatite, the apatite becomes harder. This phenomenon is useful for improving hardness of a tooth. It is also possible to avoid an influence of an acid by adsorbing fluoride ions around a crystal, thereby coating an enamel surface through a fluoride coating-like ion coating action. This is useful for obtaining acid resistance.

In the prevention of tooth decay with fluoride, it is important to completely surround (coat) an enamel surface with fluoride ions. In the case of incomplete coating, demineralization of a tooth initiates from a site where the enamel surface is not coated with fluoride ions, in an acidic state.

Fluoride compounds, which are now utilized for the prevention of dental caries, are listed in the following Table 1A.

TABLE 1A

| | | | Fluorine | | Inhibition |
|---|---|---|---|---|---|
| | Method | Kind of fluoride | concentration | subject Tooth | rate |
| Systemic | Addition of | Sodium silicofluoride | 0.6-1 ppm | Permanent tooth | 40-60% |
| application | fluoride to city | Hexafluorosilicic acid | | Baby tooth | about |
| method | water | Sodium fluoride | | | 30% |
| | | Ammonium hexafluorosilicate | | | |
| | | Calcium fluoride | | | |
| | Addition of | Sodium fluoride | 200 mg NaF | | 25-50% |
| | fluoride to | | per 1 Kg of | | |
| | common salt | | common salt | | |
| | Taking fluoride | Sodium fluoride | 0.5-1.0 mg | | 20-40% |
| | tablets | | F/day | | |
| Local | Fluoride | 2% Sodium fluoride solution | 9,000 ppm | Permanent tooth | 20-40% |
| application | application | | 12,300 ppm | Permanent tooth | 20-50% |
| method | method to dental | phosphoric acid-Acidic | 9,000 ppm | | |
| | surface | fluoride solution | 19,400 ppm | Permanent tooth | 20-50% |
| | | SnF₂: 8% Solution | 9,700 ppm | | |
| | | SnF₂: 4% Solution | | | |
| | Fluoride mouth | NaF: 0.05% every day method | 226 ppm | Permanent tooth | 20-50% |
| | washing method | NaF: 0.2% every week method | 900 ppm | | |
| | Fluoride added | Monofluorophosphate | 1000 ppm | | 15-20% |
| | dentifrice | (for example, Na₂PO₃F) | | | |
| | | NaF | | | |
| | | SnF₂ | | | |

(Excerpt: Fukkabutsu rinsho oyou no science (Science of Clinical Application of Fluorides), supervised by Yoshinori TAKAESU

As described above, the concentration of fluoride used in a commonly utilized local application method is of a very high concentration, about 200 ppm or more. The concentration of fluoride to be added to city water as described in the systemic application method is from 0.6 to 1 ppm and is lower than those in the local application method. However, the addition of fluoride to city water is not approved in Japan.

In the case of using fluoride in food, there are various problems. First, there is a problem that sodium fluoride (NaF), calcium fluoride (CaF) and tin fluoride (SnF₂), which have conventionally been used as a fluoride material, are quasi-drugs and are therefore materials which cannot be used in food. Regarding fluoride materials which can be utilized in food, tea extract, fishery products, vegetables (for example, root crops (for example, tubers)), cereals, coffee, deep-ocean water, and the like are known to contain a large amount of fluoride. Particularly, it is known that tea extract has high fluoride content. However, although tea extract has a high fluoride content, it also has a high polyphenol content and therefore inhibits the remineralization effect as described in Comparative Tests 1 to 2 of the present application. Therefore, it is impossible to utilize a conventional tea extract for remineralization. For example, Non-Patent Document 1 describes acid resistance of a green tea extract-added gum, but indicated in Table 3 that there is no significant difference in the amount of mineral loss between a green tea extract-added gum and a placebo gum. That is, the publication indicates that the green tea extract is not effective for remineralization.

For example, Patent Document 1 discloses a composition for food and beverages and an oral composition, which has a cariostatic function, and which contains a buffering agent such as phosphorylated oligosaccharide. Patent Document 1 describes in paragraph 0107 that the composition for food and beverages and the oral composition can further contain fluoride, if necessary. Patent Document 1 describes that the composition contains fluoride in an amount which does not exceed 1,000 ppm, preferably from 0.1 to 500 ppm, and more preferably from 0.1 to 300 ppm.

Patent Document 2 (Japanese Laid-open Patent Publication No. 8-104696) describes in paragraph 0001 that a phosphorylated saccharide has a tooth decay prevention effect and a phosphorylated saccharide can be added to food, beverages and samples as well as oral compositions such as tooth paste, mouth wash and troches. Patent Document 2 does not mention about fluoride.

Patent Document 3 (Japanese Patent Gazette No. 3,333,584) relates to a composition for enhancing acid resistance of tooth. Patent Document 3 discloses a composition for enhancing acid resistance of tooth, characterized in that the composition comprises 10 to 2,000 ppm of a tea polyphenol, 20 to 1,000 ppm of fluoride as a fluoride, and 50 to 1,000 ppm of an aluminum salt.

Heretofore, a fluoride agent and a calcium agent could not have been used in combination. This is because reactivity between calcium and fluoride is high and thus these are precipitated as calcium fluoride (CaF₂) before reaching the target site.

Various techniques are disclosed about the use of a tea extract for the enhancement of the tooth strength. For example, Patent Document 4 (Japanese Laid-open Patent Publication No. 2005-29496) discloses a tooth strength-enhancing composition, an oral composition, and a food and a beverage, characterized in that tea extract having decreased content of polyphenols while comprising minerals as an active ingredient. Patent Document 4 relates to a tooth strength-enhancing composition characterized in that tea extract having decreased content of polyphenols while comprising minerals is an active ingredient. The object of the Patent Document 4 is improving the quality of taste and Patent Document 4 does not mention about fluoride. Patent Document 4 describes, in paragraph 0014, such a hypothesis that a composition other than polyphenols contained in tea extract (for example, a mineral) is significantly involved in tooth strength enhancement and the tooth strength enhancing effect is remarkably exerted by decreasing polyphenols which act as an inhibiting factor to the composition other than the relevant polyphenols. Patent Document 4 further describes in paragraph [0016] that, in the tooth strength-enhancing composition, the proportion of the mineral contributing to tooth strength enhancement is relatively increased corresponding to the decrease in the proportion of polyphenols, and therefore the tooth strength enhancing effect is remarkably exerted by decreasing the proportion of polyphenols which have hitherto acted as an inhibiting factor. However, according to the description in paragraph 0025 of Patent Document 4, a “mineral-containing polyphenol-reduced tea extract” refers to one containing a mineral derived from tea, and specifically refers to one containing one, or two or more kinds of substances selected from the group consisting of potassium, calcium, phosphorus, sodium, manganese, magnesium, iron, copper and zinc. As described above, Patent Document 4 does not intend to include fluoride in a mineral-containing polyphenol-reduced tea extract.

However, even if these conventional methods are used, it is impossible to achieve complete remineralization of a demineralized portion of early caries lesion, and the effect is restrictive.

Therefore, a food and a composition for remineralization, which have more excellent effect as compared to that of prior art, are desired.

### PRIOR ART DOCUMENTS

### Patent Documents

Patent Document 1: Japanese Laid-open Patent Publication No. 2002-325557
Patent Document 2: Japanese Laid-open Patent Publication No. 8-104696
Patent Document 3: Japanese Patent Gazette No. 3,333,584
Patent Document 4: Japanese Laid-open Patent Publication No. 2005-29496

### Non-Patent Documents

Non-Patent Document 1: Toshiaki OHMORI et al., Journal of Nutritional Food, Vol. 7, No. 2, 2004, 31-41

### SUMMARY OF THE INVENTION

### Problems to be Solved by the Invention

The present invention is intended to solve the problems described above, and it is an object of the invention to provide a food and a composition, which contain a phosphorylated saccharide, fluoride and a polyphenol.

### Means for Solving the Problems

The inventors of the present invention conducted intensive studies in order to solve the problems as described above, and as a result, they found that, when conventional tea extract is used, fluoride cannot be effectively utilized, however, a high remineralization effect and a high tooth improving effect can be obtained by using fluoride in the form of a low polyphenol content-tea extract in combination with POs-Ca. Thus, the inventors completed the present invention based on this finding.

The inventors of the present invention have found that polyphenol contained in a tea extract adsorbs a mineral to decrease its absorption rate and that low concentration of fluoride is preferable, as both the remineralization effect of lesion where early caries lesion has been formed and the tooth improving effect are not satisfactory when the fluoride concentration is too high. In the present invention, a food and an oral composition, which utilize this characteristic, are provided. In the present invention, a phosphorylated saccharide calcium salt (or a combination of a phosphorylated saccharide salt (excluding the calcium salt) and a water-soluble calcium salt)+low-concentration F+low polyphenol are important.

In the case where a high fluoride content-tea extract containing a low concentration of polyphenol is used in combination with a phosphorylated saccharide calcium salt (or a combination of a phosphorylated saccharide salt (excluding the calcium salt) and a water-soluble calcium salt), a high tooth improving effect and a remineralization effect can be obtained in a lower fluoride concentration than that conventionally assumed. It was found that, to the contrary, when the fluoride concentration is high, both the tooth improving effect and the remineralization effect are inhibited.

In order to achieve the object described above, the present invention provides, for example, the following means:

(Item 1) A cariostatic food comprising:

(1) (i) a phosphorylated saccharide calcium salt; or

(ii) a combination of

a phosphorylated saccharide salt other than a phosphorylated saccharide calcium salt or

a phosphorylated saccharide, and

a water-soluble calcium salt other than a phosphorylated saccharide calcium salt;

(2) a fluoride; and

(3) a polyphenol; wherein

the phosphorylated saccharide is composed of a saccharide moiety and phosphate group(s);

the content of the component (1) in the food is in an amount suitable to make the calcium concentration in saliva in the oral cavity to be 1 mM to 12 mM when the food exists in the oral cavity;

the content of the fluoride in the food is in an amount suitable to make the fluoride concentration in saliva in the oral cavity to be 0.2 ppm to 100 ppm when the food exists in the oral cavity;

the content of the polyphenol in the food is in an amount suitable to make the polyphenol concentration in saliva in the oral cavity to be 0.001% by weight to 0.1% by weight when the food exists in the oral cavity; and

the food remains in the oral cavity for 5 minutes or more upon eating.

(Item 2) A cariostatic food comprising:

(1) (i) a phosphorylated saccharide calcium salt; or

(ii) a combination of

a phosphorylated saccharide salt other than a phosphorylated saccharide calcium salt or

a phosphorylated saccharide, and

a water-soluble calcium salt other than a phosphorylated saccharide calcium salt;

(2) a fluoride; and

(3) a polyphenol; wherein

the phosphorylated saccharide is composed of a saccharide moiety and phosphate group(s);

the content of the component (1) in the food is in an amount suitable to make the calcium concentration in saliva in the oral cavity to be 1 mM to 12 mM when the food exists in the oral cavity;

the content of the fluoride in the food is in an amount suitable to make the fluoride concentration in saliva in the oral cavity to be 0.2 ppm to 100 ppm when the food exists in the oral cavity;

the content of the polyphenol in the food is in an amount suitable to make the polyphenol concentration in saliva in the oral cavity in to be 10 times to 200 times the fluoride concentration when the food exists in the oral cavity; and

the food remains in the oral cavity for 5 minutes or more upon eating.

(Item 3) A cariostatic food comprising:

(1) (i) a phosphorylated saccharide calcium salt; or

(ii) a combination of

a phosphorylated saccharide salt other than a phosphorylated saccharide calcium salt or

a phosphorylated saccharide, and

a water-soluble calcium salt other than a phosphorylated saccharide calcium salt;

(2) a fluoride; and

(3) a polyphenol; wherein

the phosphorylated saccharide is composed of a saccharide moiety and phosphate group(s);

the content of the component (1) in the food is in an amount suitable to make the calcium concentration in saliva in the oral cavity to be 1 mM to 12 mM when the food exists in the oral cavity;

the content of the fluoride in the food is in an amount suitable to make the fluoride concentration in saliva in the oral cavity to be 0.005 times to 0.1 times the concentration of calcium derived from the component (1) when the food exists in the oral cavity;

the content of the polyphenol in the food is in an amount suitable to make the polyphenol concentration in saliva in the oral cavity to be 10 times to 200 times the fluoride concentration when the food exists in the oral cavity; and

the food remains in the oral cavity for 5 minutes or more upon eating.

(Item 4) The food according to any one of Items 1 to 3, which is a chewing gum, a candy, tablet candy or a frozen dessert.

(Item 5) The food according to any one of Items 1 to 4, wherein the polyphenol is a tea polyphenol.

(Item 6) The food according to any one of Items 1 to 5, wherein the saccharide moiety is a glucan or a reduced glucan.

(Item 7) The food according to Item 6, wherein the degree of polymerization of the saccharide moiety is 2 to 8.

(Item 8) The food according to Item 7, wherein the number of the phosphate group(s) is from 1 to 2.

(Item 9) The food according to any one of Items 1 to 8, wherein the component (1) is a phosphorylated saccharide calcium salt.

(Item 10) The food according to any one of Items 1 to 9, which further contains a phosphoric acid source compound.

(Item 11) The food according to Item 10, wherein the phosphoric acid source compound is selected from the group consisting of phosphoric acid, sodium phosphate, potassium phosphate, polyphosphoric acid and a cyclic phosphate.

(Item 12) The food according to Item 10 or 11, wherein the concentration of the phosphoric acid source compound is 9 mM or less.

(Item 13) The food according to any one of Items 1 to 12, wherein the content of the polyphenol in the food is in an amount suitable to make the polyphenol amount in saliva in the oral cavity to be 0.001% by weight to 0.02% by weight when the food exists in the oral cavity.

(Item 14) The food according to any one of Items 1 to 13, wherein the content of the fluoride in the food is in an amount suitable to make the fluoride concentration in saliva in the oral cavity to be 0.2 ppm to 1 ppm when the food exists in the oral cavity.

(Item 15) A cariostatic oral composition comprising:

(1) (i) a phosphorylated saccharide calcium salt; or

(ii) a combination of

a phosphorylated saccharide salt other than a phosphorylated saccharide calcium salt or

a phosphorylated saccharide, and

a water-soluble calcium salt other than a phosphorylated saccharide calcium salt;

(2) a fluoride; and

(3) a polyphenol; wherein

the phosphorylated saccharide is composed of a saccharide moiety and phosphate group(s);

the concentration of the component (1) is 1 mM to 12 mM in terms of the concentration of calcium;

the content of the fluoride in the composition is in an amount suitable to make the fluoride concentration in the mixture of the composition and saliva in the oral cavity to be 0.2 ppm to 100 ppm when the composition is used in the oral cavity;

the content of the polyphenol in the composition is in an amount suitable to make the polyphenol concentration in the mixture of the composition and saliva in the oral cavity in to be 0.001% by weight to 0.1% by weight when the composition is used in the oral cavity; and

the composition remains in the oral cavity for 5 minutes or more.

(Item 16) A cariostatic oral composition comprising:

(1) (i) a phosphorylated saccharide calcium salt; or

(ii) a combination of a phosphorylated saccharide salt other than a phosphorylated saccharide calcium salt or a phosphorylated saccharide, and a water-soluble calcium salt other than a phosphorylated saccharide calcium salt;

(2) a fluoride; and

(3) a polyphenol; wherein

the phosphorylated saccharide is composed of a saccharide moiety and phosphate group(s);

the content of the component (1) in the composition is in an amount suitable to make the calcium concentration in the mixture of the composition and saliva in the oral cavity to be 1 mM to 12 mM when the composition is used in the oral cavity:

the content of the fluoride in the composition is in an amount suitable to make the fluoride concentration in the mixture of the composition and saliva in the oral cavity to be 0.2 ppm to 100 ppm when the composition is used in the oral cavity;

the content of the polyphenol in the composition is in an amount suitable to make the polyphenol concentration in the mixture of the composition and saliva in the oral cavity to be 10 times to 200 times larger than the fluoride concentration when the composition is used in the oral cavity; and

the composition remains in the oral cavity for 5 minutes or more.

(Item 17) A cariostatic oral composition comprising:

(1) (i) a phosphorylated saccharide calcium salt; or

(ii) a combination of a phosphorylated saccharide salt other than a phosphorylated saccharide calcium salt or a phosphorylatedsaccharide, and a water-soluble calcium salt other than a phosphorylated saccharide calcium salt;

(2) a fluoride; and

(3) a polyphenol; wherein

the phosphorylated saccharide is composed of a saccharide moiety and phosphate group(s);

the content of the component (1) in the composition is in an amount suitable to make the calcium concentration in the mixture of the composition and saliva in the oral cavity to be 1 mM to 12 mM when the composition is used in the oral cavity:

the content of the fluoride in the composition is in an amount suitable to make the fluoride concentration in the mixture of the composition and saliva in the oral cavity to be 0.005 times to 0.1 times the concentration of calcium derived from the component (1) when the composition is used in the oral cavity;

the content of the polyphenol in the composition is in an amount suitable to make the polyphenol concentration in the mixture of the composition and saliva in the oral cavity to be 10 times to 200 times the fluoride concentration when the composition is used in the oral cavity; and

the composition remains in the oral cavity for 5 minutes or more upon eating.

(Item 18) The composition according to any one of Items 15 to 17, wherein the polyphenol is a tea polyphenol.

(Item 19) The composition according to any one of Items 15 to 18, wherein the saccharide moiety is a glucan or a reduced glucan.

(Item20) The composition according to Item 19, wherein the degree of polymerization of the saccharide moiety is 2 to 8.

(Item21) The composition according to Item20, wherein the number of the phosphate group is from 1 to 2.

(Item 22) The composition according to any one of Items 15 to 21, wherein the component (1) is a phosphorylated saccharide calcium salt.

(Item 23) The composition according to any one of Items 15 to 22, which further contains a phosphoric acid source compound.

(Item 24) The composition according to Item23, wherein the phosphoric acid source compound is selected from the group consisting of phosphoric acid, sodium phosphate, potassium phosphate, polyphosphoric acid and a cyclic phosphate.

(Item 25) The composition according to Item 23 or 24,wherein the content of the phosphoric acid source compound in the composition is in an amount suitable to make the phosphoric acid concentration in the mixture of the composition and saliva in the oral cavity to be 9 mM or less when the composition is used in the oral cavity.

(Item 26) The composition according to any one of Items 15 to 25, wherein the content of the polyphenol in the composition is in an amount suitable to make the polyphenol concentration in the mixture of the composition and saliva in the oral cavity to be 0.001% by weight to 0.02% by weight when the composition is used in the oral cavity.

(Item 27) The composition according to any one of Items 15 to 26, wherein the content of the fluoride in the composition is in an amount suitable to make the fluoride concentration in the mixture of the composition and saliva in the oral cavity to be 0.2 ppm to 1 ppm when the composition is used in the oral cavity.

(Item 28) The composition according to any one of Items 15 to 27, which is used for the treatment of early caries lesion.

(Item 29) The composition according to any one of Items 15 to 27, which is used for the enhancement of tooth strength of a healthy human.

(Item 30) The composition according to any one of Items 15 to 27, which is a dentifrice, a mouth rinsing agent, a troche, a gel, a spray, a paste, a liniment or an ointment.

(Item 31) The oral composition according to Item 27, which is a dentifrice or a mouth rinsing agent.

(Item 32) A method for producing the food according to Item 1, which comprises the steps of adding:

(1) (i) a phosphorylated saccharide calcium salt; or

(ii) a combination of

a phosphorylated saccharide salt other than a phosphorylated saccharide calcium salt or

a phosphorylated saccharide, and

a water-soluble calcium salt other than a phosphorylated saccharide calcium salt; and

(2) a tea extract,

to a food raw material, wherein

the tea extract contains a fluoride and a polyphenol, and the ratio of the concentration of fluoride to that of the polyphenol in the tea extract is fluoride: polyphenol=1:10 to 1:200.

(Item 33) A method for producing the oral composition according to Item 13, which comprises the steps of adding:

(1) (i) a phosphorylated saccharide calcium salt; or

(ii) a combination of

a phosphorylated saccharide salt other than a phosphorylated saccharide calcium salt or

a phosphorylated saccharide, and

a water-soluble calcium salt other than a phosphorylated saccharide calcium salt; and

(2) a tea extract,

to an oral composition raw material, wherein

the tea extract contains a fluoride and a polyphenol, and the ratio of the concentration of fluoride to that of the polyphenol in the tea extract is fluoride: polyphenol=1:10 to 1:200.

### Effects of the Invention

By using a low polyphenol-high fluoride-containing tea extract and a phosphorylated saccharide calcium salt (or a combination of a phosphorylated saccharide salt (excluding the calcium salt) and a water-soluble calcium salt) in combination, a non-conventionally excellent remineralization effect can be obtained. Particularly, when the polyphenol content is decreased by removing most of the polyphenols from a conventional tea extract, fluoride in the extract can be effectively utilized for the improvement of acid resistance and remineralization.

By combining fluoride as a low polyphenol-high fluoride-containing tea extract, a polyphenol and a phosphorylated saccharide calcium salt (or a combination of a phosphorylated saccharide salt (excluding the calcium salt) and a water-soluble calcium salt), a high remineralization effect and a high tooth improving effect were obtained. The present invention provides an oral composition and food, which utilizes this characteristic.

By using fluoride, a polyphenol and a phosphorylated saccharide calcium salt (or a combination of a phosphorylated saccharide salt (excluding a calcium salt) and a water-soluble calcium salt) in combination, a tooth erosion preventive effect can be obtained.

Even when the low polyphenol-high fluoride-containing tea extract is added, bitterness, astringent taste and salty taste are scarcely exhibited. Furthermore, by combining POs-Ca with the low polyphenol-high fluoride-containing tea extract, bitterness, astringent taste and salty taste of POs-Ca can be relieved.

When an ionized substance of a phosphorylated saccharide and calcium ions coexist, remineralization is accelerated by enamel-specific supply of calcium in the oral cavity. In other words, when an ionized substance of a phosphorylated saccharide exists, calcium ions maintain solubility without being insolubilized by bonding with inorganic phosphoric acid under a neutral condition. Under normal environments (under environments where no hydroxyapatite exists), it does not release calcium and, when it reaches a place where hydroxyapatite exists, it releases calcium. Therefore, when a phosphorylated saccharide and calcium ions exist, a large amount of calcium is provided to hydroxyapatite and thus remineralization is remarkably accelerated. That is, the phosphorylated saccharide satisfies the following two points which are required for a remineralization accelerating substance in early caries lesion:

(1) to prevent insolubilization of calcium-phosphoric acid under a neutral pH condition; and
(2) to make calcium ions and phosphate ions reach the affected part and then subject them to remineralization.

Furthermore, it was found that the existence of a small amount of a polyphenol enables a further improvement in the remineralization effect. This action is quite contrary to an action of inhibiting the remineralization effect by the existence of a high-concentration polyphenol, and thus surprising.

Therefore, a combination of a phosphorylated saccharide, calcium ions, a low concentration polyphenol and fluoride exerts an excellent calcium providing effect, which is remarkably different to that of a conventional calcium compound, on hydroxyapatite.

### BRIEF DESCRIPTION OF THE DRAWINGS

FIG. 1 is a graph showing the results of Comparative Test 1-1. This graph shows a change over time in the pH and the percentage of soluble calcium in a remineralization solution containing no tea extract and containing phosphorylated oligosaccharide calcium. The symbol “black diamond” denotes the percentage of soluble calcium (%), and the symbol “white circle” denotes the pH. The right vertical axis denotes the percentage of soluble calcium, while the left vertical axis denotes the pH. The transverse axis denotes the time (minute). The symbol “black triangle” and the symbol “white triangle” denote a point in time of addition of crystal nuclei.

FIG. 2 is a graph showing the results of Comparative Test 1-2. This graph shows a change over time in the pH and the percentage of soluble calcium in a remineralization solution containing a conventional tea extract and phosphorylated oligosaccharide calcium. The symbol “white circle” denotes the percentage of soluble calcium (%), and the symbol “black diamond” denotes the pH. The right vertical axis denotes the percentage of soluble calcium, while the left vertical axis denotes the pH. The transverse axis denotes the time (minute). The symbol “black triangle” and the symbol “white triangle” denote a point in time of addition of the crystal nuclei.

FIG. 3 is a graph showing the results of Test 1. This graph shows a change over time in pH and the percentage of soluble calcium in a remineralization solution containing low polyphenol content-tea extract and phosphorylated oligosaccharide calcium. The symbol “black diamond” denotes the percentage of soluble calcium (%), and the symbol “white circle” denotes the pH. The right vertical axis denotes the percentage of soluble calcium, while the left vertical axis denotes the pH. The transverse axis denotes the time (minute). The symbol “blacktriangle” and the symbol “white triangle” denote a point in time of addition of crystal nuclei.

FIG. 4 shows the results of Example 1. The upper panels (a) to (c) of FIG. 4 show the results of an acid resistance test A, the middle panels (d) to (f) of FIG. 4 show the results of an acid resistance test B, and the lower panels (g) to (i) of FIG. 4 show the results of an acid resistance test C. In FIG. 4, (a), (d) and (g) show the results of X-ray photography of the portion subjected to only a demineralization treatment. In FIG. 4, (b), (e) and (h) show the results of X-ray photography of the portion subjected to first demineralization, then remineralization treatment, and which was not subsequently subjected to re-demineralization. In FIG. 4, (c), (f) and (i) show the results of X-ray photography of the portion subjected to first demineralization, then a remineralization treatment with various remineralization solutions, and then which was subsequently subjected to re-demineralization.

FIG. 5 is a graph showing the results of Tests 2-1 and 2-2, and Comparative Tests 2-1 and 2-2. This graph shows a change with time of the percentage of soluble calcium in remineralization solutions having various polyphenol contents. The symbol “large black circle” denotes the results when the polyphenol concentration is 0%. The symbol “small black circle” denotes the results when the polyphenol concentration is 0.011% by weight. The symbol “black diamond” denotes the results when the polyphenol concentration is 0.0017% by weight. The symbol “white square” denotes the results when the polyphenol concentration is 0.0022% by weight. The vertical axis denotes the percentage of soluble calcium, while the transverse axis denotes the time (minute). The symbol “black triangle” and the symbol “white triangle” denote a point in time of addition of crystal nuclei.

FIG. 6 shows a graph for a recovery rate of the amount of mineral loss (%). The vertical axis denotes a recovery rate of the amount of mineral loss (%).

FIG. 7 shows a recovery rate of the amount of mineral loss (%) for the case of only 0.5 ppm of fluoride, 0.5 ppm of fluoride+POs-Ca, or 0.5 ppm of fluoride+CaCl₂. The vertical axis denotes a recovery rate of the amount of mineral loss (%).

FIG. 8 shows a recovery rate of a lesion depth (%) for the case of only 0.5 ppm of fluoride, 0.5 ppm of fluoride+POs-Ca, or 0.5 ppm of fluoride+CaCl₂. The vertical axis denotes a recovery rate of a lesion depth (%).

FIG. 9 shows a hardness of a dental piece (ΔHV) of a demineralized portion and a remineralized portion for the cases of 0.5 ppm of fluoride+POs-Ca and 0.5 ppm of fluoride+CaCl₂. The vertical axis denotes the hardness (ΔHV; F/A). P0.5DEM denotes a demineralized site of 0.5 ppm of fluoride+POs-Ca (containing a polyphenol), P0.5REM denotes a remineralized site of 0.5 ppm of fluoride+POs-Ca (containing a polyphenol). C0.5DEM denotes a demineralized site of 0.5 ppm of fluoride+CaCl₂ (containing a polyphenol) and C0.5REM denotes a remineralized site of 0.5 ppm of fluoride+CaCl₂ (containing a polyphenol).

FIG. 10 shows a recovery rate of the amount of mineral loss due to remineralization.

FIG. 11 shows a recovery rate of a lesion depth due to remineralization.

FIG. 12 shows a recovery rate of the amount of mineral loss after re-demineralization.

FIG. 13 shows a recovery rate of a lesion depth after re-demineralization.

FIG. 14 shows a recovery rate of fluoride ions in the case of only tea fluoride (containing a polyphenol) and a recovery rate of fluoride ions in the case of containing tea fluoride and POs-Ca (containing a polyphenol), both the rates were obtained in Example 9.

FIG. 15 shows the concentration of calcium ions and the concentration of fluoride ions in saliva measured in Example 10. The symbol “white circle” denotes the concentration of calcium ions, and the symbol “black triangle” denotes the concentration of fluoride ions.

FIG. 16 shows the concentration of phosphate ions in saliva measured in Example 10.

FIG. 17 shows a Ca/P ratio in saliva measured in Example 10.

FIG. 18 shows an amount of saliva collected in Example 10.

FIG. 19 shows the pH in saliva measured in Example 10.

FIG. 20 shows a schematic diagram of a test cycle carried out in Example 11 and Comparative Example 11.

FIG. 21 shows a CLSM profile measured in Example 11 and Comparative Example 11. The symbol “black diamond” with a solid line denotes POs-Ca+F (containing a polyphenol), the symbol “black square” with a solid line denotes POs-Ca, the symbol “black square” with a broken line denotes F (containing a polyphenol), and the symbol “black triangle” with a broken line denotes a control.

FIG. 22 shows a surface roughness profile measured in Example 11 and Comparative Example 11. The symbol “black diamond” with a solid line denotes POs-Ca+F (containing a polyphenol), the symbol “black square” with a solid line denotes POs-Ca, the symbol “black square” with a broken line denotes F (containing a polyphenol), and the symbol “black triangle” with a broken line denotes a control.

FIG. 23 is a graph showing a change over time in the pH and the amount of soluble calcium (mM) in a remineralization solution. “(a)” shows the results of Example 12-1 using strontium fluoride as a fluoride agent, “(b)” shows the results of Example 12-2 using sodium monofluorophosphate as a fluoride agent, and “(c)” shows the results of Example 12-3 using potassium fluoride as a fluoride agent. The symbol “black circle” denotes an amount of soluble calcium (mM), and the symbol “black diamond” denotes the pH. The right vertical axis denotes the amount of soluble calcium (mM), while the left vertical axis denotes the pH. The transverse axis denotes the time (minute). The symbol “black triangle” denotes a point in time of addition of crystal nuclei.

FIG. 24 is a graph showing the evaluation results of bitterness.

FIG. 25 is a graph showing the evaluation results of astringent taste.

FIG. 26 is a graph showing the evaluation results of salty taste.

### MODE FOR CARRYING OUT THE INVENTION

Hereinafter, the present invention will be described in detail.

### 1. Definition

In the present description, a cariostatic function includes both a dental caries preventing function and a dental caries treating function. The dental caries treating function refers to a function of restoring a portion of a tooth which was lost due to dental caries. In the present description, “having a cariostatic function” means having one or more properties of the following: (1) to have a pH buffering action and an ability of inhibiting a decrease in pH due to acid produced by oral bacteria; (2) to have an ability of inhibiting the formation of an insoluble glucan produced by oral bacteria; and (3) to have an ability of accelerating remineralization of a tooth with early caries lesion. It is preferred to have two of the properties described above. It is most preferred to have all of the properties described above.

According to the composition and the food of the present invention, it is possible to stably provide phosphoric acid and calcium to the tooth damaged by dental caries. Since the tooth provided with phosphoric acid and calcium is remineralized, it is possible to restore a portion of the tooth lost due to dental caries.

Particularly, according to the present invention, since a buffering agent is added to the oral cavity, it is expected that a pH buffering action can be obtained in the oral cavity. By the pH buffering action in the oral cavity, phosphoric acid and calcium existing in saliva in the oral cavity are stably used for remineralization of a tooth. Therefore, it becomes possible to restore a tooth, which has conventionally been considered to be difficult or impossible.

Regarding demineralized lesion as an initial symptom of dental caries, when the conditions in the oral cavity are satisfactory, calcium and phosphoric acid are supplemented again (remineralized) to the demineralized enamel portion and the enamel portion is restored to a healthy state. In order to maintain a tooth in a healthy state, it is necessary that a mineral is supplied to the demineralized affected part by an action of saliva, thereby keeping the balance between demineralization and remineralization at a microscopic level. Generally, after eating and drinking, the pH in dental plaque tends to lower and an equilibrium relationship of “demineralization-remineralization” is lost. In the case where the relationship “demineralization>remineralization” is established, lesion proceeds. To the contrary, in the case where the relationship “remineralization>demineralization” is established, demineralized lesion gets better and the tooth is remineralized. The intraoral environment, particularly the pH, and the concentrations of calcium and phosphoric acid in saliva and dental plaque play an important role in the balance between demineralization and remineralization (Yoichi IIJIMA, Takashi KUMAGAI; Caries Control, Mechanism of Demineralization and Remineralization, Ishiyaku Pub, Inc.; 21-51, 1999). According to the present invention, since it is possible to adjust the intraoral environment to an environment where remineralization easily occurs, dental caries can be prevented, and also demineralized lesion as an initial symptom of dental caries can be treated, and thus teeth can be made healthy and strong.

### 2. Materials Used in the Present Invention

In the present invention, (1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt; (2) a fluoride; and (3) a polyphenol are used. If necessary, other materials can also be used.

(2a. Phosphorylated Saccharide and Phosphorylated Saccharide Salt)

The phosphorylated saccharide used in the present invention is composed of a saccharide moiety and phosphate group(s). As used in the present description, the term “phosphorylated saccharide” refers to a saccharide having at least one phosphate group(s) in the molecule. As used in the present description, the term “a phosphorylated saccharide salt” refers to a salt of a phosphorylated saccharide. As used in the present description, the term “phosphorylated saccharide inorganic salt” refers to an inorganic salt of a phosphorylated saccharide. As used in the present description, the term “a phosphorylated saccharide calcium salt” refers to a calcium salt of a phosphorylated saccharide.

The number of phosphate groups in the phosphorylated saccharide is not particularly limited and is preferably 10 or less, and more preferably 5 or less, per molecule of the phosphorylated saccharide. Furthermore, the number of phosphate groups in the phosphorylated saccharide is preferably 1, 2 or 3, and particularly preferably 1 or 2, per molecule of the phosphorylated saccharide.

The degree of polymerization of the saccharide moiety in the phosphorylated saccharide is preferably 2 or more, and more preferably 3 or more. The degree of polymerization of the saccharide in the phosphorylated saccharide is preferably about 100 or less, more preferably about 90 or less, more preferably about 80 or less, more preferably about 70 or less, more preferably about 60 or less, more preferably about 50 or less, more preferably about 40 or less, more preferably about 30 or less, more preferably about 20 or less, more preferably about 10 or less, more preferably about 9 or less, more preferably about 8 or less, more preferably about 7 or less, more preferably about 6 or less, and particularly preferably about 5 or less. It is noted that in the present description, those in which the degree of polymerization of the saccharide moiety in the phosphorylated saccharide is 10 or less is also referred to as a phosphorylated oligosaccharide.

The molecular weight of the phosphorylated saccharide is preferably about 400 or more, more preferably about 500 or more, furthermore preferably about 600 or more, and particularly preferably about 700 or more. The molecular weight of the phosphorylated saccharide is preferably about 1,000,000 or less, more preferably about 100,000 or less, furthermore preferably about 10,000 or less, for example, about 9,000 or less, about 8,000 or less, about 7,000 or less, about 6,000 or less, about 5,000 or less, about 4,000 or less, about 3,000 or less, and particularly preferably 2,000 or less, and in one embodiment, 1,000 or less.

The phosphorylated saccharide is in the form of an acid (i.e., hydrogen is bonded to a phosphate group). In the present invention, an ionized form of the phosphorylated saccharide (i.e., hydrogen of a phosphate group is dissociated and parted to leave phosphate ions) may be used, or a form of a salt (i.e., a phosphate ion and a positive ion of a base are bonded) may be used. In a specific embodiment, an inorganic salt of the phosphorylated saccharide is preferably used. The inorganic salt of the phosphorylated saccharide is preferably a calcium salt, a magnesium salt, a potassium salt, a zinc salt, an iron salt or a sodium salt. The calcium salt form of the phosphorylated saccharide is also referred to as phosphorylated saccharide calcium. The magnesium salt of the phosphorylated saccharide is also referred to as phosphorylated saccharide magnesium. The potassium salt of the phosphorylated saccharide is also referred to as phosphorylated saccharide potassium. The zinc salt of the phosphorylated saccharide is also referred to as phosphorylated saccharide zinc. The iron salt of the phosphorylated saccharide is also referred to as phosphorylated saccharide iron. The sodium salt form of the phosphorylated saccharide is also referred to as phosphorylated saccharide sodium. The same shall apply to other inorganic salts. The phosphorylated saccharides and salts thereof used in the present invention are preferably phosphorylated saccharides and salts thereof described in Japanese Laid-open Patent Publication No. 8-104696.

The saccharide moiety of the phosphorylated saccharide can be any saccharide. The saccharide moiety is preferably selected from the group consisting of a glucan, a reduced glucan, mannan, dextran, agar, cyclodextrin, fucoidan, gellan gum, locust bean gum, guar gum, tamarind gum and xanthan gum. A glucan or a reduced glucan is preferable. Herein, the reduced glucan refers to those in which aldehyde at a reducing terminal of a glucan is reduced into an alcohol. The reduced glucan is, for example, obtained by hydrogenising a glucan to reduce aldehyde into an alcohol.

The degree of polymerization of the glucan or reduced glucan, i.e. the number of glucose residues is preferably 2 or more, and more preferably 3 or more. The number of glucose residues is preferably about 100 or less, more preferably about 90 or less, more preferably about 80 or less, more preferably about 70 or less, more preferably about 60 or less, more preferably about 50 or less, more preferably about 40 or less, more preferably about 30 or less, more preferably about 20 or less, more preferably about 10 or less, more preferably about 9 or less, more preferably about 8 or less, furthermore preferably about 7 or less, more preferably about 6 or less, and particularly preferably about 5 or less.

There is no particular limitation on the number of inorganic ions in the phosphorylated saccharide inorganic salt. Inorganic ions may be bonded to all phosphate group(s) existing in the phosphorylated saccharide, or inorganic ions may be bonded to only a portion of them. In one molecule of the phosphorylated saccharide inorganic salt, only one inorganic ion may exist, two inorganic ions may exist, or three or more inorganic ions may exist. The number of inorganic ions in one molecule of the phosphorylated saccharide inorganic salt is preferably about 20 or less, more preferably about 10 or less, and still more preferably about 5 or less.

There is no particular limitation on the number of calcium ions in phosphorylated saccharide calcium. Calcium ions may be bonded to all phosphate group(s) existing in the phosphorylated saccharide, or calcium ions may be bonded to a portion thereof. In one molecule of phosphorylated saccharide calcium, only one calcium ion may exist, two calcium ions may exist, or three or more calcium ions may exist. The number of calcium ions in one molecule of phosphorylated saccharide calcium is preferably about 20 or less, more preferably about 10 or less, and still more preferably about 5 or less.

It is known that phosphorylated saccharide calcium has a remineralization effect on a tooth, a calcium absorption accelerating effect, and a taste quality improving effect.

In a preferred embodiment, a phosphorylated saccharide or an inorganic salt thereof, in which the saccharide moiety is a glucan or a reduced glucan and wherein at least one phosphate group(s) is bonded to this glucan or reduced glucan, is used. In still another preferred embodiment, a phosphorylated saccharide inorganic salt, in which the saccharide moiety is a glucan or a reduced glucan and wherein one to two phosphate group(s) are bonded to this glucan or reduced glucan and inorganic ions are bonded to each of these phosphate groups, is used.

In a more preferred embodiment, phosphorylated saccharide calcium, in which the saccharide moiety is a glucan or a reduced glucan and wherein at least one phosphate group(s) is bonded to this glucan or reduced glucan and calcium is bonded to at least one of these phosphate groups, is used. In still another preferred embodiment, phosphorylated saccharide calcium, in which the saccharide moiety is a glucan or a reduced glucan and wherein one to two phosphate groups are bonded to this glucan or reduced glucan and calcium is bonded to each of these phosphate groups, is used.

In still another preferred embodiment, a phosphorylated saccharide inorganic salt, in which the saccharide moiety is a glucan or a reduced glucan and wherein this glucan or reduced glucan is composed of α-1,4-bonded 3 to 5 glucose, and one phosphate group is bonded to this glucan or reduced glucan and inorganic ions are bonded to this phosphate group, is used.

In still another preferred embodiment, a phosphorylated saccharide calcium, in which the saccharide moiety is a glucan or a reduced glucan and wherein this glucan or reduced glucan is composed of α-1,4-bonded 3 to 5 glucose, and one phosphate group is bonded to this glucan or reduced glucan and calcium are bonded to this phosphate group, is used.

In still another preferred embodiment, an inorganic salt of a phosphorylated saccharide, in which the saccharide moiety is a glucan or a reduced glucan and wherein this glucan or reduced glucan is composed of α-1,4-bonded 2 to 8 glucose, and 1 to 2 phosphate groups are bonded to this glucan or reduced glucan and inorganic ions are bonded to at least one of the phosphate groups, and preferably to all the phosphate groups, is used.

In still another preferred embodiment, a phosphorylated saccharide calcium, in which the saccharide moiety is a glucan or a reduced glucan and wherein this glucan or reduced glucan is composed of α-1,4-bonded 2 to 8 glucose, and 1 to 2 phosphate groups are bonded to this glucan or reduced glucan and calcium are bonded to at least one of the phosphate groups, and preferably to all the phosphate groups, is used.

In still another preferred embodiment, a phosphorylated saccharide, in which the saccharide moiety is a glucan or a reduced glucan and wherein this glucan or reduced glucan contains α-1,4 bonded-glucose as a main chain and α-1,6 bonded- or α-1,4 bonded-glucose as a side chain, is used.

The phosphorylated saccharide and a salt thereof used in the present invention may be used as a pure single compound, or may be used as a mixture of plural kinds. The phosphorylated saccharide and a salt thereof used in the present invention is preferably a phosphorylated saccharide and a salt thereof described in Japanese Laid-open Patent Publication No. 8-104696. When a phosphorylated saccharide or a salt thereof is produced in accordance with the method described in Japanese Laid-open Patent Publication No. 8-104696, a mixture of plural kinds of phosphorylated saccharides or a salt thereof is obtained. The mixture may be used as it is, or only one kind of a compound may be selected and used after separating the mixture into pure compounds. The phosphorylated saccharide and a salt thereof exhibit excellent performance when used alone or as a mixture.

The phosphorylated saccharide can be produced, for example, by phosphorylating known saccharides. The phosphorylated saccharide inorganic salt can be produced, for example, by phosphorylating known saccharides to obtain a phosphorylated saccharide in the form of an acid and then converting the phosphorylated saccharide in the form of an acid into an inorganic salt. The phosphorylated saccharide calcium can be produced, for example, by phosphorylating known saccharides to obtain a phosphorylated saccharide in the form of an acid, and then converting the phosphorylated saccharide in the form of an acid into a calcium salt. The method of producing a phosphorylated saccharide and a salt thereof is described in Japanese Laid-open Patent Publication No. 8-104696. The phosphorylated saccharide calcium is also commercially available from EZAKI GLICO CO., LTD. as phosphorylated oligosaccharide calcium.

The saccharides which are the raw material for the production of the phosphorylated saccharide and a salt thereof include a glucan, mannan, dextran, agar, cyclodextrin, fucoidan, gellan gum, locust bean gum, guar gum, tamarind gum and xanthan gum. The case of a glucan will be described below. A common crude plant starch, preferably a starch in which a lot of phosphate groups are bonded, such as a crude starch from potato is suitable, but a purified product may also be used. A chemically modified starch can also be suitably used. Furthermore, it is also possible to use various carbohydrates in which phosphate group(s) are chemically bonded. In the potato starch, comparatively lots of phosphate groups are ester-bonded at the 3- and 6-positions of the glucose constituting the same. The phosphate group mainly exists in amylopectin.

In a preferred embodiment, in the case where the saccharide is a glucan, the phosphorylated saccharide can be obtained by degrading a starch or chemically modified starch having phosphate group(s).

In a preferred embodiment, an amylolytic enzyme, a glycosyltransferase, or α-glucosidase, or a combination of one or more kinds of them (excluding only one kind of α-glucosidase) is reacted with a starch having phosphate group(s) or a chemically modified starch.

In a preferred embodiment, the amylolytic enzyme is composed of a combination of one or more kinds of α-amylase, β-amylase, glucoamylase, isoamylase, pullulanase and neopullulanase. In a preferred embodiment, the glycosyltransferase described above is a cyclodextrin glucanotransferase.

In a preferred embodiment, according to the production method described above, a glycosyltransferase is allowed to act on a saccharide having phosphate group(s). The glycosyltransferase described above is a cyclodextrin glucanotransferase.

The phosphorylated saccharide inorganic salt is produced, for example, by reacting a salt of an alkali earth metal or a salt of iron with a phosphorylated saccharide in the form of an acid. The phosphorylated saccharide calcium is produced, for example, by reacting a calcium salt with a phosphorylated saccharide in the form of an acid.

As the phosphorylated saccharide and a salt thereof, those with high purities may be used, or those with low purities may be used. For example, the phosphorylated saccharide and a salt thereof may be used as a mixture with other saccharides. It is noted that in the present description, in the case of mentioning the concentration and the content of the phosphorylated saccharide and a salt thereof, the concentration and the content are calculated based on the amount of the pure phosphorylated saccharide and a salt thereof. Therefore, in the case where a mixture containing those other than the phosphorylated saccharide and a salt thereof is used, the concentration and the content are not calculated based on the amount of the entire mixture, but on the amount of the phosphorylated saccharide and a salt thereof in the mixture.

(2b. Water-Soluble Calcium Salt)

In a specific embodiment of the present invention, a water-soluble calcium salt is used. In the present description, the “water-soluble calcium salt” refers to a calcium salt whose solubility in water at 20° C. is 1% by weight or more. The solubility in water at 20° C. of the water-soluble calcium salt used in the present invention is preferably about 2% by weight or more, more preferably about 3% by weight or more, still more preferably about 4% by weight or more, and particularly preferably about 5% by weight or more. The definition of the water-soluble calcium salt also includes a phosphorylated saccharide calcium salt. Other examples of the water-soluble calcium salt include calcium chloride, organic acid calcium salts (for example, calcium lactate, calcium gluconate, calcium acetate, calcium glutamate, calcium lactobionate, fermented calcium, calcium citrate, calcium citrate malate, calcium formate, calcium benzoate, calcium isolactate, calcium propionate, calcium salicylate, and calcium ascorbate), colloidal calcium carbonate, calcium polyol phosphate, calcium hydroxide, calcium carbonate, calcium hydrogen phosphate, calcium phosphate, whey calcium, casein phosphopeptide calcium, and calcium fluoride.

(2c. Fluoride)

In the present invention, fluoride is used. It is known that, although fluoride ions react easily with calcium ions to cause precipitation, a state of calcium ions and fluoride ions is maintained by the presence of a phosphorylated saccharide (Patent Document 1 (Japanese Laid-open Patent Publication No. 2002-325557)). Therefore, recrystallization of the demineralized affected part can be accelerated by supplying fluoride together with calcium ions and phosphate ions. Furthermore, acquisition of acid resistance can be expected by incorporating fluoride ions into a crystal. In the present invention, it is preferred to design the present invention so that fluoride is released simultaneously with or after the release of a water-soluble calcium salt. In the present invention, it is preferred to design the present invention so that fluoride is released simultaneously with or after the release of a phosphorylated saccharide or a salt thereof.

Conventionally, fluoride is frequently used in a high concentration of 1,000 ppm or more. In the present invention, it is possible to ensure a sufficient amount of fluoride ions by using a phosphorylated saccharide or a salt thereof and a polyphenol simultaneously with a fluoride even when a fluoride is used in a lower concentration as compared with a conventional case. Therefore, use of a low concentration of fluoride makes it possible to obtain the effect equal to or higher than that of a conventional high-concentration fluoride. According to the present invention, a sufficient effect can be obtained even by the addition of fluoride of, for example, 100 ppm or less, and preferably use of fluoride of 10 ppm or less.

The fluoride is preferably a compound which dissolves in water to release fluoride ions. The fluoride is preferably fluoride in which the formulation is approved for food, pharmaceuticals or quasi-drugs. Examples of such fluoride include sodium fluoride, potassium fluoride, monofluorophosphoric acid and a salt thereof (for example, sodium monofluorophosphate), calcium fluoride, strontium fluoride, cryolite, and monofluoroacetic acid. In a specific embodiment, it is preferred to use, as the fluoride, potassium fluoride, sodium monofluorophosphate, strontium fluoride or tea-derived fluoride in the food or the composition of the present invention. In an invention of a food, it is preferred to use, as the fluoride, fluoride which can be used as a food (for example, fluoride derived from tea, well water, seawater, fishery products, sea grass or the like).

(2d. Polyphenol)

A polyphenol is generally a generic term for compounds having plural phenolic hydroxy groups (hydroxy groups bonded to an aromatic ring such as a benzene ring or a naphthalene ring) in the molecule.

Examples of a typical polyphenol include flavonoid, phenolic acid, chlorogenic acid, ellagic acid, lignan, curcumin, and coumarin.

Examples of flavonoids include catechin, anthocyanin, tannin, rutin, and isoflavone.

A large amount of catechin is contained in wine, apple, blueberry, tea, cacao and the like.

A large amount of anthocyanin is contained in the fruit skin of grape, purple fleshed sweet potato, and a reddish purple fruit such as blueberry.

Tannin is contained in tea, red wine, persimmon, banana and the like.

Rutin is a kind of vitamin P and is contained in buckwheat.

Isoflavone is contained in soybean, kudzu, kudzu starch and the like.

Examples of phenolic acid include chlorogenic acid. A large amount of chlorogenic acid is contained in coffee.

Ellagic acid is contained in strawberry and the like.

A large amount of lignan is contained in sesame.

A large amount of curcumin is contained in turmeric.

A large amount of coumarin is contained in cherry leaves, parsley, peach, citruses and the like.

A polyphenol may be derived from a natural product, or may be chemically synthesized. A polyphenol is preferably derived from a natural product, and more preferably derived from a plant.

In the present invention, any polyphenol can be used. Examples of the polyphenol that can be used in the present invention include tannin.

The polyphenol is produced in natural products as a mixture of polyphenols having various structures, and it is often difficult to obtain a pure compound. In the present invention, it is preferred to use, as the polyphenol, a mixture of polyphenols contained in a tea extract (referred to as a “tea polyphenol” in the present description).

(2e. Tea Extract)

In the present invention, it is preferred to use a tea extract. This is because the tea extract contains both fluoride and polyphenol. However, a conventional tea extract contains a large amount of polyphenol as compared to the amount of fluoride. Therefore, in the present invention, it is preferred to use a low polyphenol content-tea extract from which most of the polyphenol in a conventional tea extract has been removed. The polyphenol contained in a preferable low polyphenol-high fluoride content tea extract used in the present invention preferably contains a mixture of catechin, gallocatechin, catechin gallate, gallocatechin gallate, epicatechin, epigallocatechin, epicatechin gallate and epigallocatechin gallate, as the main component. The total weight of these compounds in the polyphenols contained in the low polyphenol-high fluoride content tea extract is preferably about 60% by weight or more, more preferably about 70% by weight or more, furthermore preferably about 80% by weight or more, particularly preferably about 90% by weight or more and most preferably about 95% by weight. The total weight of these compounds in the low polyphenol-high fluoride content tea extract is preferably about 60% by weight or more, more preferably about 70% by weight or more, furthermore preferably about 80% by weight or more, particularly preferably about 85% by weight or more and most preferably about 90% by weight or more, of the tea extract. The total weight of these compounds in the low polyphenol-high fluoride content tea extract is preferably about 99% by weight or less, more preferably about 98% by weight or more, further more preferably about 97% by weight or more, particularly preferably about 95% by weight or more and most preferably about 90% by weight or less, of the tea extract.

The tea extract suitable for use in the present invention contains a very high concentration of fluoride and a very low concentration of a tea polyphenol. Regarding the ratio of the concentration of fluoride to the concentration of the tea polyphenol in the tea extract used suitably in the present invention, the concentration of the tea polyphenol is preferably about 200 or less, more preferably about 100 or less, furthermore preferably about 25-50 or less, particularly preferably about 40 or less and most preferably about 30 or less, assuming that the concentration of fluoride is 1. A ratio of the concentration of fluoride to the concentration of the tea polyphenol in the tea extract used in the present invention can be, for example, about 1 or more, about 3 or more, about 5 or more, about 10 or more and about 15 or more, assuming that the concentration of fluoride is 1.

Herein, a ratio of the amount of fluoride to the amount of tea polyphenols in a conventional tea extract will be described below. The amount of fluoride in a dry product of a tea extract obtained by extracting tea using a conventional method is from about 100 ppm to about 400 ppm, and the amount of the polyphenols is from about 30% by weight to about 40% by weight. That is, the amount of the polyphenols is about 1,000 times to about 4,000 times the amount of fluoride.

The tea extract used in the present invention is produced, for example, in the following manner.

A tea raw material is used as an extraction raw material. The tea raw material refers to a processed product derived from plant bodies such as leaves, stems and buds of a tea plant (Camellia sinensis) belonging to the genus Camellia. Processing may be any action as long as it is an action of changing a state of a plant bodies by acting on the plant bodies, such as steaming, drying, freezing, grinding, and the like of plant bodies. The tea raw material is a product obtained as a result of such a process. The tea raw material may be obtained by a single process, or may be obtained by plural processes. Any tea product used for various applications such as drinking, flavoring, and seasoning can be used as the tea raw material in the present invention.

The tea raw material can be preferably a dry product from plant bodies of a tea plant. Such a dry product may be obtained by fermenting components contained in plant bodies of the tea plant, or may be obtained without fermentation. Such a dry product is classified into a non-fermented tea, a semi-fermented tea and a fermented tea depending on the degree of fermentation. The non-fermented tea is a tea which is not fermented in the production process. Examples of the non-fermented tea include green tea. Green tea is classified into sencha-green tea, bancha-coarse green tea, roasted green tea, gyokuro-superior green tea, powdered green tea, and the like depending on the difference in the production method. Semi-fermented tea is a tea which is fermented to a medium degree in the production process. Examples of semi-fermented tea include oolong tea. Fermented tea is a tea which is completely fermented in the production process. Examples of the fermented tea include black tea.

The tea raw material is preferably subjected to extraction in a ground state (for example, a fragment or a powder). Such a ground product (for example, a fragment or a powder) is easily available to those skilled in the art. Such a ground product is available, for example, by grinding or crushing dried tea leaves. Alternatively, such a powder can also be obtained by freeze-grinding fresh tea leaves.

The solvent used in extracting the tea raw material is any solvent known in the relevant art. Examples of solvent used in the extraction of the tea raw material include water (including warm water), and organic solvents (for example, ether, ethanol, a mixture of ethanol and water, and acetone). Such a solvent is preferably water (including warm water), and more preferably water at about 80° C. to about 100° C.

After extraction, excess polyphenols (particularly, tannin and catechin) are removed from this tea extraction liquid by column chromatography, by a method of purifying a hot water extract of tea using an activated carbon column, or the like.

As described above, the amount of the polyphenols in tea or tea extract is usually about 1,000 times to about 4,000 times of the amount of fluoride. Therefore, in the case of using a tea raw material in the present invention, it is preferred that the amount of the polyphenols is decreased without decreasing the amount of fluoride as much as possible, in other words, the amount of the polyphenols is relatively decreased as a ratio to the amount of fluoride. It is possible to use, as the method of decreasing the amount of the polyphenols without decreasing the amount of fluoride as much as possible, any known method of removing polyphenol from a tea raw material. For example, the amount of polyphenols can be decreased by allowing a tea raw material liquid to flow through a column using a material which does not adsorb a fluoride compound but adsorbs a polyphenol.

(2f. Phosphoric Acid Source Compound)

A Ca/P ratio of hydroxyapatite (which is represented by Ca₁₀(PO₄)₆(OH)₂) which is the main component of tooth enamel is about 1.67 and, in a composition constituting tooth enamel, the Ca/P ratio is from about 1.0 to about 1.67 (P/Ca ratio=0.6 to 1.0). Therefore, in order to enable the Ca/P ratio to come close to about 1.0 to about 1.67 (P/Ca ratio=0.6 to 1.0), and preferably about 1.67 (P/Ca ratio=0.6), remineralization of the enamel can be accelerated by supplying phosphate ions and calcium ions.

In the present invention, when only a phosphorylated saccharide calcium salt is used, or only a combination of a phosphorylated saccharide salt other than a calcium salt or a phosphorylated saccharide and a water-soluble calcium salt is used, only calcium ions are supplied and there is a lack of phosphate ions for remineralization with higher efficiency as it is.

It is known that a large amount of phosphoric acid exists in saliva. In a normal human body, a molar ratio calcium:phosphoric acid (hereinafter referred to as a “Ca/P ratio”) in saliva is generally from about 0.25 to about 0.67 (P/Ca=about 1.45 to about 3.9), and phosphoric acid excessively exists (i.e., approximately phosphoric acid (3 mol):calcium (2 mol) to phosphorus (3.9 mol):calcium (1 mol)). Therefore, in the case of a food which is well chewed in the oral cavity, like a chewing gum, lack of phosphoric acid hardly arises without adding phosphoric acid.

However, in the case of a food such as juice which washes away saliva, and a composition which is directly applied to a tooth surface, there may be a lack of phosphate ions for remineralization with higher efficiency. Therefore, in the composition and the food of the present invention, it is also preferred to simultaneously use a supply source of phosphate ions. In the present description, the supply source of phosphate ions is referred to as a phosphoric acid source compound. The phosphoric acid source compound means a phosphoric acid compound.

The phosphoric acid source compound which can be used in the present invention can be any compound as long as it dissolves in water to release phosphate ions. The phosphoric acid source compound is preferably a water-soluble phosphate or inorganic phosphoric acid. Examples of such a phosphoric acid source compound include phosphoric acid, sodium phosphate, potassium phosphate, polyphosphoric acid and salts thereof, and cyclic phosphoric acid and salts thereof. Examples of the sodium phosphate include sodium metaphosphate, disodium hydrogen phosphate, sodium dihydrogen phosphate, trisodium phosphate, sodium pyrophosphate, and sodium hydrogen pyrophosphate. Examples of the potassium phosphate include potassium dihydrogen phosphate, dipotassium hydrogen phosphate, and tripotassium phosphate. The polyphosphoric acid is a compound formed by condensation of two or more phosphoric acids. The degree of polymerization in polyphosphoric acid is any degree as long as it is 2 or more and is, for example, 2 or more and 10 or less. Examples of the polyphosphoric acid include pyrophosphoric acid, triphosphoric acid, trimetaphosphoric acid, tetrametaphosphoric acid, and cyclopolyphosphoric acid. A salt of these polyphosphoric acids can also be used and is preferably a sodium salt, a potassium salt or a magnesium salt. Examples of the cyclic phosphoric acid include hexametaphosphoric acid. A salt of these cyclic phosphoric acids can also be used and is preferably a sodium salt, a potassium salt or a magnesium salt.

This phosphoric acid source compound can be added alone or in combination to the composition and the food of the present invention so as to allow the Ca/P ratio to come closer to about 1.0 to about 2.0 (P/Ca ratio=about 0.5 to about 1.0), and preferably about 1.67 (P/Ca ratio=about 0.6).

(2f. Other Materials)

In the composition and the food of the present invention, as long as the remineralization action and the tooth surface strengthening action are not disturbed, it is possible to use any material which is usually used in the objective composition and food.

In the case where the food of the present invention is, for example, a chewing gum, it is possible to contain a gum base, a sweetener, a gelatine, a flavor, a brightener, a coloring agent, a thickener, an acidulant, a pH adjustor and the like. Examples of the gum base include chicle, vinyl acetate, ester gum, polyisobutylene and a styrene-butadiene rubber. The sweetener can be a saccharide, a sugar alcohol, a high-sweetness sweetener or the like. The sweetener is preferably non-cariogenic so as to prevent dental caries. The sweetener is more preferably selected from maltitol, reduced palatinose, palatinose, lactitol, erythritol, sorbitol, xylitol, an aspartame L-phenylalanine compound, trehalose and mannitol. The formulation of a chewing gum can be in accordance with the formulation known in the relevant art.

In the case where the food of the present invention is, for example, a candy, the food can contain saccharides such as sucrose and starch syrup, wheat flour, condensed milk, common salt, agar, gelatine, nuts (peanut, etc.), shortening, butter, an acidulant, a flavor, a pH adjustor, a coloring agent and the like. The saccharides can be a saccharide, a sugar alcohol, a high-sweetness sweetener or the like. The saccharides are preferably non-cariogenic saccharides so as to prevent dental caries. The saccharides are more preferably selected from maltitol, reduced palatinose, palatinose, lactitol, erythritol, sorbitol, xylitol, an aspartame L-phenylalanine compound, trehalose and mannitol. The formulation of a candy can be in accordance with the formulation known in the relevant art.

A tablet candy (also referred to as a tablet) refers to a food which is formed by compression molding of a powder or a granule and is designed so that it is gradually dissolved or disintegrated in the mouth and acts persistently in the oral cavity for a long time. The time required for the tablet candy from initiation of dissolving in the oral cavity to the completion of dissolving depends on the size and the raw material of the tablet candy. Those skilled in the art can arbitrarily design and produce a tablet candy suitable for achieving a desired time for initiation of dissolving to the completion of dissolving of the tablet candy. Examples of the raw material used in the tablet candy include the following: saccharides, calcium carbonate, calcium phosphate, calcium sulfate, powder cellulose, an emulsifier, an acidulant, a flavor, a pH adjustor and a coloring agent. The saccharides are preferably non-cariogenic saccharides so as to prevent dental caries. The saccharides can be saccharides (sucrose, starch syrup, lactose, glucose, starch and the like), sugar alcohols, high-sweetness sweeteners, or the like. The saccharides are more preferably selected from maltitol, reduced palatinose, palatinose, lactitol, erythritol, sorbitol, xylitol, an aspartame L-phenylalanine compound, trehalose and mannitol. The formulation of a tablet candy can be in accordance with the formulation known in the relevant art.

On the other hand, dental caries is a disease caused by bacteria. Therefore, in the composition and the food of the present invention, use in combination with an antibacterial agent or a dental plaque formation inhibitor is also effective. It is also known that hydroxyapatite adsorbs cariogenic bacteria. Examples of a sterilizer and an antibacterial agent include benzalkonium chloride, cetylpyridinium chloride, paraben, benzoic acid, and alcohols such as ethanol. The substances having comparatively high safety include combinations with chitin-chitosan, chitosan oligosaccharide, lactoferrin, polyphenol, and the like. It is also possible to use the composition and the food in combination with a drug which inhibits inflammation caused by bacteria. Typical anti-inflammatory agents include flavonoids such as genistein and naringenin, polyamine, β-glucan, alkaloid, hesperidin, hesperetin, and glucosyl hesperidin. These various drugs can be contained in the composition and the food of the present invention, if necessary.

### 3. Food of the Present Invention

In one embodiment, the food of the present invention is a cariostatic food comprising:

(1) (i) a phosphorylated saccharide calcium salt; or (ii) a combination of a phosphorylated saccharide salt other than a phosphorylated saccharide calcium salt or a phosphorylatedsaccharide, and a water-soluble calcium salt other than a phosphorylated saccharide calcium salt;

(2) a fluoride; and

(3) a polyphenol; wherein

the phosphorylated saccharide is composed of a saccharide moiety and phosphate group(s);

the content of the component (1) in the food is in an amount suitable to make the calcium concentration in saliva in the oral cavity to be 1 mM to 12 mM when the food exists in the oral cavity;

the content of the fluoride in the food is in an amount suitable to make the fluoride concentration in saliva in the oral cavity to be 0.2 ppm to 100 ppm when the food exists in the oral cavity;

the content of the polyphenol in the food is in an amount suitable to make the polyphenol concentration in saliva in the oral cavity to be 0.001% by weight to 0.1% by weight when the food exists in the oral cavity; and

the food remains in the oral cavity for 5 minutes or more upon eating.

In one embodiment, the food of the present invention is a cariostatic food comprising:

(1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt;

(2) a fluoride; and

(3) a polyphenol; wherein

the phosphorylated saccharide is composed of a saccharide moiety and phosphate group(s);

the content of the component (1) in the food is in an amount suitable to make the calcium concentration in saliva in the oral cavity to be 1 mM to 12 mM when the food exists in the oral cavity;

the content of the fluoride in the food is in an amount suitable to make the fluoride concentration in saliva in the oral cavity to be 0.2 ppm to 100 ppm when the food exists in the oral cavity;

the content of the polyphenol in the food is in an amount suitable to make the polyphenol concentration in saliva in the oral cavity to be 10 times to 200 times the fluoride concentration when the food exists in the oral cavity; and

the food remains in the oral cavity for 5 minutes or more upon eating.

In one embodiment, the food of the present invention is a cariostatic food comprising:

(1) (i) a phosphorylated saccharide calcium salt; or (ii) a combination of a phosphorylated saccharide salt other than a phosphorylated saccharide calcium salt or a phosphorylatedsaccharide, and a water-soluble calcium salt other than a phosphorylated saccharide calcium salt;

(2) a fluoride; and

(3) a polyphenol; wherein

the phosphorylated saccharide is composed of a saccharide moiety and phosphate group(s);

the content of the component (1) in the food is in an amount suitable to make the calcium concentration in saliva in the oral cavity to be 1 mM to 12 mM when the food exists in the oral cavity;

the content of the fluoride in the food is in an amount suitable to make the fluoride concentration in saliva in the oral cavity to be 0.005 times to 0.1 times the concentration of calcium derived from the component (1) when the food exists in the oral cavity;

the content of the polyphenol in the food is in an amount suitable to make the polyphenol concentration in saliva in the oral cavity to be 10 times to 200 times the fluoride concentration when the food exists in the oral cavity; and

the food remains in the oral cavity for 5 minutes or more upon eating.

(3a. Method for Producing Food of the Present Invention)

The food of the present invention can be produced so as to contain (1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt; (2) a fluoride; and (3) a polyphenol by any method known in the relevant art.

In the case of above described (ii), it is preferred that the food of the present invention contain a combination of a phosphorylated saccharide salt other than a phosphorylated saccharide calcium salt or a phosphorylated saccharide, and a water-soluble calcium salt other than a phosphorylated saccharide calcium salt, substantially homogeneously. A food homogeneously containing these materials has an advantage that it can be produced easily.

In the case of the above described (ii), the component may be separated into a portion containing a phosphorylated saccharide salt or a phosphorylated saccharide, and a portion containing a water-soluble calcium salt other than a phosphorylated saccharide calcium salt. In this case, the food of the present invention should be designed so that water-soluble calcium salt other than phosphorylated saccharide calcium salt is released from the food, simultaneously with or after release of phosphorylated saccharide salt other than phosphorylated saccharide calcium salt or phosphorylated saccharide. The reason is that when the water-soluble calcium salt other than phosphorylated saccharide calcium salt is released prior to the phosphorylated saccharide or a salt thereof, calcium ions disorderly deposit on the tooth surface, unfavorably.

In the food of the present invention, fluoride is also used. The fluoride should be designed so as to be released simultaneously with the phosphorylated saccharide salt or phosphorylated saccharide, or after the phosphorylated saccharide salt or phosphorylated saccharide.

In the food of the present invention, a polyphenol is also used. The polyphenol should be designed so as to be released simultaneously with the phosphorylated saccharide salt or phosphorylated saccharide.

In the food of the present invention, a phosphoric acid source compound can also be used. In that case, it is preferred to design the food so that the phosphoric acid source compound is released simultaneously with the phosphorylatedsaccharide salt or phosphorylated saccharide or after the phosphorylated saccharide salt or phosphorylated saccharide.

These facts are applied to all food and compositions of the present invention.

(3b. Food of the Present Invention)

The food of the present invention can be any food which contains (1) (i) a phosphorylated saccharide calcium salt; or (ii) a combination of a phosphorylated saccharide salt other than a phosphorylated saccharide calcium salt or a phosphorylatedsaccharide, and a water-soluble calcium salt other than a phosphorylated saccharide calcium salt; (2) a fluoride; and (3) a polyphenol. In the case where the food of the present invention contains aphosphorylated saccharide calcium salt, the food of the present invention does not have to further contain a phosphorylated saccharide or a salt thereof, but it may contain the phosphorylated saccharide or a salt thereof.

Examples of the food of the present invention include chewing gums; candies; tablet candies; complex beverages; semi-fluid food such as yogurt; baked confectioneries such as biscuit and rice cracker; frozen desserts such as ice cream; gelled food such as jelly; and noodles. Chewing gums, candies and tablet candies enable an active ingredient to remain in the oral cavity for a long time and are therefore suitable as the food of the present invention. It is known that stimulated saliva already contains calcium ions in the concentration of about 1 to 1.5 mM, and thus it is desirable to pay attention to it upon product design.

In the case where the food of the present invention is chewing gum, the chewing gum can be a sugar-coated gum or a stick gum. It is preferred that all portions of the chewing gum contain (1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt; (2) a fluoride; and (3) a polyphenol. In the case where the gum is a sugar-coated gum and contains a combination of a phosphorylated saccharide salt other than a phosphorylated saccharide calcium salt or a phosphorylated saccharide, and a water-soluble calcium salt other than a phosphorylated saccharide calcium salt, it is preferred that the sugar coating portion contains a phosphorylated saccharide salt other than a phosphorylated saccharide calcium salt or a phosphorylated saccharide, and the gum portion contains a water-soluble calcium salt other than a phosphorylated saccharide calcium salt. In the case where the chewing gum is a stick gum and contains a combination of a phosphorylated saccharide salt other than a phosphorylated saccharide calcium salt or a phosphorylated saccharide, and a water-soluble calcium salt other than a phosphorylated saccharide calcium salt, it is preferred that the chewing gum is a stick gum containing a microcapsule, the gum portion contains a phosphorylated saccharide salt other than a phosphorylated saccharide calcium salt or a phosphorylated saccharide, and the microcapsule contains a water-soluble calcium salt other than a phosphorylated saccharide calcium salt. In both cases, the phosphorylated saccharide may be contained in either a phosphorylated saccharide-containing portion or a calcium-containing portion, or both of them.

In the case where the food of the present invention is a candy, the candy may be either a single-layered candy or a plural-layered candy. The candy refers to a food which contains saccharides such as sucrose and starch syrup as main materials, and is produced by a method including the step of boiling down saccharides. Candies are classified into a soft candy and a hard candy. Examples of the soft candies include soft caramel, hard caramel, nougat and marshmallow. Examples of the hard candies include drop, taffy and brittle.

In the case where the food of the present invention is a single-layered candy, it is preferred that this candy contains (1) (i) a phosphorylated saccharide calcium salt; or (ii) a combination of a phosphorylated saccharide salt other than a phosphorylated saccharide calcium salt or a phosphorylatedsaccharide, and a water-soluble calcium salt other than a phosphorylated saccharide calcium salt; (2) a fluoride; and (3) a polyphenol, substantially homogeneously. The single-layered candy has such an advantage that it is easy to produce as compared with the plural-layered candy.

In the case where the plural-layered candy is a candy composed of two layers, i.e. a center layer and a coating layer which surrounds the center layer, both the center layer and the coating layer may contain, for example, (1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt; (2) a fluoride; and (3) a polyphenol, or either one of the center layer and the coating layer may contain them. In one embodiment, it is preferred that the center layer contains a phosphorylated saccharide salt other than a phosphorylated saccharide calcium salt or a phosphorylated saccharide, and the coating layer contains a water-soluble calcium salt other than a phosphorylated saccharide calcium salt. The center layer may be a hard candy or a soft candy, or a cream. The coating layer may be a hard candy, a soft candy, a sugar coating, or a layer of a powder. The candy of the present invention is not limited to a one-layered candy and a two-layered candy, and may be provided with further layers.

In one embodiment, the food of the present invention may be a confectionery including a gum coated with a candy (also referred to as a sugar-coated candy gum). In this case, for example, both the candy and the gum may contain (1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt; (2) a fluoride; and (3) a polyphenol, or only either one of the candy and the gummay contain them. In the case where the food of the present invention is a sugar-coated candy gum and contains (ii) a combination of a phosphorylated saccharide salt other than a phosphorylated saccharide calcium salt or a phosphorylated saccharide, and a water-soluble calcium salt other than a phosphorylated saccharide calcium salt, the sugar-coated candy gum may have such a constitution that the candy portion contains a phosphorylated saccharide salt other than a phosphorylated saccharide calcium salt or a phosphorylated saccharide, and the candy portion contains a water-soluble calcium salt other than a phosphorylated saccharide calcium salt.

In the case where the food of the present invention is a tablet candy, the tablet candy may either be a single-layered tablet candy or a plural-layered tablet candy. In the case where the food of the present invention is a single-layered tablet candy, it is preferred that this tablet candy contains (1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt; (2) a fluoride; and (3) a polyphenol, substantially homogeneously. The single-layered tablet candy has such an advantage that it is easy to produce as compared to the plural-layered tablet candy.

In the case where the food of the present invention is a plural-layered tablet candy, for example, all layers may contain (1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt; (2) a fluoride; and (3) a polyphenol, or only any one or two layers may contain them. In the case where the food of the present invention is a three-layered tablet candy composed of three layers and contains (ii) a combination of a phosphorylated saccharide salt other than a phosphorylated saccharide calcium salt or a phosphorylated saccharide, and a water-soluble calcium salt other than a phosphorylated saccharide calcium salt, it is preferred that a middle layer sandwiched between the two layers contains a water-soluble calcium salt other than a phosphorylated saccharide calcium salt, and the two layers sandwiching this layer contain a phosphorylated saccharide salt other than a phosphorylated saccharide calcium salt or a phosphorylated saccharide.

In the case where the food of the present invention is a frozen dessert such as ice cream, it is preferred that the food of the present invention contains (1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt; (2) a fluoride; and (3) a polyphenol, substantially homogeneously.

In another embodiment, the food of the present invention can be a frozen dessert containing a solid food in a frozen dessert which is a base. In this case, for example, both of the frozen desserts which is a base and a solid food may contain (1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt; (2) a fluoride; and (3) a polyphenol, or either of the frozen dessert which is a base or a solid food may contain them. In the case where the food of the present invention is a frozen dessert containing a solid food in a frozen dessert which is a base, and contains (ii) a combination of a phosphorylated saccharide salt other than a phosphorylated saccharide calcium salt or a phosphorylated saccharide, and a water-soluble calcium salt other than a phosphorylated saccharide calcium salt, the frozen dessert may have such a constitution that the frozen dessert which is a base contains a phosphorylated saccharide salt other than a phosphorylated saccharide calcium salt or a phosphorylated saccharide, and the solid food contains a water-soluble calcium salt other than a phosphorylated saccharide calcium salt.

Examples of frozen desserts of the present invention or such a frozen dessert which is a base include ice cream, ice milk, lact ice and sherbet. Such a solid food can be, for example, a gel. Examples of such a solid food include tapioca, nata de coco, agar, jelly, bavarois, and jam. Such a solid food can have any size and preferably has a diameter of 2 mm or more, and more preferably has a diameter of 3 mm or more. The diameter of a solid food may be, for example, 4 mm or more, 5 mm or more, 6 mm or more, 7 mm or more, 8 mm or more, 9 mm or more, or 10 mm or more. The diameter of a solid food is preferably 15 mm or less, more preferably 14 mm or less, and still more preferably 13 mm or less. The diameter of a solid food may be, for example, 12 mm or less, 11 mm or less, 10 mm or less, 9 mm or less, 8 mm or less, 7 mm or less, 6 mm or less, or 5 mm or less.

The weight of the food of the present invention can be any weight. The weight of the food of the present invention is preferably about 0.05 g or more, more preferably about 0.1 g or more, and still more preferably about 0.5 g or more. The weight of the food of the present invention is preferably about 5 g or less, more preferably about 4 g or less, and still more preferably about 3 g or less.

In the case where the food of the present invention is a chewing gum, the weight of the chewing gum is preferably about 0.05 g or more, more preferably about 0.1 g or more, and still more preferably about 0.5 g or more. The weight of the chewing gum is preferably about 3 g or less, more preferably about 2 g or less, and still more preferably about 1 g or less.

In the case where the food of the present invention is a candy, the weight of the candy is preferably about 0.5 g or more, more preferably about 1 g or more, and still more preferably about 1.5 g or more. The weight of the candy is preferably about 5 g or less, more preferably about 4 g or less, and still more preferably about 3 g or less.

In the case where the food of the present invention is a tablet candy, the weight of the tablet candy is preferably about 0.05 g to about 10 g, more preferably about 0.1 g to about 5 g, and still more preferably about 0.2 g to about 3 g.

The food of the present invention can have any shape. For example, when the food of the present invention is a chewing gum, a candy or a tablet candy, it can have a disk shape, a spherical shape, a rugby football shape, a heart shape or the like. For example, when the food of the present invention is a complex beverage, yogurt or the like, it does not have any particular shape.

In one embodiment, in the case where the food of the present invention contains a phosphorylated saccharide or a salt thereof (excluding a calcium salt), the content of the phosphorylated saccharide and a salt thereof in the food of the present invention can be arbitrarily set taking the form of the food, the dilution ratio upon eating and the like into consideration. For example, the content (total) of the phosphorylated saccharide and a salt thereof in the food of the present invention is in an amount suitable to make the concentration of a phosphorylated saccharide in saliva in the oral cavity to be preferably about 1.0 mM or more, more preferably about 1.5 mM or more, furthermore preferably about 2.0 mM or more, particularly preferably about 2.5 mM or more, and most preferably about 3.0 mM or more, when the food exists in the oral cavity. For example, the content (total) of the phosphorylated saccharide and a salt thereof in the food of the present invention is in an amount suitable to make the concentration of a phosphorylated saccharide in saliva in the oral cavity to be preferably about 12 mM or less, more preferably about 6 mM or less, furthermore preferably about 5 mM or less, particularly preferably about 4.5 mM or less, and most preferably about 4 mM or less, when the food exists in the oral cavity.

When used in the present description in the context of a food, “the content is in an amount suitable to make the concentration of it in saliva in the oral cavity to be 1.0 mM or more when the food exists in the oral cavity” refers to an amount suitable to make the concentration, which is obtained by collecting a liquid produced in the oral cavity during 20 minutes from starting to eat food of the present invention and measuring the concentration of the component in the liquid, to be 1.0 mM. For example, a method of collecting the liquid 20 times every minute can be employed. In that case, a measuring sample can be obtained by combining the liquid collected 20 times. It is preferred that the food is kept in the oral cavity during these 20 minutes without swallowed. Alternatively, the food may be put in the mouth little by little during the 20 minutes, followed by chewing. Then, it is possible to employ such a method in which every time when the eater feels the accumulation of saliva in the oral cavity, the eater is allowed to expel saliva and the expelled liquid is collected. However, the eater is refrained from expelling the food when expelling saliva. The same interpretation is possible for other concentrations. In the present description, the term “saliva” is not pure saliva secreted from the salivary gland, but refers to a liquid accumulated in the oral cavity in the case of chewing food in the oral cavity. In this case, the liquid accumulated in the oral cavity is a mixture of pure saliva, a liquid portion derived from the food, and various solutes derived from the food. The amount of each component to be added in the food varies according to the weight, size and the like of the food. In the case where an intake per time of the food is large, each component is added so that the content becomes lower than that in the case of a small intake. For example, in order to achieve the same use amount, the added amount (%) in 2 g of the food is about 0.5 times the added amount (%) in 1 g of the food. Human saliva is secreted in an amount of about 20 mL on average during the 20 minutes. Therefore, the amount added in the food is set considering the amount to be eluted relative to 20 mL of saliva. The setting of such an amount to be added can be easily done by those skilled in the art.

In the case where the food is a chewing gum containing a phosphorylated saccharide or a salt thereof, when this gum is chewed in the oral cavity for about 20 minutes, almost all of the phosphorylated saccharide and salt thereof contained in this gum will be eluted in saliva within 20 minutes.

In the case where the food is a chewing gum containing fluoride together with a phosphorylated saccharide or a salt thereof, when this gum is chewed in the oral cavity for about 20 minutes, the fluoride eluted in saliva within 20 minutes will be about 50% to about 60% of fluoride contained in this gum.

In the case where the food is a chewing gum containing a phosphoric acid source compound, when this gum is chewed in the oral cavity for about 20 minutes, almost all of the phosphoric acid source compound contained in this gum will be eluted in saliva within 20 minutes.

In one embodiment, the content of a water-soluble calcium salt (including phosphorylated saccharide calcium) in the food of the present invention can be arbitrarily set taking the form of the food, the dilution ratio upon eating and the like into consideration. For example, the content of water-soluble calcium salt in the food of the present invention is in an amount suitable to make the concentration of calcium in saliva in the oral cavity to be preferably about 1.0 mM or more, more preferably about 1.5 mM or more, furthermore preferably about 2.0 mM or more, particularly preferably about 2.5 mM or more, and most preferably about 3.0 mM or more, when the food exists in the oral cavity. For example, the content of water-soluble calcium salt in the food of the present invention is in an amount suitable to make the concentration of calcium in saliva in the oral cavity to be preferably about 12 mM or less, more preferably about 6 mM or less, furthermore preferably about 5 mM or less, particularly preferably 4.5 mM or less, and most preferably about 4 mM or less, when the food exists in the oral cavity.

For example, in the case where water-soluble calcium salt (including phosphorylated saccharide calcium) is added in chewing gum, the amount of saliva secreted during chewing for 20 minutes is 20 mL and the molecular weight of calcium is about 40. Therefore, in order to make the concentration of calcium in saliva in the oral cavity to be 1 mM to 15 mM when the food exists in the oral cavity, 0.8 mg to 12 mg of calcium may be contained as an intake per one time (40×1 (mM)×0.02 (L)=0.8 mg, 40×15 (mM)×0.02 (L)=12 mg). Therefore, assuming that the weight of the gum is X g, and the added amount (calculated in terms of calcium) is Y %, then the added amount is decided by the equation: Y(%)={(0.8 to 12 (mg))/(X(g)×1,000)}×100. For example, when the weight of the gum is 2 g, then the added amount in terms of calcium is from 0.04 to 0.6% by weight. For example, when the weight of the gum is 1 g, then the added amount in terms of calcium is from 0.08 to 1.2% by weight. When the weight of the gum is 10 g, then the added amount in terms of calcium is from 0.008 to 0.12% by weight. Even in the case where the weight of the gum is other than the above, the added amount in terms of calcium is calculated in the same manner. Food other than the gum can be designed in the same manner.

The concentration of fluoride in the food of the present invention is preferably adjusted so that the concentration of fluoride ions in the oral cavity is about 0.2 ppm to about 100 ppm, and more preferably about 0.2 ppm to about 1 ppm when the food is used in the oral cavity.

In one embodiment, the concentration of fluoride in the food of the present invention can be arbitrarily set taking the form of the food, the dilution ratio upon eating and the like into consideration. For example, the concentration of fluoride in the food of the present invention is in an amount suitable to make the concentration of fluoride in saliva in the oral cavity to be preferably about 0.01 ppm or more, more preferably about 0.1 ppm or more, furthermore preferably about 0.2 ppm or more, even more preferably about 0.3 ppm or more, particularly preferably about 0.4 ppm or more, and most preferably about 0.5 ppm or more, when the food exists in the oral cavity. The concentration of a fluoride is in an amount suitable to make the concentration of fluoride in saliva in the oral cavity to be preferably about 100 ppm or less, more preferably about 50 ppm or less, furthermore preferably about 10 ppm or less, particularly preferably about 5 ppm or less, and most preferably about 1 ppm or less, when the food exists in the oral cavity.

In the case where the concentration of fluoride is too large, action and effect of a phosphorylated oligosaccharide may be sometimes inhibited, thus making it difficult to obtain a sufficient remineralization effect. In the case where the concentration of fluoride is too small, it is not easy to obtain the tooth improving effect by fluoride.

For example, when fluoride is added into a chewing gum containing a phosphorylated saccharide or a salt thereof, the amount of saliva secreted during chewing for 20 minutes is 20 mL and about 50% to about 60% of the amount is released. Therefore, in order to make the concentration of fluoride in saliva in the oral cavity to be 0.2 to 100 ppm when the food exists in the oral cavity, 0.008 to 4 mg of fluoride may be contained as an intake per one time (20(g)×(0.2 to 100)×10⁻⁶=0.008 to 4 (mg)). Therefore, assuming that the weight of the gum is X g, and the amount of the added fluoride (calculated in terms of fluoride) is Y %, then the added amount is decided by the equation: Y(%)={0.004 to 2 (mg)/(X(g)×1,000)}×100. For example, when the weight of the gum is 2 g, then the added amount in terms of fluoride is from 0.0004 to 0.2% by weight. For example, when the weight of the gum is 1 g, then the added amount in terms of fluoride is from 0.0008 to 0.4% by weight. When the weight of the gum is 10 g, then the added amount in terms of fluoride is from 0.00008 to 0.04% by weight. Even in the case where the weight of the gum is other than the above, the added amount in terms of fluoride is calculated in the same manner. Food other than the gum can be designed in the same manner.

In a specific embodiment, the concentration of the component (1) (i.e. (i) a phosphorylated saccharide calcium salt; or (ii) a combination of phosphorylated saccharide salt other than phosphorylated saccharide calcium salt or phosphorylated saccharide, and water-soluble calcium salt other than phosphorylated saccharide calcium salt) is from 1 mM to 12 mM in terms of the calcium concentration. In this case, the concentration of fluoride is preferably about 0.001 time or more, more preferably about 0.002 time or more, still more preferably about 0.003 time or more, particularly preferably about 0.005 times or more, and most preferably about 0.01 time or more, the concentration of calcium derived from the component (1) in terms of the fluoride concentration. In this specific embodiment, the concentration of fluoride is preferably about 1.5 times or less, more preferably about 1.0 time or less, still more preferably about 0.5 time or less, particularly preferably about 0.1 time or less, and most preferably about 0.05 time or less, the concentration of calcium derived from the component (1) in terms of the fluoride concentration.

In one embodiment, the content of polyphenols in the food of the present invention can be arbitrarily set taking the form of the food, the dilution ratio upon eating and the like into consideration. For example, the content of polyphenols in the food of the present invention is in an amount suitable to make the total concentration of the all polyphenols in saliva in the oral cavity to be preferably about 0.0001% by weight or more, more preferably about 0.0005% by weight or more, still more preferably about 0.001% by weight or more, even more preferably about 0.003% by weight or more, particularly preferably about 0.004% by weight or more, and most preferably about 0.001% by weight or more, when the food exists in the oral cavity. The content of polyphenols in the food of the present invention is in an amount suitable to make the total concentration of the all polyphenols in saliva in the oral cavity to be preferably about 0.1% by weight or less, more preferably about 0.05% by weight or less, and further preferably about 0.01% by weight or less, when the food exists in the oral cavity. In one embodiment, the content of polyphenols in the food of the present invention may be in an amount suitable to make the total concentration of the all polyphenol in saliva in the oral cavity to be preferably about 0.001% by weight or less, more preferably about 0.003% by weight or less, and particularly preferably about 0.001% by weight or less, when the food exists in the oral cavity. In another embodiment, the content of polyphenols in the food may be in an amount suitable to make the total concentration of all the polyphenols in saliva in the oral cavity to be about 0.0001% by weight or less, optionally about 0.005% by weight or less, about 0.002% by weight or less, or about 0.001% by weight or less, when the food exists in the oral cavity.

When the concentration of polyphenols is too high, it is not easy to obtain a sufficient remineralization effect. When no polyphenol is present at all, it becomes slightly easy to react calcium with fluoride. When calcium is reacted with fluoride, calcium fluoride is produced and it becomes impossible to provide a tooth with calcium, and thus the remineralization effect is inhibited.

For example, when a polyphenol is added into a chewing gum, the amount of saliva secreted during chewing for 20 minutes is 20 mL. Therefore, in order to make the concentration of polyphenols in saliva in the oral cavity in to be 0.001% by weight to 0.1% by weight when the food exists in the oral cavity, 0.2 to 20 mg of a polyphenol may be contained as an intake per one time (20 (g)×(0.001 to 0.1)×10⁻²=1 (mg)). Therefore, assuming that the weight of the gum is X g, and the added amount of a polyphenol (calculated in terms of the total amount of polyphenols) is Y %, then the amount is decided by the equation: Y(%)={(0.2 to 20) (mg)/(X(g)×1,000)}×100. For example, when the weight of the gum is 2 g, then the added amount of a polyphenol is from 0.01 to 1% by weight. For example, when the weight of the gum is 1 g, then the added amount of a polyphenol is from 0.02 to 2% by weight. When the weight of the gum is 10 g, then the added amount of a polyphenol is from 0.002 to 0.2% by weight. Even in the case where the weight of the gum is other than the above, the added amount of a polyphenol is calculated in the same manner. Food other than the gum can be designed in the same manner.

Regarding a ratio of fluoride concentration to polyphenol concentration, the amount of polyphenol is preferably 2,000 times or less, more preferably 1,000 times or less, still more preferably 500 times or less, further preferably 200 times or less, particularly preferably 100 times or less, and most preferably 50 times or less, relative to the amount of fluoride. The amount of polyphenol is preferably 1 time or more, more preferably 2 times or more, still more preferably 5 times or more, further preferably 10 times or more, particularly preferably 20 times or more, and most preferably 30 times or more, relative to the amount of fluoride. When the proportion of polyphenol is too high, it is not easy to obtain sufficient remineralization effect.

The content of tea extract in the food of the present invention is preferably adjusted so that the concentrations of fluoride ions and polyphenol in the oral cavity are within the above preferred ranges when the food is used in the oral cavity.

In one embodiment, when the food of the present invention contains a phosphoric acid source compound, the content of the phosphoric acid source compound in the food of the present invention can be arbitrarily set taking the form of the food, the dilution ratio upon eating and the like into consideration. For example, the content of the phosphoric acid source compound in the food of the present invention is in an amount suitable to make the concentration of a phosphoric acid in saliva in the oral cavity to be preferably about 0.1 mM or more, more preferably about 0.5 mM or more, furthermore preferably about 1 mM or more, particularly preferably about 2 mM or more, and most preferably about 2.5 mM or more, when the food exists in the oral cavity. For example, the content of the phosphoric acid source compound in the food of the present invention is in an amount suitable to make the concentration of phosphoric acid in saliva in the oral cavity to be preferably about 10 mM or less, more preferably about 8 mM or less, furthermore preferably about 6 mM or less, particularly preferably about 5 mM or less, and most preferably about 4 mM or less, when the food exists in the oral cavity.

In one embodiment, the content of a phosphoric acid source compound in the food of the present invention can be arbitrarily set taking the form of food, the dilution ratio upon eating and the like into consideration. For example, in the case where phosphoric acid source compound is added into a chewing gum, the amount of saliva secreted during chewing for 20 minutes is 20 mL and the molecular weight of phosphoric acid is about 98. Therefore, in order to make the concentration of phosphoric acid in saliva in the oral cavity to be 0.1 mM to 10 mM when food exists in the oral cavity, 0.0196 mg to 1.96 mg of phosphoric acid may be contained as an intake per one time (98×0.1 (mM)×0.002 (L)=0.0196 mg, 98×10 (mM)×0.002 (L)=1.96 mg). Therefore, assuming that the weight of the gum is X g, and the added amount (calculated in terms of phosphoric acid) is Y %, then the added amount is decided by the equation: Y(%)={(0.0196 to 1.96 (mg))/(X(g)×1,000)}×100. For example, when the weight of the gum is 2 g, then the added amount in terms of phosphoric acid is from 0.00098 to 0.098% by weight. For example, when the weight of the gum is 1 g, then the added amount in terms of phosphoric acid is from 0.00196 to 0.0000196% by weight. When the weight of the gum is 10 g, then the added amount in terms of phosphoric acid is from 0.000196 to 0.00000196% by weight. Even in the case where the weight of the gum is other than the above, the added amount in terms of phosphoric acid is calculated in the same manner. Food other than the gum can be designed in the same manner.

Most preferred ranges of amounts added for a gum with a specific embodiment are summarized below:

Ca/P ratio=about 1 to 2 (desirably about 1.67), this is a value taking about 3.6 mM of phosphoric acid in saliva into consideration, and calcium added/average value in saliva=about 3.6 mM+additive amount of phosphoric acid;

Suitable amount of additive concentration:

Calcium concentration=about 1 to 12 mM, calcium is preferably derived from POs-Ca;

Polyphenol concentration=about 0.001% to 0.1%;

Phosphoric acid concentration=about 0 to 9 mM (desirably about 0.002 to 0.02%);

Fluoride concentration=about 0.5 ppm to 100 ppm (desirably about 0.5 to 1 ppm).

(3c. Method of Eating Food of the Present Invention)

The food of the present invention can be used for any application. The food of the present invention can be used for a healthy human, and a person who needs treatment for early caries lesion.

There is no particular limitation on the amount of intake, the intake frequency and the intake period of the food of the present invention, and the food can be arbitrarily taken.

The amount of intake of the food of the present invention is preferably about 0.1 g or more, more preferably about 0.2 g or more, still more preferably about 0.5 g or more, and further more preferably about 1 g or more, at any one time. There is no particular upper limit of the amount of intake of the food of the present invention. For example, the amount of intake of the food of the present invention is about 1,000 g or less, about 750 g or less, about 500 g or less, about 250 g or less, about 100 g or less, about 50 g or less, about 40 g or less, about 30 g or less, about 20 g or less, about 10 g or less, about 7.5 g or less, about 5 g or less, about 4 g or less, about 3 g or less, about 2 g or less, about 1 g or less or the like, per one time.

The intake frequency of the food of the present invention can be arbitrarily set. For example, the intake frequency for the food of the present invention can be 1 or more times a week, 2 or more times a week, 3 or more times a week, 4 or more times a week, 5 or more times a week, 6 or more times a week, 7 or more times a week, 1 or more times a day, 2 or more times a day, 3 or more times a day, or the like. There is no upper limit on the intake frequency for the food of the present invention. For example, the intake frequency for the food of the present invention can be 3 or less times a day, 2 or less times a day, 1 or less time a day, 7 or less times a week, 6 or less times a week, 5 or less times a week, 4 or less times a week, 3 or less times a week, 2 or less times a week, one or less time a week, or the like.

Intake timing for the food of the present invention may be before meal, after meal or between meals, but is preferably after meal. “Before meal” refers to a time period ranging from a point in time immediately before taking a meal to a point in time of about 30 minutes before taking the meal, “after meal” refers to a time period ranging from a point in time immediately after taking a meal to a point in time of about 30 minutes after taking the meal, and “between meals” refers to a period of time ranging from the time when about 2 or more hours passed after eating meal to the time of about 2 hours or more before the next meal.

The intake period for the food of the present invention can be arbitrarily determined. The food of the present invention can be preferably taken for about 1 day or more, more preferably about 3 days or more, and most preferably about 5 days or more. The intake period for the food of the present invention may be about a month or less, about 2 weeks or less, or about 10 days or less. Since demineralization in the oral cavity can routinely arise, the food of the present invention is preferably taken nearly permanently.

It is preferred that the food of the present invention is not swallowed immediately upon intake, namely, upon eating and is allowed to remain in the oral cavity over a certain period of time. The time, during which the food of the present invention is allowed to remain in the oral cavity, is preferably about 1 minute or more, more preferably about 2 minutes or more, still more preferably about 3 minutes or more, and particularly preferably about 5 minutes or more. In one preferred embodiment, the time is about 10 minutes or more. In a more preferred embodiment, the time is about 15 minutes or more. There is no particular upper limit of the time during which the food of the present invention is allowed to remain in the oral cavity. For example, the time can be about 1 hour or less, about 50 minutes or less, about 40 minutes or less, about 30 minutes or less, about 20 minutes or less, or the like. When the residence time is too short, it is not easy to obtain the remineralization effect.

In the case where the food of the present invention is a chewing gum, a candy, a tablet candy or the like, one piece may be taken at any one time, or plural pieces (for example, 2 to 10 pieces) may be taken at any one time. In the case of taking plural pieces at any one time, the plural pieces may be put and taken in the mouth at a time, or each piece among the plural pieces may be sequentially taken. In the case where the food of the present invention is a chewing gum, it is preferred to continuously chew the gum over a long time. In the case where the food of the present invention is a candy or a tablet candy, it is preferred to suck the food till the last without crunching.

The food of the present invention is usually marketed after packaging. This package can be a commonly used packaging of paper, plastic, cellophane or the like. It is preferred to describe an indication of the intake amount, intake timing, intake method and the like of the food of the present invention (for example, in the case of a gum, “it is preferred to continuously chew two gum pieces for about 20 minutes or more”) on the package. Alternatively, a prescription, in which such an indication is described, may be inserted into the package.

### 4. Cariostatic Oral Composition of the Present Invention

In one embodiment, the cariostatic oral composition of the present invention is a composition containing (1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt; (2) a fluoride; and (3) a polyphenol. In a specific embodiment, it is preferred that this composition contains neither hydroxyapatite fine particles nor a phosphorylated saccharide or a phosphorylated saccharide salt. This cariostatic oral composition can contain calcium phosphate other than hydroxyapatite (for example, calcium monohydrogen phosphate, calcium dihydrogen phosphate and tricalcium phosphate). It is preferred that the composition for the treatment of early caries lesion of the present invention further contains a fluoride or a phosphoric acid source compound. In one embodiment, the cariostatic oral composition of the present invention is preferably a composition for the treatment of early caries lesion.

The cariostatic oral composition of the present invention may be composed only of the materials described above, or may contain materials other than the materials described above. Examples of the other materials that may be contained in the cariostatic oral composition of the present invention include powderedcellulose, starch, water, an antibacterial agent and a sterilizer.

In the case where the cariostatic oral composition of the present invention is a powder, this composition can be produced by mixing (1) (i) a phosphorylated saccharide calcium salt; or (ii) a combination of a phosphorylated saccharide salt other than a phosphorylated saccharide calcium salt or a phosphorylated saccharide, and a water-soluble calcium salt other than a phosphorylated saccharide calcium salt; (2) a fluoride; and (3) a polyphenol, optionally with conventionally known other materials, by a conventionally known method.

In the case where the cariostatic oral composition of the present invention is a liquid, this composition can be produced by adding (1) (i) a phosphorylated saccharide calcium salt; or (ii) a combination of phosphorylated saccharide salt other than phosphorylated saccharide calcium salt or phosphorylated saccharide, and water-soluble calcium salt other than phosphorylated saccharide calcium salt; (2) fluoride; and (3) polyphenol, to a conventionally known solvent, followed bymixing by a conventionally known method.

In one embodiment, the total content of phosphorylated saccharide or salt thereof in the cariostatic oral composition of the present invention can be arbitrarily set taking the form of the oral composition, the dilution ratio upon use, and the like into consideration. For example, the total content of the phosphorylated saccharide or a salt thereof (excluding phosphorylated saccharide calcium) in the cariostatic oral composition of the present invention is in an amount suitable to make the concentration of a phosphorylated saccharide in a mixture of the composition and saliva in the oral cavity to be preferably about 1.0 mM or more, more preferably about 1.5 mM or more, furthermore preferably about 2.0 mM or more, particularly preferably about 2.5 mM or more, and most preferably about 3.0 mM or more, when the composition is used in the oral cavity. For example, the content (total) of the phosphorylated saccharide and salt thereof in the composition of the present invention is in an amount suitable to make the concentration of a phosphorylated saccharide in a mixture of the composition and saliva in the oral cavity to be preferably about 10 mM or less, more preferably about 6 mM or less, furthermore preferably about 5 mM or less, particularly preferably about 4.5 mM or less, and most preferably about 4 mM or less, when the composition is used in the oral cavity.

When used in the present description for the oral composition, “the content is in an amount suitable to make the concentration of it in a mixture of the composition and saliva in the oral cavity to be 1.0 mM or more when the composition is used in the oral cavity” refers to an amount suitable to make the concentration, which is obtained by collecting a liquid produced in the oral cavity during 20 minutes from the start of using the oral composition of the present invention and measuring the concentration of the component in the liquid, to 1.0 mM. The other concentrations will be similarly interpreted. The liquid accumulated in the oral cavity is a mixture of pure saliva, a liquid portion derived from the oral composition, and various solutes derived from the oral composition.

In the case where the oral composition is used in such a form that it acts in the original concentration without being substantially diluted in the oral cavity, as in the case of a dentifrice, a mouth rinsing agent and the like, the total content of a phosphorylated saccharide or a salt thereof in the cariostatic oral composition of the present invention is preferably about 1.0 mM or more, more preferably about 1.5 mM or more, furthermore preferably about 2.0 mM or more, particularly preferably about 2.5 mM or more, and most preferably about 3 mM or more, in terms of the concentration of the phosphorylated saccharide. In this case, for example, the total content of the phosphorylated saccharide or a salt thereof in the cariostatic oral composition of the present invention is preferably about 10 mM or less, more preferably about 6 mM or less, furthermore preferably about 5 mM or less, particularly preferably about 4.5 mM or less, and most preferably about 4 mM or less, in terms of the calcium content. In the case of an oral composition which is intended for use with dilution in the oral cavity, components are added by taking the dilution ratio into consideration. For example, in the case of an oral composition which is intended to be diluted about 20 times, components are added in the concentration of 20 times.

In one embodiment, the content of a water-soluble calcium salt (including phosphorylated saccharide calcium) in the cariostatic oral composition of the present invention can be arbitrarily set taking the form of the oral composition, the dilution ratio upon use and the like into consideration. For example, the content of the water-soluble calcium salt in the cariostatic oral composition of the present invention is in an amount suitable to make the concentration of calcium in a mixture of the composition and saliva in the oral cavity to be preferably about 1.0 mM or more, more preferably about 1.5 mM or more, furthermore preferably about 2.0 mM or more, particularly preferably about 2.5 mM or more, and most preferably about 3.0 mM or more, when the composition is used in the oral cavity. For example, the content of the water-soluble calcium salt in the composition of the present invention is in an amount suitable to make the concentration of calcium in a mixture of the composition and saliva in the oral cavity to be preferably about 10 mM or less, more preferably about 6 mM or less, furthermore preferably about 5 mM or less, particularly preferably 4.5 mM or less, and most preferably about 4 mM or less, when the composition is used in the oral cavity.

In the case where the oral composition is used in such a form that it acts in the original concentration without being substantially diluted in the oral cavity, as in the case of a dentifrice, a mouth rinsing agent and the like, the total content of water-soluble calcium salts in the cariostatic oral composition of the present invention is preferably about 1.0 mM or more, more preferably about 1.5 mM or more, furthermore preferably about 2.0 mM or more, particularly preferably about 2.5 mM or more, and most preferably about 3 mM or more, in terms of the content of the calcium. In this case, for example, the total content of the water-soluble calcium salts in the cariostatic oral composition of the present invention is preferably about 10 mM or less, more preferably about 6 mM or less, furthermore preferably about 5 mM or less, particularly preferably about 4.5 mM or less, and most preferably about 4 mM or less, in terms of the calcium content. In the case of an oral composition which is intended to be used with dilution in the oral cavity, components are added taking the dilution ratio into consideration. For example, in the case of an oral composition which is intended to be diluted about 20 times, components are added in the concentration of 20 times.

The content of fluoride in the cariostatic oral composition of the present invention can be arbitrarily set taking the form of the oral composition, the dilution ratio upon use and the like into consideration. For example, the content of the fluorides in the oral composition of the present invention is in an amount suitable to make the concentration of fluoride in a mixture of the composition and saliva in the oral cavity to be preferably about 0.01 ppm or more, more preferably about 0.1 ppm or more, furthermore preferably about 0.2 ppm or more, even more preferably about 0.3 ppm or more, particularly preferably about 0.4 ppm or more, and most preferably about 0.5 ppm or more, when the composition is used in the oral cavity. The content of the fluoride is in an amount suitable to make the concentration of fluoride in a mixture of the composition and saliva in the oral cavity to be preferably about 100 ppm or less, more preferably about 50 ppm or less, furthermore preferably about 10 ppm or less, particularly preferably about 5 ppm or less, and most preferably about 2 ppm or less, when the composition is used in the oral cavity. These facts are applied to all cariostatic oral compositions of the present invention.

In the case where the oral composition is used in such a form that it acts in the original concentration without being substantially diluted in the oral cavity, as in the case of a dentifrice, a mouth rinsing agent and the like, the total content of fluoride in the cariostatic oral composition of the present invention is preferably about 0.01 ppm or more, more preferably about 0.1 ppm or more, furthermore preferably about 0.2 ppm or more, even more preferably about 0.3 ppm or more, particularly preferably about 0.4 ppm or more, and most preferably about 0.5 ppm or more, in terms of the content of the fluoride. In this case, for example, the total content of fluoride in the cariostatic oral composition of the present invention is preferably about 100 ppm or less, more preferably about 50 ppm or less, furthermore preferably about 30 ppm or less, even more preferably about 10 ppm or less, particularly preferably about 5 ppm or less, and most preferably about 2 ppm or less, in terms of the fluoride content. In the case of an oral composition which is intended to be used with dilution in the oral cavity, components are added taking the dilution ratio into consideration. For example, in the case of an oral composition which is intended to be diluted about 20 times, components are added in the concentration of 20 times.

In the case where the concentration of fluoride is too high, the action and effect of the phosphorylated oligosaccharide may be sometimes inhibited, and thus it is not easy to obtain a sufficient remineralization effect. In the case where the concentration of fluoride is too low, it is not easy to obtain the tooth improvingeffectby fluoride.

In a specific embodiment, the concentration of the component (1) (i.e. (i) a phosphorylated saccharide calcium salt; or (ii) a combination of phosphorylated saccharide salt other than phosphorylated saccharide calcium salt or phosphorylated saccharide, and water-soluble calcium salt other than a phosphorylated saccharide calcium salt) is from 1 mM to 12 mM in terms of the calcium concentration. In this case, the concentration of fluoride is, in terms of the fluoride concentration, preferably about 0.001 time or more, more preferably about 0.002 time or more, still more preferably about 0.003 time or more, particularly preferably about 0.005 time or more, and most preferably about 0.01 time or more, the calcium concentration derived from the component (1). In the case of this specific embodiment, the concentration of fluoride is, in terms of the fluoride concentration, preferably about 1.5 times or less, more preferably about 1.0 time or less, still more preferably about 0.5 time or less, particularly preferably about 0.1 time or less, and most preferably about 0.05 time or less than the calcium concentration derived from the component (1) in terms of the fluoride concentration.

In one embodiment, the content of polyphenols in the oral composition of the present invention can be arbitrarily set taking the form of the oral composition, the dilution ratio upon use and the like into consideration. For example, the content of the polyphenols in the oral composition of the present invention is in an amount suitable to make the total concentration of all polyphenols in a mixture of the composition and saliva in the oral cavity to be preferably about 0.0001% by weight or more, more preferably about 0.0005% by weight or more, furthermore preferably about 0.001% by weight or more, even more preferably about 0.003% by weight or more, particularly preferably about 0.004% by weight or more and most preferably about 0.001% by weight or more, when the composition is used in the oral cavity. The content of the polyphenols in the composition is in an amount suitable to make the total concentration of all polyphenols in a mixture of the composition and saliva in the oral cavity to be preferably about 0.1% by weight or less, more preferably about 0.05% by weight or less, furthermore preferably about 0.01% by weight or less, when the composition is used in the oral cavity. Furthermore, in one embodiment, the content of the polyphenols in the composition is in an amount suitable to make the total concentration of all polyphenols in a mixture of the composition and saliva in the oral cavity to be about 0.001% by weight or less, more preferably about 0.003% by weight or less, particularly preferably about 0.001% by weight or less, when the composition is used in the oral cavity. Furthermore, in one embodiment, the content of the polyphenols in the composition can be in an amount suitable to make the total concentration of all polyphenols in a mixture of the composition and saliva in the oral cavity to be about 0.0001% by weight or less, if necessary about 0.005% by weight or less, about 0.002% by weight or less, or about 0.001% by weight or less, when the composition is used in the oral cavity.

When the concentration of a polyphenol is too large, it is not easy to obtain a sufficient remineralization effect. When polyphenol is not present at all, calcium is slightly likely to react with fluoride. When calcium is reacted with fluoride, calcium fluoride is produced and it becomes impossible to provide a tooth with calcium, and thus the remineralization effect is inhibited.

Regarding the ratio of fluoride concentration to polyphenol concentration, the amount of polyphenol is preferably 2,000 times or less, more preferably 1,000 times or less, still more preferably 500 times or less, further preferably 200 times or less, particularly preferably 100 times or less, and most preferably 50 times or less, the amount of fluoride. The amount of polyphenol is preferably 1 time or more, more preferably 2 times or more, still more preferably 5 times or more, further preferably 10 times or more, particularly preferably 20 times or more, and most preferably 30 times or more, the amount of fluoride. In the case where the proportion of the polyphenol is too high, it is not easy to obtain a sufficient remineralization effect.

The concentration of the tea extract in the oral composition of the present invention is preferably adjusted so that the concentrations of fluoride ions and polyphenols in the oral cavity are within the preferred ranges described above when the composition is used in the oral cavity.

In one embodiment, when the cariostatic oral composition of the present invention contains a phosphoric acid source compound, the concentration of phosphoric acid source compound in the composition can be arbitrarily set taking the form of the oral composition, the dilution ratio upon eating and the like into consideration. The concentration of phosphoric acid source compound in the composition of the present invention is preferably adjusted so that the Ca/P ratio in the oral cavity is within the preferred ranges described above when the composition is used in the oral cavity. In the specific embodiments, the content of phosphoric acid source compound in the composition of the present invention is in an amount suitable to make the concentration of phosphoric acid in a mixture of the composition and saliva in the oral cavity to be preferably about 0.01 mM or more, more preferably about 0.05 mM or more, furthermore preferably about 0.1 mM or more, even more preferably about 0.2 mM or more, particularly preferably about 0.5 mM or more, and most preferably about 1 mM or more, when the composition is used in the oral cavity. The content of the phosphoric acid source compound in the composition of the present invention is in an amount suitable to make the concentration of phosphoric acid in a mixture of the composition and saliva in the oral cavity to be preferably about 15 mM or less, more preferably about 10 mM or less, furthermore preferably about 9 mM or less, particularly preferably about 7 mM or less, and most preferably about 5 mM or less, when the composition is used in the oral cavity.

In the case where the oral composition is used in such a form that it acts in the original concentration without being substantially diluted in the oral cavity, as in the case of a dentifrice, a mouth rinsing agent and the like, the content of phosphoric acid source compounds in the cariostatic oral composition of the present invention is preferably about 0.01 mM or more, more preferably about 0.05 mM or more, furthermore preferably about 0.1 mM or more, even more preferably about 0.2 mM or more, particularly preferably about 0.5 mM or more, and most preferably about 1 mM or more, in terms of the phosphoric acid content. In this case, the content of the phosphoric acid source compounds in the cariostatic oral composition of the present invention is preferably about 15 mM or less, more preferably about 10 mM or less, furthermore preferably about 9 mM or less, particularly preferably about 7 mM or less, and most preferably about 5 mM or less, in terms of the phosphoric acid content.

In another embodiment, the cariostatic oral composition of the present invention can be used in the following manner. Firstly, the cariostatic oral composition of the present invention is applied to a desired tooth surface (for example, a portion of early caries lesion or a healthy portion). This composition is preferably applied to the tooth surface using an appliance such as a contra, a roller or a brush. During or after application of this composition, the composition may be contacted with saliva, and it is possible to take measures to decrease the contact with saliva so as to prevent the applied calcium ions and phosphorylated saccharide ions from flowing out. In the case of taking measures to decrease the contact with saliva, the cariostatic oral composition of the present invention preferably contains a sufficient amount of a phosphoric acid source compound. In this case, for example, it is preferred to remove saliva. It is preferred to continuously take measures to decrease the contact with saliva for about 5 minutes or more, more preferably about 10 minutes or more, and most preferably about 15 minutes or more, from initiation of application of the composition. There is no particular upper limit on the time for taking measures to decrease the contact with saliva. For example, the time can be about 1 hour or less, about 45 minutes or less, about 30 minutes or less, about 25 minutes or less, about 20 minutes or less or the like, from initiation of application of the composition. By taking measures to decrease the contact with saliva, remineralization of early caries lesion can be remarkably accelerated. It is preferred to use an organic substance removing agent before applying the cariostatic oral composition of the present invention to the tooth surface.

In the case where the composition of the present invention is administered into the oral cavity, the composition is preferably allowed to remain in the oral cavity for a certain period of time. The time during which the composition of the present invention is allowed to remain in the oral cavity is preferably about 1 minute or more, and more preferably about 2 minutes or more. The time is still more preferably about 3 minutes or more, and particularly preferably about 5 minutes or more. The time is about 10 minutes or more in one preferred embodiment, and is about 15 minutes or more in a more preferred embodiment. There is no particular upper limit on the time during which the composition of the present invention is allowed to remain in the oral cavity. For example, the time can be about 1 hour or less, about 50 minutes or less, about 40 minutes or less, about 30 minutes or less, or about 20 minutes or less. In the case where the residence time is too short, it is not easy to obtain the remineralization effect.

Examples of the form of the oral composition other than the food include a dentifrice, a mouth rinsing agent (also referred to as mouth wash), a troche, a gel, a spray, a paste, and an ointment, and examples of the dosage form of the pharmaceutical composition include a tablet, a pill, a powder, a solution, a suspension, an emulsion, a granule, and a capsule. It is also possible to use a form such as a wiper cloth obtained by soaking a nonwoven fabric or the like with the solution, or a form such as a cotton swab.

The oral composition of the present invention is usually put on the market after being put in a container or a package. This container can be a container made of plastic or the like which is usually used. This package can be a package of paper, plastic, cellophane or the like, which is usually used. It is preferred to describe an indication of the intake amount, intake timing, intake method and the like for the oral composition of the present invention (for example, in the case of a gum, “it is preferred to continuously chew two gum pieces for about 20 minutes or more”) on the container or package. Alternatively, a prescription, in which such an indication is described, may be inserted into the container or package.

### EXAMPLES

### 1. Phosphorylated Saccharide Calcium Salt Used

A phosphorylated saccharide calcium (POs-Ca) used in the following Tests, Examples and Test Examples refers to a phosphorylated saccharide calcium prepared from potato starch using calcium chloride in place of sodium chloride by the procedure described in Example 1 of Japanese Laid-open Patent Publication No. 8-104696. That is, it is a mixture of phosphorylated saccharide calcium in which 1 to 2 phosphate groups are bonded to an oligosaccharide composed of α-1,4-bonded 2 to 8 glucose in the molecule and calcium is bonded to each of the phosphorylated saccharides. The phosphorylated saccharide calcium is a mixture of those in which one phosphate group is bonded to an oligosaccharide composed of 3, 4 or 5 glucose in the molecule and calcium is bonded to this phosphate group, and those in which 2 phosphate groups are bonded to an oligosaccharide composed of 5, 6, 7 or 8 glucose in the molecule and calcium is bonded to this phosphate group. Herein, a molar ratio of those in which one phosphate groups is bonded to those in which two phosphate groups are bonded is about 8:2. In the following Examples and Test Examples, the salt thus prepared was used. In addition to the present method of using an ion exchange resin, phosphorylated saccharides of various metal salts can be easily prepared by using a method of adding each metal salt after desalination through general electrodialysis. It is noted that regarding a calcium salt of the phosphorylated saccharide, those sold from EZAKI GLICO CO., LTD. as a phosphorylated saccharide calcium can be suitably used.

### 2. Low Polyphenol Content-Tea Extract

A low polyphenol content-tea extract used in the following Tests, Examples and Test Examples was purchased from Mitsui Norin Co., Ltd. The low polyphenol content-tea extract is produced by subjecting a conventional Japanese green tea (sencha-green tea) to hot water extraction at 30° C. to 100° C., and preferably 40° C. to 70° C., and then removing tannin and further removing catechin through activated carbon and a chromatography column, and is a material which can be used as food. The polyphenol content is a value measured by a colorimetric method and the fluoride content is a value measured by an electrode method.

The low polyphenol content-green tea extract 1 used in Test 1 and the like had a fluoride content of 4,650 ppm and a polyphenol content of 7% by weight.

The low polyphenol content-tea extract 2 used in Example 3 and the like had a fluoride content of 3,410 ppm and polyphenols content of 9.85% by weight.

The main components of the polyphenol contained in these low polyphenol content-tea extracts is a mixture of catechin, gallocatechin, catechin gallate, gallocatechin gallate, epicatechin, epigallocatechin, epicatechin gallate and epigallocatechin gallate. The total amount of these polyphenols accounted for about 70% or more of the total weight of the polyphenol.

The material having the same quality as that of these low polyphenol content-tea extracts can be produced by subjecting a Japanese green tea (sencha-green tea) to hot water extractionwith hot water at 30° C. to 100° C., and preferably 40° C. to 70° C., then removing tannin and further removing a polyphenol such as catechin through activated carbon and a chromatography column.

### 3. Formation of Subsurface Demineralized Lesion

In the following Tests, Examples and Test Examples, formation of subsurface demineralized lesion was carried out by the following method. An enamel block (10 mm×10 mm) was cut from the crown of a bovine incisive tooth and then this block was attached to a resin without the oral cavity surface portion. This block was polished with wetted abrasive paper (#1,000 and #2,000) thereby exposing a new flat enamel surface. A portion of the enamel surface was coated with a nail varnish, thereby protecting it from the subsequent demineralization treatment. This portion is a control healthy portion. Subsurface demineralized lesion of the enamel block was formed by immersion into a two-layer system of an 8% methyl cellulose gel and 0.1 M lactate buffer (adjusted to pH 4.7) at 37° C. for 14 days (ten Cate J. M. et al., Caries Res. 40, 400-407, 1996). In such a manner, a bovine tooth piece including a healthy portion and a demineralized portion was prepared.

### 4. Method of TMR

In the following Tests, Examples and Test Examples, the transversal microradiography (TMR) analysis was carried out by the following method. After remineralization or re-demineralization, a thin parallel sections were cut out from the enamel block using a water-cooling type diamond saw. These thin sections were polished so as to obtain a 150 μm thick thin section with a parallel horizontal plane. These thin enamel section were subjected to X-ray photography by Cu—Kα X-ray generated at 20 kV and 20 mA over 13 minutes using a high resolution plate and developed and then microscopic analysis (PW-3830, Philips, The Netherlands) was carried out. In the X-ray photography, various known amounts of aluminum were used simultaneously for X-ray photography as standard substances, and the obtained data were used to make a calibration curve of the amount of calcium. Mineral profiles were drawn from digital images observed by a microscope and then mineral parameters (lesion depth (Ld) and amount of mineral loss (ML)) were calculated by software of Inspektor Research Systems BV (The Netherlands). An average value was calculated per specimen and then statistically analyzed.

(Test 1 and Comparative Tests 1-1 and 1-2: Use of Phosphorylated Saccharide Calcium Salt in Combination with Conventional Tea Extract)

Acceleration of remineralization requires the following two things of:

(1) preventing bonding of calcium to phosphoric acid and insolubilization under a neutral pH condition; and
(2) supplying calcium ions and phosphate ions to a demineralized affected part thereby contributing to crystal growth of hydroxyapatite.

As a result of remineralization by bonding of calcium ions and phosphate ions, hydroxyapatite and hydrogen ions are formed. This reaction is reversible as shown below:

10Ca⁺+6HPO₄⁻+2H₂O [symbol] Ca₁₀(PO₄)₆(OH)₂+8H⁺

Therefore, the remineralization reaction can be monitored by measuring the concentration of calcium ions and the pH. Furthermore, the acceleration effect of the remineralization reaction can be evaluated by utilizing a crystal nucleus of hydroxyapatite (Tanaka, T. et al., Caries Res. 41(4), 327 (2007)).

In order to evaluate an influence of a conventional tea extract or a low polyphenol content-tea extract on mineralization, a change in the pH and calcium ions over time in various remineralization solutions was examined.

Specifically, a remineralization solution with the composition of Comparative Test 1-1 in Table 1 shown below, a remineralization solution with the composition of Comparative Test 1-2 in Table 1, and a remineralization solution with the composition of Test 1 in Table 1 were prepared. The remineralization solution of Comparative Test 1-1 contains a phosphorylated oligosaccharide calcium salt (POs-Ca), but contains note a extract. The remineralization solution of Comparative Test 1-2 contains a phosphorylated oligosaccharide calcium salt and a tea extract. The remineralization solution in Test 1 contains a phosphorylated oligosaccharide calcium salt and a low polyphenol content-tea extract. Any of these solutions contained a phosphoric acid source compound (KH₂PO₄). Any of the supply sources of calcium was a phosphorylated oligosaccharide calcium salt.

When preparing these solutions, firstly, a small amount of a 1N hydrochloric acid solution, a phosphorylated oligosaccharide calcium salt, a phosphoric acid source compound and, in some cases, a green tea extract were added in 50 ml of distilled water and, after mixing, HEPES solution which is a buffer solution was added. Finally, the pH was neutralized by adding 1N potassium hydroxide solution and distilled water was added to make up the solution to 100 ml, and then incubation was initiated at 37° C. at pH 6.5±0.02.

TABLE 1
Composition of remineralization solution

| | Comparative Test 1-1 | | Comparative Test 1-2 | | Test 1 | |
|---|---|---|---|---|---|---|
| | Final | Added | Final | Added | Final | Added |
| | concentration | amount | concentration | amount | concentration | amount |
| Composition | (mM) | (ml) | (mM) | (ml) | (mM) | (ml) |
| Distilled water | — | 76.5 | — | 76.5 | — | 76.5 |
| 2M KCl solution | 100 | 5 | 100 | 5 | 100 | 5 |
| 1M HCl solution | — | 0.05 | — | 0.05 | — | 0.05 |
| 100 mM KH₂PO₄ solution | 3.6 | 3.6 | 3.6 | 3.6 | 3.6 | 3.6 |
| 10% POs-Ca solution*¹ | 6 | 4.8 | 6 | 4.8 | 6 | 4.8 |
| Green tea extract*² | — | — | 0.2% by weight | 0.2 g | — | |
| Low polyphenol content- | — | — | — | — | 0.02% by weight | 0.02 g |
| green tea extract*³ | | | | | | |
| 200 mM HEPES (pH 7.0) | 20 | 10 | 20 | 10 | 20 | 10 |
| 1M KOH solution | — | 0.05 | — | 0.05 | — | 0.05 |
| Total | — | 100 | — | 100 | — | 100 |
| Final fluoride content | 0 ppm | | 0.1 ppm | | 0.9 ppm | |

*¹Solution containing 5% w/w Ca.
*²Camellia extract, manufactured by Taiyo Kagaku Co., Ltd. (powder; polyphenol content 30% by weight).
*³low polyphenol content-green tea extract 1, manufactured by Mitsui Norin Co., Ltd. (powder; fluoride content of 4,650 ppm; polyphenol content of 7%).

While incubating, a change in pH and a change in Ca concentration were measured every 5 minutes for Comparative Tests 1-1 and 1-2, and measured every 1 minute for Test 1. The pH and the Ca concentration at each point in time were measured by an electrode.

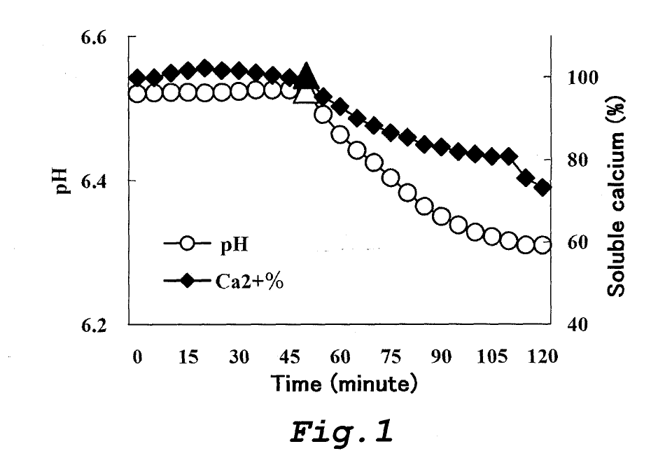

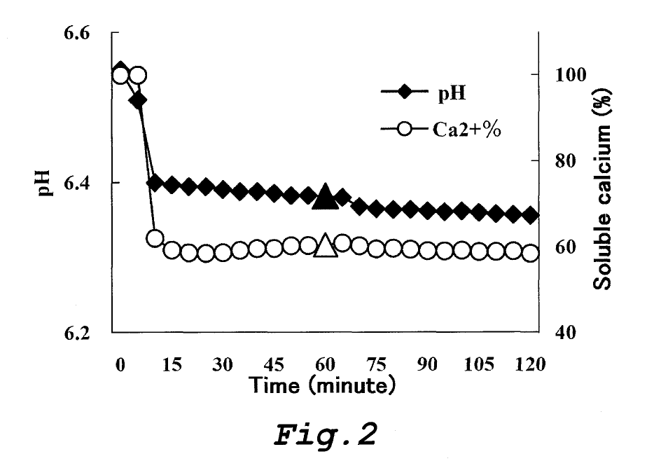

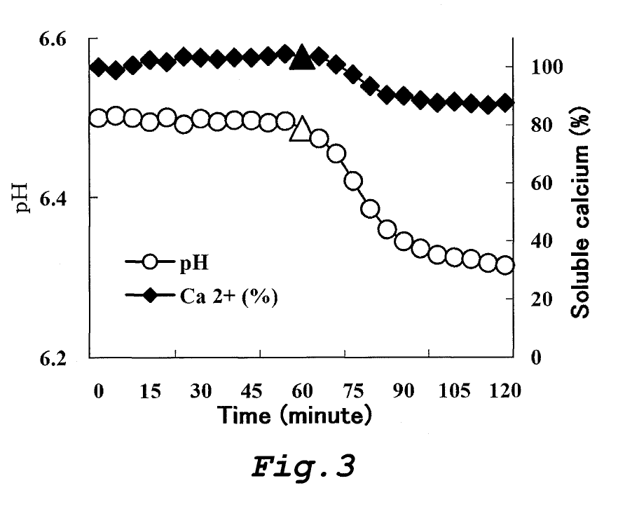

After initiation of incubation, crystal nuclei (100 mg of hydroxyapatite (manufactured by Wako Pure Chemical Industries, Ltd.)) were added to each solution at a time point of 50 minutes for Comparative Test 1-1, at a time point of 60 minutes for Comparative Test 1-2, and at a time point of 12 minutes for Test 1, and then the incubation and measurement were continued until a time point of 120 minutes for Comparative Tests 1-1 and 1-2, and until a time point of 30 minutes for Test 1. The results of Comparative Test 1-1 are shown in FIG. 1, the results of Comparative Test 1-2 are shown in FIG. 2, and the results of Test 1 are shown in FIG. 3.

As shown in FIG. 1 (Comparative Test 1-1), in the case of the remineralization solution containing no tea extract and containing a phosphorylated oligosaccharide calcium, when the incubation was continued without the addition of the crystal nuclei, the calcium concentration and the pH hardly vary, and the solubility of calcium ions was maintained. It was also found that the rapid decrease in the calcium concentration and the pH occurred immediately by addition of the crystal nuclei at 60 minutes later. At 120 minutes after initiation of the incubation, the pH value decreased by 0.21 as compared to those at the initiation time point of the reaction, and 26.7% of calcium ions were insolubilized. This shows that the presence of the crystal nuclei causes acceleration of calcium deposition, and thus remineralization occurs.

On the other hand, as shown in FIG. 2 (Comparative Test 1-2), in the case of the remineralization solution containing both a conventional tea extract and a phosphorylated oligosaccharide calcium salt, the calcium concentration and the pH quickly decreased since immediately after the preparation of the solution and, when they decreased to a certain level, neither a further decrease in calcium concentration nor a further decrease in pH occurred even when the crystal nuclei were added. This shows that calcium is precipitated regardless of the crystal nuclei. At 120 minutes after initiation of the reaction, the pH value decreased by 0.19 as compared to those at the initiation time point of the reaction, and 41.5% of calcium ions were insolubilized.

It was found that from a comparison between FIG. 1 and FIG. 2, a conventional tea extract inhibits the remineralization effect exerted by the phosphorylated oligosaccharide calcium salt. It was found that since a main component of this tea extract is polyphenol, the polyphenol contained in the tea extract (also referred to as tea polyphenol) inhibited remineralization. It is considered that the tea polyphenols inhibit remineralization by adsorption of calcium ions. A conventional tea extract contains a very large amount of fluoride as food, but has a high tea polyphenol content and inhibits remineralization. Therefore, it was found that fluoride in a conventional tea extract cannot be utilized for remineralization.

On the other hand, as shown in FIG. 3 (Test 1), in the case of the remineralization solution containing both low polyphenol content-green tea extract and phosphorylated oligosaccharide calcium salt, when incubation was continued without addition of the crystal nuclei, the calcium concentration and the pH hardly vary, and the solubility of calcium ions was maintained. It was also found that the rapid decrease in calcium concentration and pH occur immediately by the addition of the crystal nuclei at 60 minutes later. At 120 minutes after initiation of the reaction, the pH value decreased by 0.2 as compared to those at the initiation time point of the reaction, and 12.4% of calcium ions were insolubilized. This shows that the presence of the crystal nuclei causes acceleration of calcium deposition, and thus remineralization occurs.

It was found that from these results, the remineralization action exerted by the phosphorylated oligosaccharide calcium salt is hardly inhibited by removing most of the polyphenols from the tea extract. Accordingly, it was found that fluoride contained in the tea extract can be effectively utilized for the improvement of remineralization and acid resistance when most of the polyphenols are removed from the tea extract.

### Example 1

### Confirmation of Effect by Concentration of Fluoride

In the present Example, the concentration of a tea-derived fluoride was examined.

Artificial saliva with each of the compositions of acid resistance tests A to C in Table 2 shown below was prepared. In the present Example, this artificial saliva was used as the remineralization solution. In this case, calcium or phosphoric acid was added and adjusted using a small amount of 1N hydrochloric acid solution and HEPES solution which is a buffer solution. Subsequently, after the pH was neutralized by adding a 1N potassium hydroxide solution, a demineralization treatment was carried out at 36±0.5° C. at pH 6.5±0.02 in the same manner as in the aforementioned “3. Formation of Subsurface Demineralized Lesion”. At a point in time of the demineralization treatment, one forth (¼) of the enamel surface was covered with a nail varnish and the portion of ¾ of the surface had been demineralized. Furthermore, bovine tooth pieces, in which 2/4 of the enamel surface was covered with a nail varnish, were prepared by coating ⅓ of this demineralized portion with the nail varnish. These bovine tooth pieces were put in either of the artificial saliva and then incubated at 37±0.2° C. for 24 hours. Thereafter, the bovine tooth pieces were taken out and the remineralization solution was removed, and then ½ of the remineralized portion was covered with the nail varnish to prepare a bovine tooth piece in which the ¾ of the enamel surface was covered with nail varnish. This bovine tooth piece was immersed in a demineralization gel and incubated at 37° C. for 72 hours thereby re-demineralizing the remineralized portion.

TABLE 2
Composition of artificial salivas

| | Acid resistance test A | | Acid resistance test B | | Acid resistance test C | |
|---|---|---|---|---|---|---|
| | Final | Added | Final | Added | Final | Added |
| | concentration | amount | concentration | amount | concentration | amount |
| Composition | (mM) | (ml) | (mM) | (ml) | (mM) | (ml) |
| Distilled water | — | 76.5 | — | 76.5 | — | 76.5 |
| 2M KCl solution | 100 | 5 | 100 | 5 | 100 | 5 |
| 1M HCl solution | — | 0.05 | — | 0.05 | — | 0.05 |
| 100 mM KH₂PO₄ solution | 3.6 | 3.6 | 3.6 | 3.6 | 3.6 | 3.6 |
| 10% POs-Ca solution*¹ | 6 | 4.8 | 6 | 4.8 | 6 | 4.8 |
| Low polyphenol content- | — | — | — | 4.3 mg | — | 43 mg |
| green tea extract*³ | | | | | | |
| 200 mM HEPES (pH 7.0) | 20 | 10 | 20 | 10 | 20 | 10 |
| 1M KOH solution | | 0.05 | | 0.05 | | 0.05 |
| Total | | 100 | | 100 | | 100 |
| Final fluoride content | 0 ppm | | 0.2 ppm | | 2 ppm | |
| Final polyphenol | 0 ppm | | | | | |
| content | | | | | | |

*¹Solution containing 5% w/w Ca, which is the same as that used in Comparative Test 1-1 and the like.
*³Low polyphenol content-green tea extract 1, manufactured by Mitsui Norin Co., Ltd (powder; fluoride content of 4,650 ppm; polyphenol content of 7%).

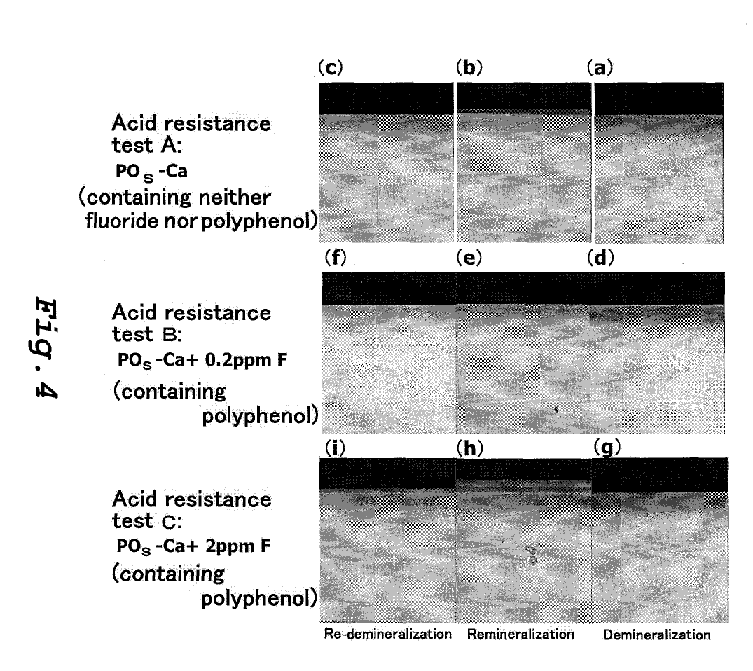

Next, the bovine tooth pieces were recovered and the nail varnishes were peeled and then transversal microradiography(TMR) analysis was carried out in accordance with the aforementioned “4. TMR” to obtain a micrograph of X-ray photography. The results are shown in FIG. 4. The upper panels (a) to (c) of FIG. 4 show the results of an acid resistance test A, the middle panels (d) to (f) of FIG. 4 show the results of an acid resistance test B, and the lower panels (g) to (i) of FIG. 4 show the results of an acid resistance test C. In FIG. 4, (a), (d) and (g) show the results of X-ray photography of the portion subjected to only demineralization treatment. In FIG. 4, (b), (e) and (h) show the results of X-ray photography of the portion subjected to first demineralization, then remineralization treatment, and which was not subsequently subjected to re-demineralization. In FIG. 4, (c), (f) and (i) show the results of X-ray photography of the portion subjected to first demineralization, then a remineralization treatment with various remineralization solutions, and then which was subsequently subjected to re-demineralization. In FIG. 4, a black portion denotes background, and a white portion denotes the portion of a tooth. The upper side is the surface layer side, and the surface layer of the tooth looks white. Slightly black portion of the surface layer of the tooth is the demineralized portion. The higher the intensity of the black color, the higher is the degree of demineralization.

It can be understood from FIG. 4 (b), that the subsurface portion is remineralized to some extent and becomes whitish, and a layer is formed on the surface layer. It can be understood from FIG. 4 (c), that the layer formed in FIG. 4 (b) is removed and the subsurface portion becomes black again, and is demineralized again. Therefore, it is apparent that when the remineralization solution containing a phosphorylated oligosaccharide calcium salt and no tea-derived fluoride is used, remineralization occurs, and a subsurface demineralization occurs again by subsequent re-demineralization.

It can be understood from FIG. 4 (e) that the subsurface portion is remineralized to some extent and becomes white. It can be understood from FIG. 4 (f) that the subsurface portion is white, and is similar to the subsurface portion of FIG. 4 (e) and is hardly demineralized. Therefore, it is apparent that when the remineralization solution containing 0.2 ppm of tea-derived fluoride and a phosphorylated oligosaccharide calcium salt is used, the demineralization of the subsurface portion occurs and the subsurface portion is not highly demineralized even when subjected to a re-demineralization treatment. That is, it was confirmed that acid resistance was obtained.

It can be understood from FIG. 4 (h), that although the subsurface portion becomes slightly white and is remineralized, the degree of remineralization is lower than that in the case of FIG. 4 (e) and a white layer is further formed on the surface layer. It can be understood from FIG. 4 (i) that a portion of the white layer on the surface layer formed in FIG. 4 (h) is removed and also the demineralization of the subsurface portion occurs. Therefore, it is apparent that, although when the remineralization solution containing 2 ppm of fluoride and a phosphorylated oligosaccharide calcium salt is used, a mineral layer is formed on the enamel surface layer, high remineralization of subsurface demineralization is not achieved and a mineral layer on the surface layer is removed by a re-demineralization treatment, and also the demineralization of subsurface portion occurs.

Therefore, it was found that the remineralization effect and the acid resistance effect are obtained by the coexistence of a phosphorylated oligosaccharide calcium and fluoride. Furthermore, fluoride ions have high reactivity and also have high reactivity with calcium ions and, when used in a high concentration, fluoride hardly reaches the target site of the tooth surface in an ionized state and demineralization of the subsurface portion is inhibited, thus fluoride ion is unfavorable. Therefore, it was found that fluoride ions are needed to be properly set and the fluoride concentration is preferably a low concentration of about 0.2 ppm.

(Tests 2-1 and 2-2 and Comparative Tests 2-1 and 2-2: Examination of Polyphenol Concentration)

By varying the amount of low polyphenol content-green tea extract to be added to a remineralization solution, the concentration of polyphenols in the remineralization solution was changed to 0% by weight, 0.0011% by weight, 0.0017% by weight or 0.0022% by weight, and thus a relationship between the polyphenol content and the obtained remineralization effect was confirmed.

Specifically, a remineralization solution with the composition in Table 3 shown below was prepared. All of these solutions contained a phosphoric acid source compound (KH₂PO₄). In all cases, supply sources of calcium was a phosphorylated oligosaccharide calcium salt. In preparing these solutions, firstly, a small amount of a 1N hydrochloric acid solution, a phosphorylated oligosaccharide calcium salt, a phosphoric acid source compound and a low polyphenol content-green tea extract were added to 50 ml of distilled water and mixed, and then HEPES solution which is a buffer solution was added. Finally, the pH was neutralized by adding 1N potassium hydroxide solution and distilled water was added to make up the solution to 100 ml, and then incubation was initiated at 37° C. at pH 6.5±0.02.

TABLE 3
Composition of remineralization solution

| | Comparative | | | | | | Comparative | |
|---|---|---|---|---|---|---|---|---|
| | Test 2-1 | | Test 2-1 | | Test 2-2 | | Test 2-2 | |
| | Final | Added | Final | Added | Final | Added | Final | Added |
| | concentration | amount | concentration | amount | concentration | amount | concentration | amount |
| Composition | (mM) | (ml) | (mM) | (ml) | (mM) | (ml) | (mM) | (ml) |
| Distilled water | — | 76.5 | — | 76.5 | — | 76.5 | — | 76.5 |
| 2M KCl solution | 100 | 5 | 100 | 5 | 100 | 5 | 100 | 5 |
| 1M HCl solution | — | 0.05 | — | 0.05 | — | 0.05 | — | 0.05 |
| 100 mM KH₂PO₄ solution | 3.6 | 3.6 | 3.6 | 3.6 | 3.6 | 3.6 | 3.6 | 3.6 |
| 10% POs-Ca solution*¹ | 6 | 4.8 | 6 | 4.8 | 6 | 4.8 | 6 | 4.8 |
| Low polyphenol content- | — | — | — | 15.7 mg | — | 24.3 mg | — | 31.4 mg |
| green tea extract*³ | | | | | | | | |
| 200 mM HEPES (pH 7.0) | 20 | 10 | 20 | 10 | 20 | 10 | 20 | 10 |
| 1M KOH solution | | 0.05 | | 0.05 | | 0.05 | | 0.05 |
| Total | | 100 | | 100 | | 100 | | 100 |
| Final fluoride content | 0 ppm | | 0.5 ppm | | 0.5 ppm | | 1.0 ppm | |
| Final polyphenol | 0% | | 0.0011% | | 0.0017% | | 0.0022% | |
| content | | | | | | | | |

*¹Solution containing 5% w/w Ca, which is the same as that used in Comparative Test 1-1.
*³Low polyphenol content-green tea extract 1, manufactured by Mitsui Norin Co., Ltd (powder; fluoride content of 4,650 ppm; polyphenol content of 7%).

While incubating, a change in Ca concentration was measured every 5 minutes. The Ca concentration at each time point was measured by an electrode.

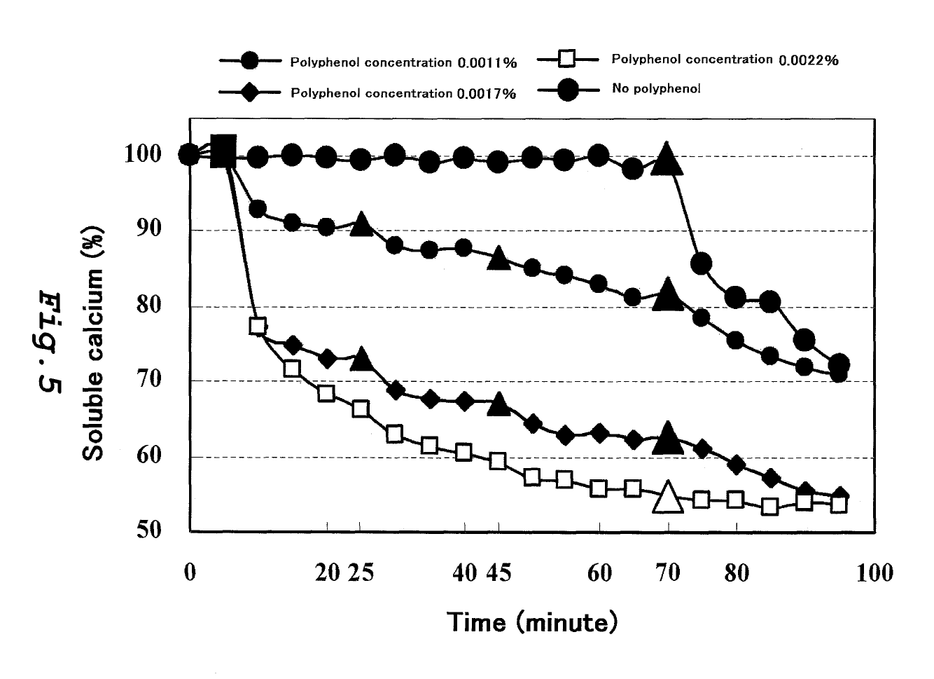

After initiation of the incubation, crystal nuclei (100 mg of hydroxyapatite (manufactured by Wako Pure Chemical Industries, Ltd.)) were added to each solution at a time point of 70 minutes for Comparative Tests 2-1 and 2-2, and at three time points of 25 minutes, 45 minutes and 70 minutes for Tests 2-1 and 2-2, and then the incubation and measurements were continued until a time point of 95 minutes. The results are shown in FIG. 5.

As shown in FIG. 5, in the case of the remineralization solution containing no tea extract and containing phosphorylated oligosaccharide calcium, when the incubation was continued without addition of the crystal nuclei, the calcium concentration and the pH hardly vary, and the solubility of calcium ions was maintained. It was also found that a rapid decrease in the calcium concentration occurs immediately after the addition of the crystal nucle 70 minutes later. At 120 minutes after the initiation of the reaction, about 30% of calcium ions were insolubilized as compared with those at the initiation time point of the reaction.

On the other hand, in the case where the polyphenol concentration was 0.0011% by weight and 0.0017% by weight, the calcium concentration decreased to some extent at a time point of about 10 minutes from the initiation of the incubation even without addition of the crystal nuclei. However, the calcium concentration was further decreased by the addition of the crystal nuclei. Therefore, it was found that when the polyphenol concentration is 0.0017% by weight or less, the remineralization effect of the phosphorylated oligosaccharide calcium salt is hardly inhibited.

Furthermore, in the case where the polyphenol concentration was 0.0022% by weight, the calcium concentration rapidly decreased at a time point of about 10 minutes from the initiation of the incubation even without addition of the crystal nuclei and, thereafter, calcium concentration also gradually decreased. A decrease in the calcium concentration was hardly recognized even after the crystal nuclei were added. This revealed that when the polyphenol concentration is 0.0022% by weight or more, the polyphenol strongly inhibits the remineralization effect of the phosphorylated oligosaccharide calcium salt.

Therefore, it was found that when the content of the polyphenols which inhibits remineralization is decreased, applicability of the tea-derived fluoride is enhanced. It was found that using the phosphorylated oligosaccharide calcium salt, the low-concentration polyphenol and the low-concentration fluoride in combination prevents insolubilization due to the reaction of fluoride ions with calcium ions and thus calcium ions and fluoride ions are efficiently supplied to the target tooth surface.

### Examples 2-1 to 2-3 and Comparative Examples 2-1 to 2-3

### Confirmation of Remineralization Effect at Various Fluoride Concentrations

### (i) Examination of the Influence of Fluoride Concentration

The effect of using a low-concentration fluoride and POs-Ca contained in a tea extract in combination was compared with the effect of using a low-concentration fluoride contained in a tea extract and CaCl₂ in combination. Furthermore, the concentration of a tea extract added, at which the remineralization effect of POs-Ca was not inhibited and also acid resistance of fluoride was obtained, was examined.

In the present Examples and Comparative Examples, bovine toothpieces subjected to a demineralization treatment in the same manner as in “3. Formation of Subsurface Demineralized Lesion” were used. These bovine tooth pieces were bovine tooth pieces in which the portion of ¼ of the enamel surface was a healthy portion and covered with a nail varnish, and also the proton of ¾ of the surface was demineralized and ⅓ of the demineralized portion was covered with a nail varnish.

On the first day, (1) firstly, these tooth pieces were incubated in various remineralization solutions (pH 6.5) shown in Table 4 to Table 6 shown below at 37° C. for 2 hours. (2) Then the tooth pieces were washed with distilled water and moisture was removed, and then incubated in a demineralization solution (Ca concentration of 1.5 mM; P concentration of 0.9 mM; pH 5) shown in Table 7 shown below at 37° C. for 2 hours. (3) The tooth pieces were washed with distilled water and moisture was removed, and then incubated in newly prepared various remineralization solutions shown in Table 4 to Table 6 at 37° C. for 2 hours. In step (3), a new remineralization solution with the same composition as in step (1) was used in each test. After completion of a cycle of steps (1) to (3), the tooth pieces were taken out and kept in an aqueous solution containing 1.5 mM Ca, 5 mM P and 0.9% NaCl at 37° C. A cycle including steps (1) to (3) was carried out in 7 cycles.

In this 7 day treatment period, remineralization was carried out over 7 days, for 4 hours per day and thus remineralization was carried out for 28 hours in total, and demineralization was carried out for 7 days, for 2 hours per day and thus demineralization was carried out for 14 hours in total.

For comparison, a test of subjecting to a remineralization treatment only, for 24 hours was carried out with respect to a combination of 0.5 ppm of fluoride and POs-CaCl₂ (Example 2-4; a remineralization solution with the same composition as in Example 2-2 was used), and a combination of 0.5 ppm of fluoride and CaCl₂ (Comparative Example 2-8; a remineralization solution with the same composition as in Comparative Example 2-3 was used).

TABLE 4
Composition of remineralization solution
In the case of using POs-Ca

| | | | | | | | Comparative | |
|---|---|---|---|---|---|---|---|---|
| | Example 2-1 | | Example 2-2 | | Example 2-3 | | Example 2-1 | |
| | Final | Added | Final | Added | Final | Added | Final | Added |
| | concentration | amount | concentration | amount | concentration | amount | concentration | amount |
| Composition | (mM) | (ml) | (mM) | (ml) | (mM) | (ml) | (mM) | (ml) |
| Distilled water | — | 50 | — | 50 | — | 50 | — | 50 |
| 2M KCl | 100 | 5 | 100 | 5 | 100 | 5 | 100 | 5 |
| 1M HCl | — | 0.05 | — | 0.05 | — | 0.05 | — | 0.05 |
| 100 mM KH₂PO₄ | 3.6 | 3.6 | 3.6 | 3.6 | 3.6 | 3.6 | 3.6 | 3.6 |
| 10% POs-Ca*¹ | 6 | 4.8 | 6 | 4.8 | 6 | 4.8 | 6 | 4.8 |
| Tea extract solution*² | 1 | 1 | 0.5 | 0.5 | 0.2 | 0.2 | — | — |
| 200 mM HEPES | 20 | 10 | 20 | 10 | 20 | 10 | 20 | 10 |
| Distilled water and 1M | adjusted to pH 6.5 | | adjusted to pH 6.5 | | adjusted to pH 6.5 | | adjusted to pH 6.5 | |
| HCl*³ | | | | | | | | |
| Total | | 100 ml | | 100 ml | | 100 ml | | 100 ml |
| Final fluoride content | 1.0 ppm | — | 0.5 ppm | — | 0.2 ppm | — | 0 ppm | — |

*¹Solution containing 5% w/w Ca, which is the same as that used in Comparative Test 1-1 and the like.
*²A solution obtained by dissolving 0.22 g of low polyphenol content-green tea extract 1, manufactured by Mitsui Norin Co., Ltd (powder; fluoride content of 4,650 ppm; polyphenol content of 7%) in 10 mL distilled water. The fluoride concentration of this solution is 100 ppm.
*³After mixing the materials described above, distilled water was added to make 90 ml, and then the pH was adjusted to 6.5 with 1M HCl and distilled water was added to make the total amount to be 100 ml.

TABLE 5
In the case of using CaCl₂

| | Comparative | | Comparative | | Comparative | | Comparative | |
|---|---|---|---|---|---|---|---|---|
| | Example 2-2 | | Example 2-3 | | Example 2-4 | | Example 2-5 | |
| | Final | Added | Final | Added | Final | Added | Final | Added |
| | concentration | amount | concentration | amount | concentration | amount | concentration | amount |
| Composition | (mM) | (ml) | (mM) | (ml) | (mM) | (ml) | (mM) | (ml) |
| Distilled water | — | 50 | — | 50 | — | 50 | — | 50 |
| 2M KCl | 100 | 5 | 100 | 5 | 100 | 5 | 100 | 5 |
| 1M HCl | — | 0.05 | — | 0.05 | — | 0.05 | — | 0.05 |
| 100 mM KH₂PO₄ | 0.9 | 0.9 | 0.9 | 0.9 | 0.9 | 0.9 | 0.9 | 0.9 |
| 100 mM CaCl₂ | 1.5 | 1.5 | 1.5 | 1.5 | 1.5 | 1.5 | 1.5 | 1.5 |
| Tea extract solution*² | 1 | 1 | 0.5 | 0.5 | 0.2 | 0.2 | 10 | 10 |
| 200 mM HEPES | 20 | 10 | 20 | 10 | 20 | 10 | 20 | 10 |
| Distilled water and 1M | adjusted to pH 6.5 | | adjusted to pH 6.5 | | adjusted to pH 6.5 | | adjusted to pH 6.5 | |
| HCl*³ | | | | | | | | |
| Total | | 100 ml | | 100 ml | | 100 ml | | 100 ml |
| Final fluoride content | 1.0 ppm | — | 0.5 ppm | — | 0.2 ppm | — | 10 ppm | — |

*²A solution obtained by dissolving 0.22 g of low polyphenol content-green tea extract 1, manufactured by Mitsui Norin Co., Ltd (powder; fluoride content of 4,650 ppm; polyphenol content of 7%) in 10 mL distilled water. The fluoride concentration of this solution is 100 ppm.
*³After mixing the materials described above, distilled water was added to make 90 ml, and then the pH was adjusted to 6.5 with 1M HCl and distilled water was added to make the total amount to be 100 ml.

TABLE 6
Composition of remineralization solution
In the case of using neither POs-Ca nor CaCl₂

| | Comparative | | | | Comparative | | | |
|---|---|---|---|---|---|---|---|---|
| | Example 2-6 | | | | Example 2-7 | | | |
| | Final | | | | Final | | | |
| | concen- | | Added | | concen- | | Added | |
| | tration | | amount | | tration | | amount | |
| Composition | (mM) | | (ml) | | (mM) | | (ml) | |
| Distilled water | — | | 50 | | — | | 50 | |
| 2M KCl | 100 | | 5 | | 100 | | 5 | |
| 1M HCl | — | | 0.05 | | — | | 0.05 | |
| 100 mM KH₂PO₄ | 0.9 | | 0.9 | | 0.9 | | 0.9 | |
| 100 mM CaCl₂ | — | | — | | — | | — | |
| Tea extract | 10 | | 10 | | 0.5 | | 0.5 | |
| solution*² | | | | | | | | |
| 200 mM HEPES | 20 | | 10 | | 20 | | 10 | |
| Distilled water | adjusted to pH 6.5 | | | | adjusted to pH 6.5 | | | |
| and 1M HCl*³ | | | | | | | | |
| Total | | | 100 | ml | | | 100 | ml |
| Final fluoride | 10 | ppm | — | | 0.5 | ppm | — | |
| content | | | | | | | | |

*²A solution obtained by dissolving 0.22 g of low polyphenol content-green tea extract 1, manufactured by Mitsui Norin Co., Ltd (powder; fluoride content of 4,650 ppm; polyphenol content of 7%) in 10 mL distilled water. The fluoride concentration of this solution is 100 ppm.
*³After mixing the materials described above, distilled water was added to make up to 90 ml, and then the pH was adjusted to 6.5 with 1M HCl and distilled water was added to make up the total amount to 100 ml.

TABLE 7
Composition of demineralization solution

| | | |
|---|---|---|
| | | Added |
| | Final concentration | amount |
| Composition | (mM) | (ml) |

| | | |
|---|---|---|
| Distilled water | — | 50 |
| 2M KCl | 100 | 5 |
| 1M HCl | — | 0.05 |
| 100 mM KH₂PO₄ | 0.9 | 0.9 |
| 100 mM CaCl₂ | 1.6 | 1.6 |
| 200 mM HEPES | 20 | 10 |

| | |
|---|---|
| Distilled water | adjusted to pH 5.0 |
| and 1M HCl*³ | |

| | | |
|---|---|---|
| Total | 100 | ml |

*³After mixing the materials described above, distilled water was added to make 90 ml, and then the pH was adjusted to 6.5 with 1M HCl and distilled water was added to make the total amount to be 100 ml.

After completion of these treatments, the bovine tooth pieces were recovered and the nail varnish were peeled, and then transversal microradiography (TMR) analysis was carried out in accordance with the aforementioned “4. TMR” to obtain a micrograph of X-ray photography. Mineral profiles were drawn, and then lesion depths (Ld) and amounts of mineral loss (ML) were calculated.

A recovery rate of an amount of mineral loss (%) and a recovery rate of a lesion depth (%) were calculated from the obtained mineral profile. Assuming that the amount of mineral loss (mineral loss) of the demineralized portion was 100% loss, the recovery rate of mineral of the remineralized portion was calculated based on the following equation:

[{(Amount of mineral loss of demineralized portion)−(Amount of mineral loss of remineralized portion)}/(Amount of mineral loss of demineralized portion)]×100=Recovery rate(%)

It is noted that this calculation method is applied to the calculation of all other recovery rates.

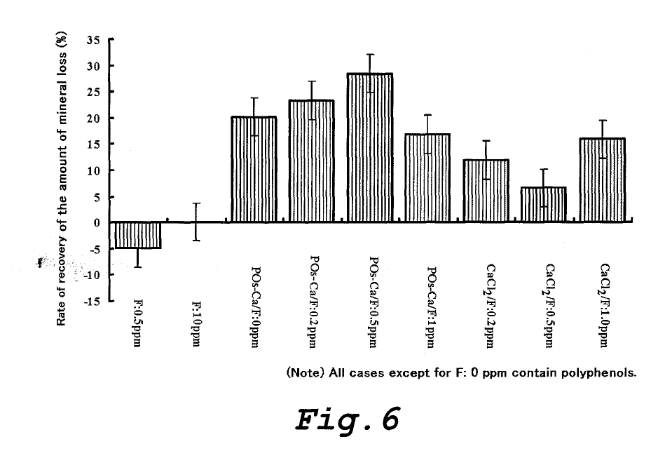

A graph of the recovery rate of an amount of mineral loss (%) is shown in FIG. 6 and, in FIG. 6, only the results in the case of cycling for 7 days are shown.

In FIG. 6, in the case where neither POs-Ca nor CaCl₂ was used and only a tea-derived fluoride was contained, the amount of mineral loss was hardly recovered and was rather decreased. By using a tea-derived fluoride in combination with POs-Ca, the recovery rate of the amount of mineral loss tend to be improved. In the case where a tea-derived fluoride and CaCl₂ were used, the recovery rate of the amount of mineral loss was lower than the case of using a tea-derived fluoride and POs-Ca.

Regarding a tea extract+CaCl₂ in the case where the fluoride concentration was 0.5 ppm, when the case of cycling for 7 days was compared with the case of performing only remineralization, a recovery rate of a lesion depth was low in the case of cycling. This reveals that remineralization by a tea extract+CaCl₂ does not endure a demineralization treatment and the quality of remineralization is poor.

From the test results described above, it was considered that the remineralization effect exerted by a tea extract+POs-Ca is most suitable at a fluoride concentration of about 0.5 ppm. From these facts, when these are actually added in a gum, it was considered desirable to design the fluoride concentration in the food to make the fluoride concentration in the oral cavity about 0.5 ppm.

(ii) Comparison Under Conditions of 0.5 ppm of Fluoride

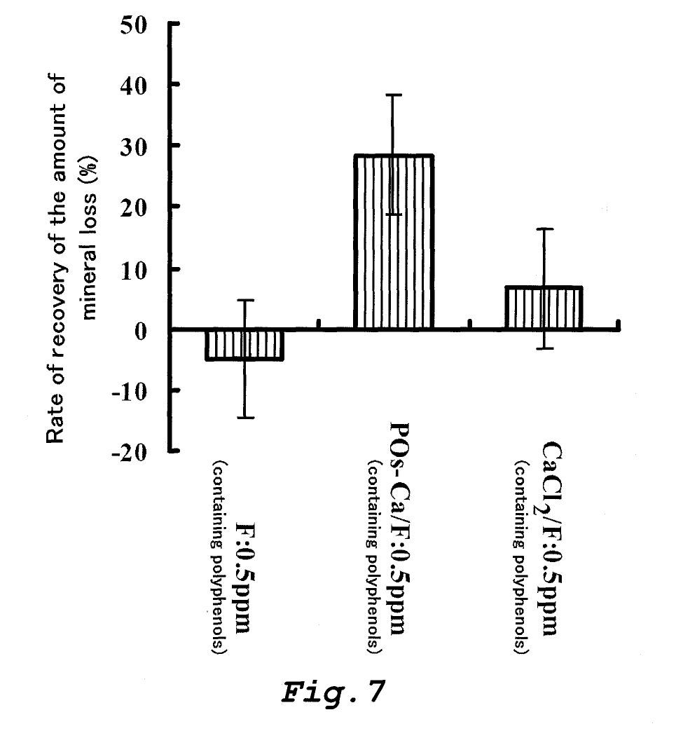

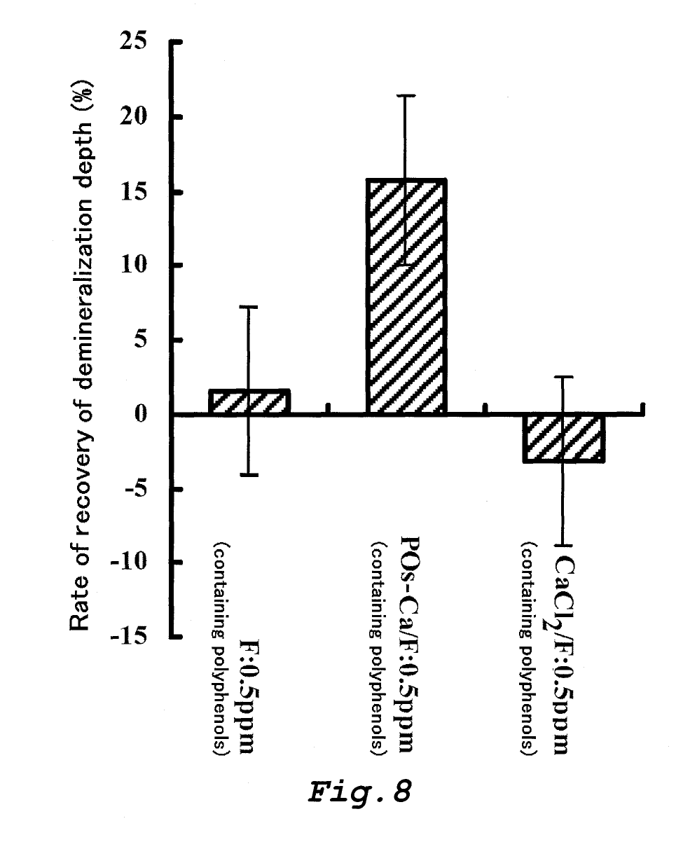

Among the results described above, regarding the results in the case of only 0.5 ppm of fluoride (containing polyphenol), 0.5 ppm of fluoride+POs-Ca (containing polyphenol), or 0.5 ppm of fluoride+CaCl₂ (containing polyphenol), a recovery rate of an amount of mineral loss (%) is shown in FIG. 7 and a recovery rate of a lesion depth (%) is shown in FIG. 8.

It can be understood from FIG. 7 that although a recovery of the amount of mineral loss is scarcely obtained in the cases of fluoride only (containing polyphenol) and 0.5 ppm of fluoride+CaCl₂ (containing polyphenol), a very high recovery of the amount of mineral loss is obtained in the case of 0.5 ppm of fluoride+POs-Ca (containing polyphenol).

It can be understood from FIG. 8 that although a recovery of the lesion depth is scarcely obtained in the cases of fluoride only (containing polyphenol) and 0.5 ppm of fluoride+CaCl₂ (containing polyphenol), a very high recovery of the lesion depth is obtained in the case of 0.5 ppm of fluoride+POs-Ca (containing polyphenol).

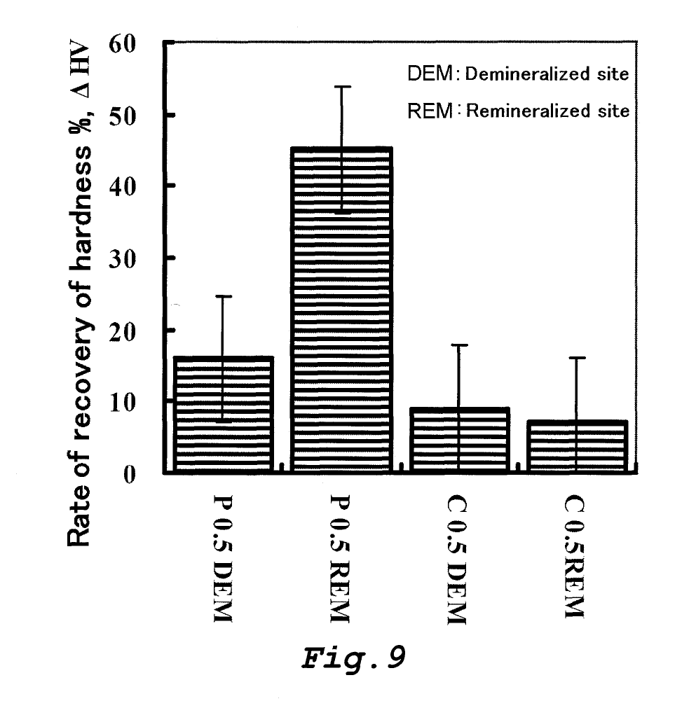

In the cases of 0.5 ppm of fluoride+POs-Ca (containing polyphenol) and 0.5 ppm of fluoride+CaCl₂ (containing polyphenol), hardness of a dental piece of the demineralized portion and that of the remineralized portion were measured by Microhardness (Vickers method). The hardness (ΔHV) of the demineralized portion and that of the remineralized portion are shown in FIG. 9. It was found that, from the results of FIG. 9, although the hardness is recovered by remineralization in the case of 0.5 ppm of fluoride+POs-Ca (containing polyphenol), the hardness is not recovered even by remineralization in the case of 0.5 ppm of fluoride+CaCl₂ (containing polyphenol).

Thus, it was found that very high remineralization effect and a recovery of hardness can be obtained by using fluoride, POs-Ca and a polyphenol in combination.

### Example 3 and Comparative Examples 3-1 and 3-2

### Preparation of Gum

From the above test results of the remineralization test in the artificial saliva, it was considered that both the remineralization effect and the recovery effect of the hardness could be obtained when 0.5 ppm of fluoride is eluted into the saliva. The formulation of a pellet gum was designed taking this fact into consideration.

In accordance with the method usually carried out in the art, materials described in the formulation in Table 8 below were used as materials of a center gum and were sugar coated as ordinary to produce a pellet gum. The gum of Example 3 was a gum containing POs-Ca+F+polyphenol, the gum of Comparative Example 3-1 was a gum which contains POs-Ca but contains neither fluoride nor polyphenol, and the gum of Comparative Example 3-2 was a control gum containing neither POs-Ca nor F nor a polyphenol. The weight per gum pellet was about 1.5 g on average, the weight of center gum was about 1.0 g on average, and the weight of the sugar coating portion was about 0.5 g on average.

TABLE 8
Formulation of center gum

| | | Comparative | Comparative |
|---|---|---|---|
| | | Example | Example |
| | Example 3 | 3-1 | 3-2 |
| | POs-Ca + F | POs-Ca(+) | POs-Ca(−)F(−) |
| | Added amount | Added amount | Added amount |
| Material | (% by weight) | (% by weight) | (% by weight) |
| Gum base | 35 | 35 | 35 |
| POs-Ca*¹ | 2.5 | 2.5 | 0 |
| Low polyphenol | 1.2 | 0 | 0 |
| content-tea extract*² | | | |
| Xylitol | 57.3 | 58.5 | 61 |
| Glycerin | 1 | 1 | 1 |
| Mint oil | 3 | 3 | 3 |
| Total | 100.0 | 100.0 | 100.0 |

*¹Raw material containing 5% w/w Ca; powder.
*²A material which was obtained by diluting a low polyphenol content-tea extract 2 having a F content of 3,410 ppm and polyphenols content of 9.85%, 34.1 folds with distilled water thereby adjusting the F content to 100 ppm and the polyphenol content to 0.289%.

### Example 4

### Remineralization Test

Using the gums produced in above mentioned Example 3 and Comparative Examples 1-1 and 1-2 and a commercially available tea-derived fluoride-containing gum, the remineralization effect and resistance against demineralization were examined. This commercially available tea-derived fluoride-containing gum is a gum with a raw material containing 1,500 ppm or more of fluoride and 18% of polyphenols in an amount of 0.88% per 2.5 g of the formulation of one gum stick.

Bovine tooth pieces with early caries lesion formed thereon were prepared in accordance with “3. Formation of Subsurface Demineralized Lesion”. The reaction areas were set for four areas: a healthy portion, a demineralized portion, a remineralized portion and a re-demineralized portion.

The procedure of a remineralization test will be described in detail below.

(1) For various gums, 4 replicate gum pellets for each were immersed in 40 mL of a solution A for extraction (Ca/P=0.3; pH 6.5) at 37° C. for 20 minutes thereby extracting components of the gum to obtain extraction liquid A.

(2) A bovine tooth piece with early caries lesion formed thereon was immersed in the extraction liquid A and incubated at 37° C. for 2 hours.

(3) This tooth piece was washed with distilled water.

(4) 40 mL of a demineralization solution B was prepared. The demineralization solution B was adjusted to pH 5 with HEPES buffer base; lactate at 37° C.

(5) The tooth piece which was immersed in the solution A in step (2) and washed with distilled water in step (3), was immersed in the demineralization solution B prepared in step (4) and then incubated at 37° C. for 2 hours.

(6) For various gums, 4 replicate gum pellets for each were immersed in a 40 mL of a solution A for extraction (Ca/P=0.3; pH 6.5) at 37° C. for 20 minutes thereby extracting components of the gum to obtain an extraction liquid A again.

(7) The tooth piece immersed in step (5) was immersed in the extraction liquid A prepared in step (6) and then incubated at 37° C. for 2 hours.

(8) The tooth piece was taken out and washed with distilled water.

(9) The tooth piece in step (8) was immersed in a preservation solution C and stored in an incubator at 37° C. until the next day.

A treatment was carried out by steps (1) to (9) for 5 days. After treatment for 5 days, a region of ½ of the remineralized portion was covered with a nail varnish thereby protecting from the subsequent demineralization treatment. This tooth piece was subjected to a re-demineralization treatment by immersing in a demineralization solution described in Table 9 shown below and incubating at 37° C. for 72 hours. These treatment conditions are conditions where a variation in the pH in the oral cavity is reflected.

TABLE 9
Compositions of solution A for extraction, demineralization solution B and preservation solution C

| | Solution A | | Demineralization | | Preservation | |
|---|---|---|---|---|---|---|
| | for extraction | | solution B | | solution C | |
| | Final | Added | Final | Added | Final | Added |
| | concentration | amount | concentration | amount | concentration | amount |
| Composition | (mM) | (ml) | (mM) | (ml) | (mM) | (ml) |
| Distilled water | — | 500 | — | 500 | — | — |
| 2M KCl | 100 | 50 | 100 | 50 | — | — |
| 1M HCl | — | 0.5 | — | 0.5 | — | — |
| 100 mM KH₂PO₄ | 3.6 | 36 | 3.6 | 36 | 5 | 5 |
| 1.80% NaCl | — | — | — | — | — | 50 |
| 100 mM CaCl₂ | 1 | 10 | 1 | 10 | 1.5 | 1.5 |
| 1M Lactic acid | — | — | — | — | — | 100 μl |
| 200 mM HEPES | 20 | 100 | 20 | 100 | 20 | 10 |
| Distilled water | Adjusted to pH 6.5 | | Adjusted to pH 5 | | Adjusted to pH 6 | |
| and 1M HCl, 1M KOH | with 1M HCl | | with 1M lactic acid | | with 1M lactic acid | |
| or 1M lactic acid*¹ | | | | | | |
| Total | | 1 L | | 1 L | | 100 ml |

*¹After mixing the materials described above, distilled water was added to make 90 ml, and then the pH was adjusted to 6.5 with 1M HCl and distilled water was added to make the total amount to be 100 ml.

After completion of these treatments, the bovine tooth pieces were recovered and the nail varnishes were peeled, and then micrographs of X-ray photography were obtained by transversal microradiography (TMR) analysis in accordance with the aforementioned “4. TMR”. Mineral profiles were drawn and then lesion depths (Ld) and amounts of mineral loss (ML) were calculated.

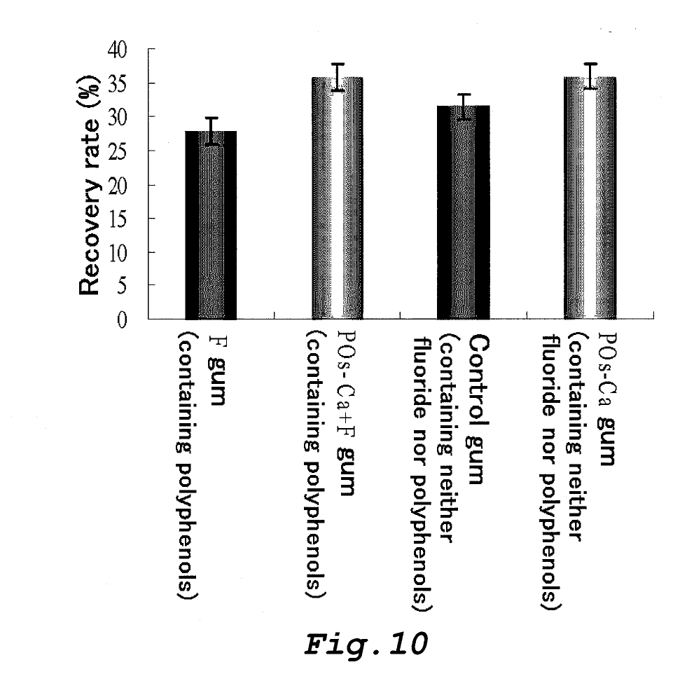

The results obtained from the mineral profiles of TMR analysis, recovery rates of the amounts of mineral loss and the recovery rates of the lesion depth by the remineralization treatment for 5 days were calculated. The results are shown in Table 10 below and FIG. 10 to FIG. 13.

TABLE 10

| | | | POs-Ca + F | | POs-Ca | | Cont | |
|---|---|---|---|---|---|---|---|---|
| | F Gum | | Gum | | Gum | | Gum | |
| | ML % | Ld % | ML % | Ld % | ML % | Ld % | ML % | Ld % |
| REM % | 27.8 | 14.3 | 35.7 | 11.5 | 35.9 | 8.4 | 31.4 | −1.7 |
| ReDEM % | 4.2 | 4.9 | 13.7 | 7.5 | 7.1 | 4.2 | 5.0 | 0.3 |

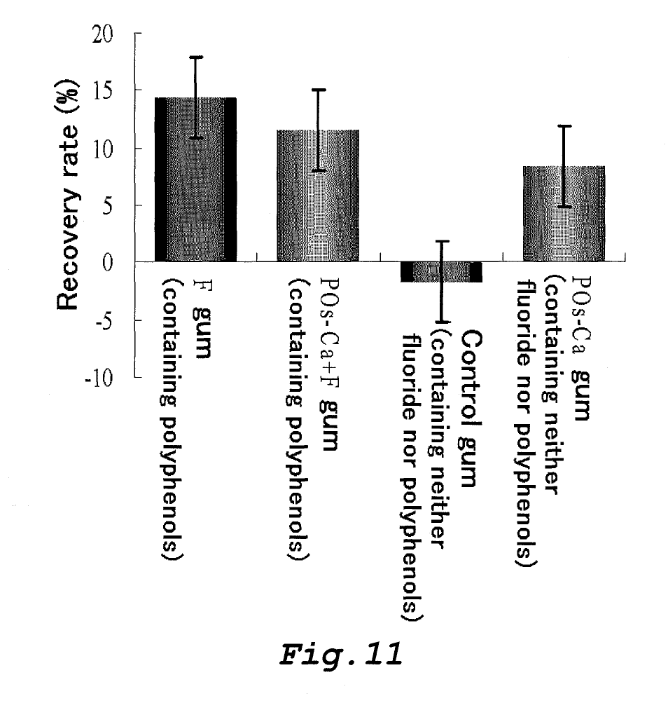

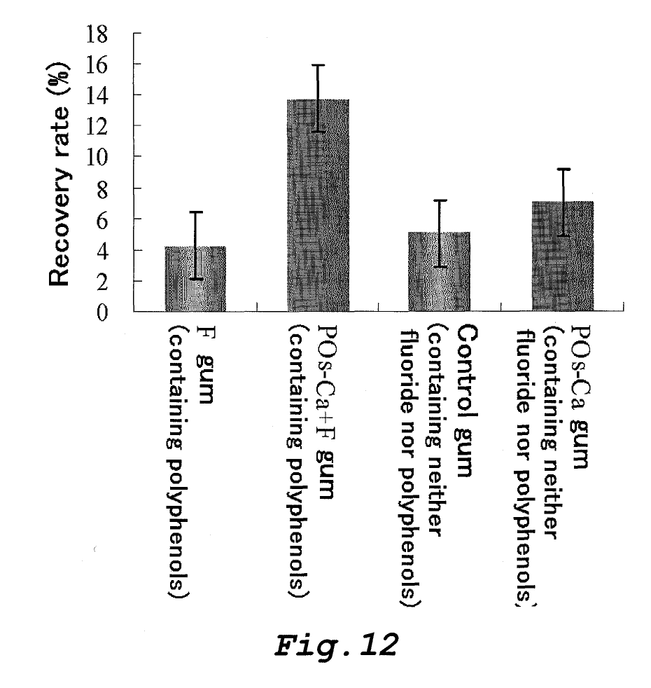

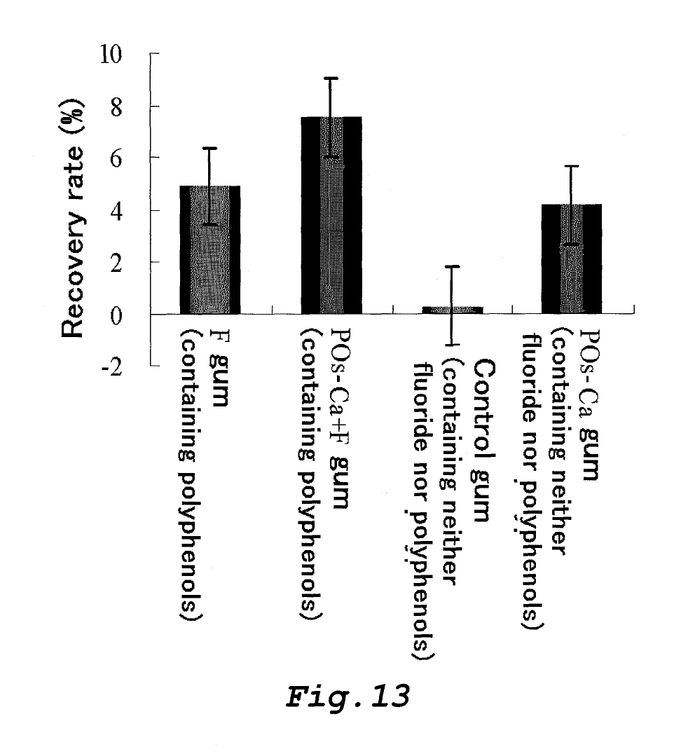

FIG. 10 shows a recovery rate of the amount of mineral loss due to remineralization. FIG. 11 shows a recovery rate of a lesion depth due to remineralization. FIG. 12 shows a recovery rate of the amount of mineral loss after re-demineralization. FIG. 13 shows a recovery rate of a lesion depth after re-demineralization. It can be understood from FIG. 10 that there is little difference in the recovery rate of the amount of mineral loss due to remineralization. However, it can be understood from FIG. 12 that the recovery rate of the amount of mineral loss after demineralization is largely different. It can be understood from FIG. 10 and FIG. 12 that in the case of using a phosphorylated oligosaccharide calcium salt in combination with fluoride, remineralization was excellent. As is apparent from FIG. 11, the recovery rate of a lesion depth after remineralization is the highest for a fluoride-containing gum. However, as is apparent from FIG. 12, the recovery rate of a lesion after demineralization is higher in the case of using a phosphorylated oligosaccharide calcium salt, fluoride and a polyphenol in combination. Therefore, it was found that the recovery rate of a lesion depth is high in the case of using a phosphorylated oligosaccharide calcium salt, fluoride and a polyphenol in combination.

### Example 5

### Production of Candy Containing Phosphorylated oligosaccharide calcium Salt and Tea Extract

In accordance with the method usually carried out in the art, a mixture of palatinit and a reduced starch syrup in a weight ratio of 60:40 was boiled down until the moisture value reaches 1.8% by weight to obtain the candy base. To this candy base, a phosphorylated oligosaccharide calcium salt, a tea extract, a flavor and a coloring agent were added according to the formulation shown in Table 11 below, followed by mixing to prepare a sugarless candy. The weight per candy is about 3.6 g. Assuming that the amount of saliva secreted when one candy was dissolved in the oral cavity was 20 mL, the concentration of the phosphorylated oligosaccharide calcium salt in 20 mL of saliva is about 5.6 mM in terms of the calcium concentration, and an available fluoride concentration is about 0.5 ppm.

TABLE 11
Formulation of sugarless candy

| | | Added amount |
|---|---|---|
| Material | | (% by weight) |
| Candy base | Palatinit | 60 |
| | Reduced starch syrup | 40 |

| | |
|---|---|
| Candy base | 96.7 |
| Phosphorylated oligosaccharide | 2.5 |
| calcium salt*¹ | |
| Tea extract*² | 0.1 |
| Flavor | 0.5 |
| Coloring agent | 0.2 |
| Total | 100.0 |

*¹The phosphorylated oligosaccharide calcium salt was a raw material containing 5% w/w Ca.
*²A material which was obtained by diluting a low polyphenol content-tea extract 2 having a F content of 3,410 ppm and polyphenols content of 9.85%, with distilled water thereby adjusting the F content to 3,000 ppm.

### Example 6

### Production of Tablet Containing Phosphorylated oligosaccharide calcium Salt and Tea Extract

In accordance with the method usually carried out in the art, materials are mixed according to the formulation shown in Table 12 below to prepare tablets. The weight per tablet is about 1 g. Assuming that the amount of saliva secreted when one tablet is dissolved in the oral cavity is 20 mL, the concentration of the phosphorylated oligosaccharide calcium salt in 20 mL of saliva is about 4.6 mM in terms of the calcium concentration, and an available fluoride concentration is about 0.5 ppm.

TABLE 12
Formulation of tablet

| | Added amount |
|---|---|
| Material | (% by weight) |
| Polydextrose | 20 |
| Sugar ester | 2 |
| Phosphorylated oligosaccharide | 7.5 |
| calcium salt*¹ | |
| Tea extract*² | 0.33 |
| Sorbitol | 64.67 |
| Palatinose | 5 |
| Flavor | 0.5 |
| Total | 100.0 |

*¹The phosphorylated oligosaccharide calcium salt is a raw material containing 5% w/w Ca.
*²A material which was obtained by diluting a low polyphenol content-tea extract 2 having a F content of 3,410 ppm and polyphenols content of 9.85%, with distilled water thereby adjusting the F content to 3,000 ppm.

### Example 7

### Production of Tooth Paste Containing Phosphorylated oligosaccharide calcium Salt and Tea Extract

In accordance with the method usually carried out in the art, materials are mixed according to the formulation shown in Table 13 below to produce a tooth paste. Assuming that the amount of saliva secreted when 2 g of this tooth paste is dissolved in the oral cavity is 10 mL during tooth brushing for 5 minutes, the content of the phosphorylated oligosaccharide calcium salt in 10 mL of saliva is about 5 mM in terms of the calcium concentration, and the available fluoride concentration is about 0.5 ppm.

TABLE 13
Formulation of tooth paste

| | Added amount |
|---|---|
| Material | (% by weight) |
| Phosphorylated oligosaccharide | 2 |
| calcium salt*¹ | |
| Tea extract*² | 0.67 |
| Silica | 15 |
| Glycerin | 5 |
| Sorbitol | 20 |
| Sodium carboxymethyl cellulose | 1.5 |
| Sodium lauryl sulfate | 1.5 |
| Saccharin sodium | 0.1 |
| Flavor | 1.0 |
| Sodium benzoate | 0.1 |
| Water | Balance |
| Total | 100.0 |

*¹The phosphorylated oligosaccharide calcium salt is a raw material containing 5% w/w Ca.
*²A material which was obtained by diluting a low polyphenol content-tea extract 2 having a F content of 3,410 ppm and polyphenols content of 9.85%, with distilled water thereby adjusting the F content to 3,000 ppm.

### Example 8

### Production of Mouth Rinsing Agent Containing Phosphorylated oligosaccharide calcium Salt and Tea Extract

In accordance with the method usually carried out in the art, materials are mixed according to the formulation shown in Table 14 below to produce a mouth rinsing agent.

TABLE 14
Formulation of mouth rinsing agent

| | Added amount |
|---|---|
| Material | (% by weight) |
| Phosphorylated oligosaccharide | 5 |
| calcium salt*¹ | |
| Tea extract*² | 0.33 |
| Ethyl alcohol | 5 |
| Sodium lauryl sulfate | 1.5 |
| Glycerin | 10 |
| Menthol | 1.0 |
| Xylitol | 17 |
| Cetylpyridinium chloride | 0.25 |
| Purified water | Balance |
| Total | 100.0 |

*¹The phosphorylated oligosaccharide calcium salt is a raw material containing 5% w/w Ca.
*²A material which was obtained by diluting a low polyphenol content-tea extract 2 having a F content of 3,410 ppm and polyphenols content of 9.85%, with distilled water thereby adjusting the F content to 3,000 ppm.

### Example 9

### Concentration of Fluoride in Saliva in the Co-Existent with Phosphorylated Oligosaccharide

In the case of chewing a food containing a phosphorylated saccharide calcium and a tea polyphenol, it is preferred that the concentration of calcium ions in saliva in the oral cavity is 1 mM to 12 mM, and the concentration of fluoride ions is 0.2 ppm to 100 ppm. Therefore, it was examined to what degree a fluoride can be released as ions in saliva.

The measurements were carried out by the following method. Firstly, each of the three testers were allowed to chew one paraffin pellet (about 1.15 g) for 20 minutes. When the testers feel the accumulation of saliva until 20 minutes after initiation of chewing, the testers were allowed to expel saliva in the oral cavity at any time to obtain chewing saliva. Chewing saliva of the three testers were combined and used. The amount of the chewing saliva was 102.5 ml in total. Next, in I and II, a predetermined amount of tea fluoride or NaF described in Table 15 below and 4 mL of saliva were dispensed in each well. In III and IV, a predetermined amount of tea fluoride or NaF described in Table 15 below and 4 mL of a solution prepared by dissolving POs-Ca in a required amount of saliva were dispensed in each well. Under this condition, POs-Ca was added in an amount per chewing of one POs-Ca-containing center gum. Next, the pH was measured by an ion electrode, the concentration of fluoride ions was measured by an electrode, the concentration of calcium ions, and the concentration of phosphate ions were measured by an electrode.

TABLE 15

| | | | | | Results | | | | |
|---|---|---|---|---|---|---|---|---|---|
| | | | | | | | | Amount of | |
| | | | | | | | | F added | |
| | | | | | | | Measured | (F | |
| | Composition of solution | | | | | Measured | value-control | theoretical | Recovery |
| | Saliva | Tea F | NaF | POs-Ca | | value of F | value | value) | rate of F |
| No. | (mL) | (μL) | (μL) | (mg) | pH | (ppm) | (ppm) | (ppm) | (%) |
| I-1 | 4 | 20 | — | — | 7.63 | 0.541 | 0.472 | 0.5 | 94.4 |
| I-2 | 4 | 40 | — | — | 7.55 | 0.908 | 0.839 | 1.0 | 83.9 |
| I-3 | 4 | 60 | — | — | 7.52 | 1.310 | 1.241 | 1.5 | 82.7 |
| II-1 | 4 | — | 20 | — | 7.65 | 0.556 | 0.487 | 0.5 | 97.4 |
| II-2 | 4 | — | 40 | — | 7.68 | 0.983 | 0.914 | 1.0 | 91.4 |
| II-3 | 4 | — | 60 | — | 7.73 | 1.360 | 1.291 | 1.5 | 86.1 |
| III-1 | 3 | 15 | — | 4 | 7.24 | 0.351 | 0.282 | 0.5 | 56.4 |
| III-2 | 3 | 30 | — | 4 | 7.19 | 0.653 | 0.584 | 1.0 | 58.4 |
| III-3 | 3 | 45 | — | 4 | 7.16 | 1.050 | 0.981 | 1.5 | 65.4 |
| IV-1 | 3 | — | 15 | 4 | 7.43 | 0.390 | 0.321 | 0.5 | 64.2 |
| IV-2 | 3 | — | 30 | 4 | 7.40 | 0.820 | 0.751 | 1.0 | 75.1 |
| IV-3 | 3 | — | 45 | 4 | 7.40 | 1.190 | 1.121 | 1.5 | 74.7 |
| V | 3 | — | — | 4 | 7.37 | 0.0691 | — | — | — |

V = Control
Tea fluoride (tea F) and NaF were added to each solution in the amount described in the table after adjusting a solution so that the concentration of a fluoride was 100 ppm.

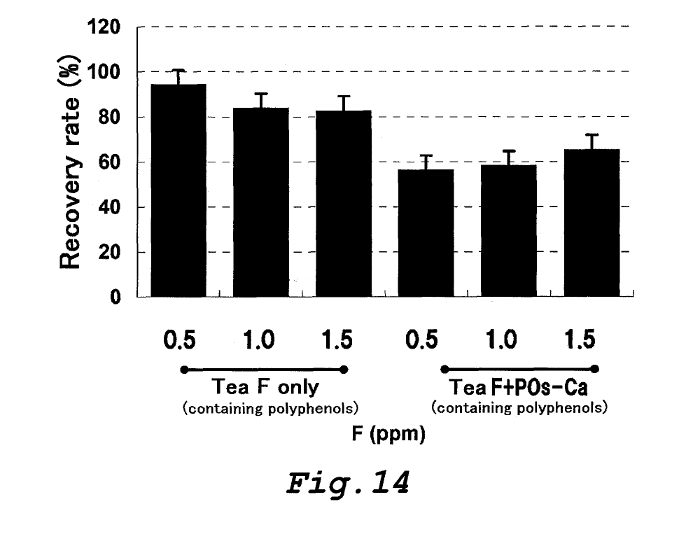

A recovery rate in the case of only tea fluoride and the results in the case of containing tea fluoride and POs-Ca are shown in FIG. 14. When POs-Ca and tea fluoride are dissolved in saliva, fluoride eluted from the tea fluoride accounts for about 50% to about 60% based on the added concentration. Therefore, it can be understood only about half of the added amount was eluted. Therefore, it is considered to be desirable that the tea fluoride is added in the amount of about 2 times the amount of the objective elution concentration in the case of using POs-Ca in combination with the tea fluoride.

### Example 10 Design of AddedAmount and Actual Elution Amount

Taking the results of Example 9 into consideration, the added amount was calculated. The calculation was carried out in the following manner:

POs-Ca is added in 3.7% per 1 g of one center gum. The weight of calcium is 5% by weight of the weight of POs-Ca. The amount of the gum used per time is two gum pellets. The amount of saliva is about 20 mL on average. Therefore, the amount of calcium in these two gum pellets is as follows: 1 g×3.7%×5%×two gum pellets=1.85×2 mg=3.7 mg. Considering that almost all of the calcium is eluted in 20 mL of saliva, since the molecular weight of calcium is about 40, the concentration of calcium in the saliva is as follows: 3.7 mg/(20 mL×40)≈4.6×10⁻³ M=4.6 mM.

Next, the amount of fluoride is calculated. When 0.2% by weight of a tea extract having the fluoride content of 3,000 ppm is added with raw materials of two gum granules, the fluoride concentration is 0.58 ppm. Since about 50 to 60% of fluoride is eluted in saliva as described in Example 9, the calculation is carried out assuming that the 60% of fluoride is eluted. It is considered to elute 0.5 ppm or more. When 0.4% by weight of the tea extract is added with the gum, the following results are obtained: 0.58×2=1.16 ppm in the case of the entire elution. Considering that 60% is eluted, the concentration in the saliva is as follow: 1.16 ppm×60%=0.696 ppm. Thereby, the concentration in the saliva can be made to be 0.5 ppm or more.

The composition of a center gum portion of the gum thus designed is shown below.

TABLE 16
Composition of center gum

| | | Comparative |
|---|---|---|
| | Example 10 | Example 10 |
| | POs-Ca + F | POs-Ca(−)F(−) |
| | Added amount | Added amount |
| Material | (% by weight) | (% by weight) |
| Gum base | 35 | 35 |
| POs-Ca*¹ | 3.7 | 0 |
| Low polyphenol | 0.4 | 0 |
| content-tea extract*² | | |
| Xylitol | 56.9 | 61 |
| Glycerin | 1 | 1 |
| Mint oil | 3 | 3 |
| Total | 100.0 | 100.0 |

*¹Raw material containing 5% w/w Ca; powder.
*²A material which was obtained by diluting a low polyphenol content-tea extract 2 having a F content of 3,410 ppm and polyphenols content of 9.85%, with distilled water thereby adjusting the F content to 3,000 ppm.

In accordance with the method usually carried out in the art, materials according to the formulation described in Table 16 were used as materials of a center gum and conventionally sugar coated to produce a pellet gum. The gum of Example 10 was a gum containing POs-Ca+F, and the gum of Comparative Example 10 was a control gum containing neither POs-Ca nor F. The weight per pellet gum was about 1.5 g on average, the weight of the center gum portion was about 1.0 g on average, and the weight of the sugar coating portion was about 0.5 g on average.

For each gum, two subjects were allowed to chew the gum for 20 minutes. When the subjects feel the accumulation of saliva during 20 minutes, the subjects were allowed to expel saliva, and saliva was separately collected every 5 minutes. In this case, the subjects were prohibited to spit out the gum. The concentration of calcium ions, the concentration of phosphoric acid and the concentration of fluoride ions in each saliva sample were measured by an electrode method. The measurement results of POs-Ca+F/gum are shown in Table 17 below.

TABLE 17
Gum of POs-Ca + F (added 0.4% of a low polyphenol content-tea extract) n = 2

| Time | Amount of | | | Ca | P | F | F | Ca | P |
|---|---|---|---|---|---|---|---|---|---|
| (minutes) | Saliva (mL) | pH | Ca/P | (mM) | (mM) | (μg/l) | (ppm) | (mg) | (mg) |
| 5 | 18.8 | 7.15 | 1.91 | 4.46 | 2.34 | 890.50 | 0.89 | 3.12 | 1.64 |
| 10 | 11.8 | 7.31 | 0.95 | 2.25 | 2.37 | 366.50 | 0.37 | 1.12 | 1.18 |
| 15 | 10.5 | 7.34 | 0.68 | 1.78 | 2.59 | 242.00 | 0.24 | 0.78 | 1.14 |
| 20 | 10.0 | 7.37 | 0.54 | 1.50 | 2.78 | 199.50 | 0.20 | 0.60 | 1.11 |

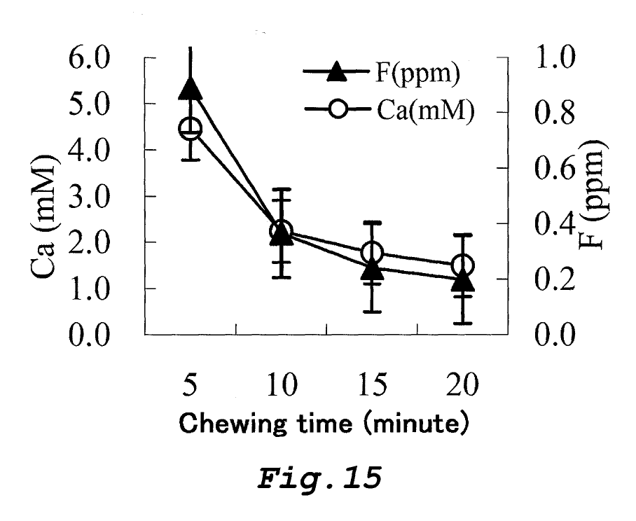

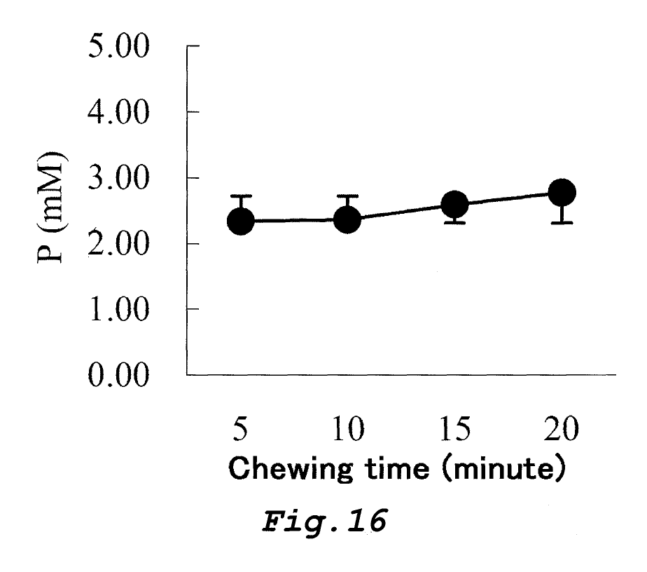

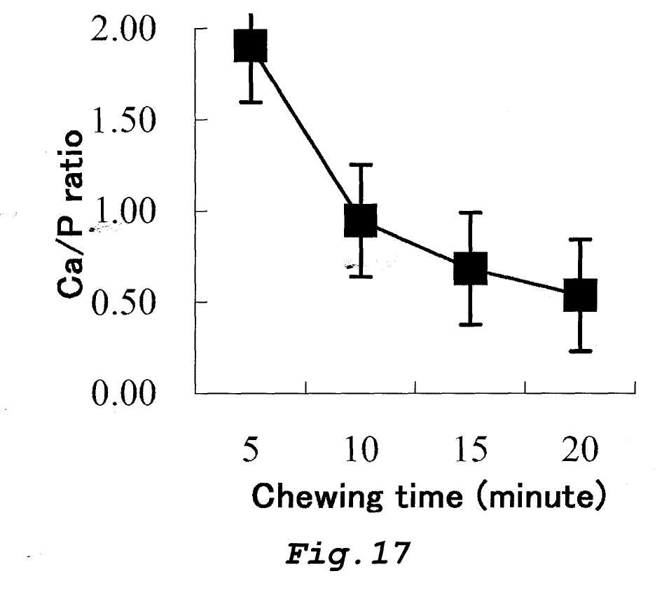

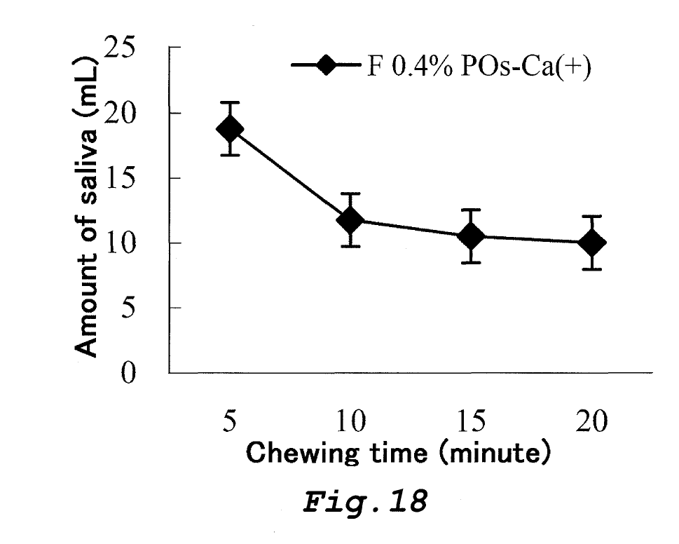

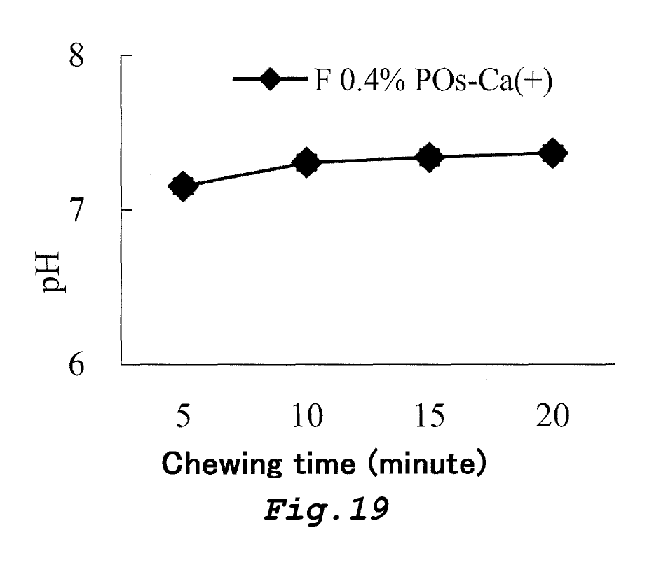

The results are shown in FIGS. 15 to 19. As a result, in the gum for POs-Ca+F, firstly, high-concentration calcium and fluoride were eluted and then the elution amount gradually decreased. On the other hand, in the gums of Comparative Examples, since neither calcium nor fluoride were mixed, the amounts of fluoride and calcium in saliva hardly vary and were nearly constant.

The total amount of fluoride ions eluted in saliva can be determined by multiplying the amount of saliva by the concentration of fluoride ions, and summing up the obtained products. Total=18.8×0.89+11.8×0.37+10.5×0.24+10.0×0.20≈23.5. The total is divided by the amount of saliva to obtain an average concentration of fluoride ions.

Average=23.5/(18.8+11.8+10.5+10.0)≈0.5(ppm)

In this case, since the measurement was carried out on expelled saliva, the concentration of fluoride ions and the concentration of calcium ions in saliva gradually decreased. However, it is considered that chewing is not carried out while expelling saliva in the case of usually chewing the gum, and thus the concentration of fluoride ions and the concentration of calcium ion in the oral cavity do not largely decrease. Therefore, it is considered that about 0.5 ppm of the concentration of fluoride ions can be achieved for about 20 minutes in the oral cavity.

### Example 11 and Comparative Example 11

### Production of Gum and Prevention of Tooth Erosion due to the Gum

### (1) Production of Gum

In accordance with the method usually carried out in the art, materials according to the formulation described in Table 18 below were used as materials of a center gum and conventionally sugar coated to produce pellet gums. The gum of Example 11 is a gum with a formulation of 2.5% of POs-Ca+1.2% of a high fluoride content-tea extract (POs-Ca+F (containing polyphenol)), the gum of Comparative Example 11-1 is a gum containing no POs-Ca and with a formulation of 1.2% of a high fluoride content-tea extract (F(containing polyphenol)), the gum of Comparative Example 11-2 is a gum with a formulation of 2.5% POs-Ca (POs-Ca) which contains no tea extract, and the gum of Comparative Example 11-3 was a POs-Ca-non-added gum (control) containing neither POs-Ca nor a tea extract. The weight per pellet gum was about 1.5 g on average, the weight of the center gum portion was about 1.0 g on average, and the weight of the sugar coating portion was about 0.5 g on average.

TABLE 18
Formulation of center gum

| | Example 11 | Comparative | | Comparative |
|---|---|---|---|---|
| | POs-Ca + F | Example 11-1 | Comparative | Example 11-3 |
| | (containing | F (containing | Example 11-2 | Control |
| | polyphenol) | polyphenol) | POs-Ca | (POs-Ca(−)F(−)) |
| | Added amount | Added amount | Added amount | Added amount |
| Material | (% by weight) | (% by weight) | (% by weight) | (% by weight) |
| Gum base | 35 | 35 | 35 | 35 |
| POs-Ca*¹ | 3.7 | 0 | 3.7 | 0 |
| Low polyphenol | 1.2 | 1.2 | 0 | 0 |
| content-tea | | | | |
| extract*² | | | | |
| Xylitol | 56.1 | 59.8 | 57.3 | 61.0 |
| Glycerin | 1 | 1 | 1 | 1 |
| Mint oil | 3 | 3 | 3 | 3 |
| Total | 100.0 | 100.0 | 100.0 | 100.0 |

*¹Raw material containing 5% w/w Ca; powder.
*²A material which was obtained by diluting a low polyphenol content-tea extract 2 having a F content of 3,410 ppm and polyphenols content of 9.85%, 34.1 folds with distilled water thereby adjusting the F content to 100 ppm and the polyphenol content to 0.289%.

(2) Tooth Erosion Prevention Test

Tooth pieces to be used: In accordance with the method described in “3. Formation of Subsurface Demineralized Lesion”, bovine tooth pieces were prepared. A test of 5 tooth pieces was replicated three times per 1 test treatment. 60 tooth pieces in total were used in 4 test treatments.

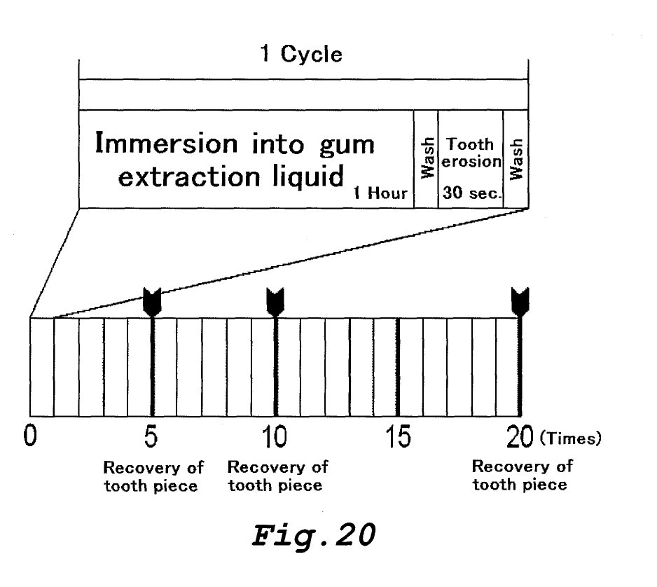

In accordance with the following test method, a tooth erosion prevention test was carried out. A schematic diagram of the test cycle is shown in FIG. 20.

Test method
1. A water bath was heated to 37° C. in advance.
2. A window of 3 mm×3 mm was formed at a central portion of a tooth piece using a nail varnish.
3. 600 ml of artificial saliva (the composition is shown in Table 19) and citric acid/artificial saliva solution (pH 3.0) (the composition is shown in Table 20) were prepared and subsequently these solutions were incubated until the temperatures of said solutions reach 37° C.
4. 30 replicate gum pellets for each were squeezed repeatedly in 300 ml of incubated artificial saliva at 37° C. for 20 minutes (in a warm bath at 37° C.).
5. Tooth pieces were immersed in the solution prepared by squeezing the gum, and then incubated in a warm bath at 37° C. for 1 hour.
6. The tooth piece was recovered and washed with distilled water.
7. The tooth piece was immersed in a citric acid/artificial saliva solution (pH 3.0) (the composition is shown in Table 20) for 30 seconds.
8. The tooth piece was recovered and washed with distilled water.
9. One cycle consisting of steps 5 to 8 was repeated 20 times.
10. The tooth pieces subjected to the 5 cycles, 10 cycles and 20 cycles were immersed in distilled water and an acid was extracted overnight, and then CLSM profiles were measured by CLSM and surface roughness (roughness) were measured by Surfcorder SE500.

TABLE 19
Composition of artificial saliva

| | |
|---|---|
| distilled water | 50 ml |
| 2M KCl | 5.0 ml |
| 1M HCl | 0.05 ml |
| 100 mM KH₂PO₄ | 3.6 ml |
| 100 mM CaCl₂ | 1.5 ml |
| 200 mM HEPES | 10 ml |

After adjusting to pH 6.5 with HCl, distilled water was added
to make up to a total of 100 ml.

| | |
|---|---|
| Total | 100 ml |

TABLE 20
Composition of citric acid/artificial saliva (pH 3.0)

| | |
|---|---|
| distilled water | 40 ml |
| 2M KCl | 5.0 ml |
| 1M HCl | 0.05 ml |
| 100 mM KH₂PO₄ | 3.6 ml |
| 100 mM CaCl₂ | 1.5 ml |
| 200 mM HEPES | 10 ml |

After adjusting to pH 3.0 with 2% citric acid, distilled
water was added to make up to a total of 100 ml.

| | |
|---|---|
| Total | 100 ml |

TABLE 21
Measuring Conditions

CLSM
(Ultra-depth shape measuring microscope: VK-8510, KEYENCE)
Objective lend: 50 times magnification
PITCH: 0.2 μm
RUNMODE: Color ultra-depth
OPTICAL ZOOM: 1x
Surfcorder SE500
(Surface roughness analyzer, Kosaka Laboratory Ltd.)
Measuring magnification: x2,000
Feed speed: 0.5 mm/s
Trace length: 8,800 mm
Cut-off: λc 0.8 mm
Evaluation length: Cut-off × 10

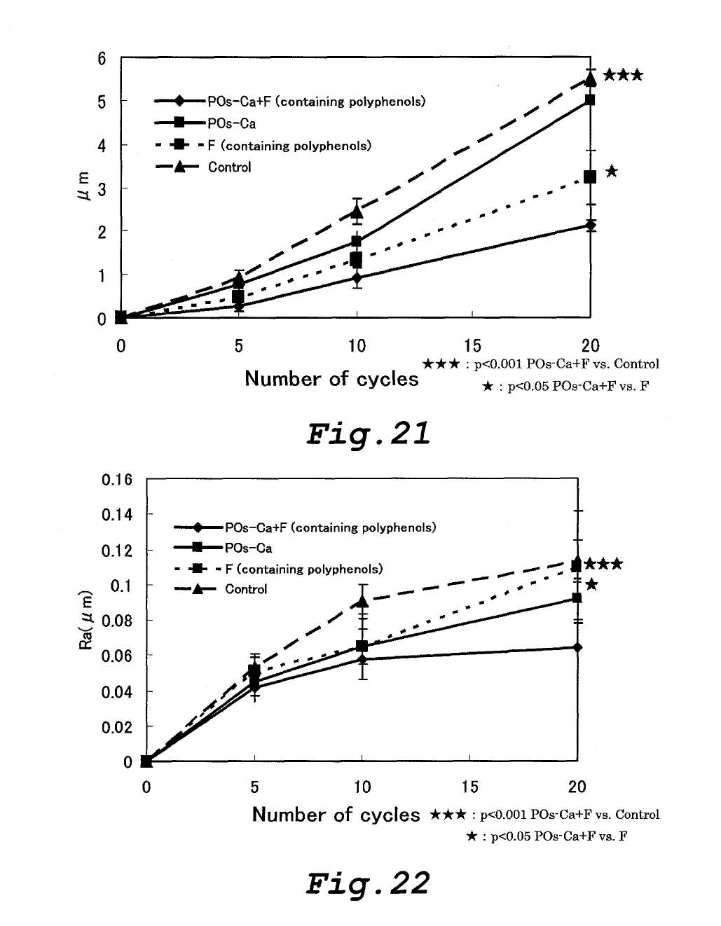

The results of CLSM profile are shown in FIG. 21. As the numerical value of CLSM profile is lower, it is indicated that the deeper portion of the tooth is packed more densely. Therefore, it is confirmed that the tooth is packed most densely in the case of POs-Ca+F (containing polyphenol).

The results of surface roughness profile are shown in FIG. 22. As the surface roughness profile is larger, it indicates that the surface of teeth is rougher. Therefore, it is confirmed that the surface of tooth is the smoothest in the case of POs-Ca+F (containing polyphenol).

As described above, it is confirmed that the highest effect of preventing dental caries due to an acid is exerted in the case of POs-Ca+F (containing polyphenol) (i.e., in the case of containing POs-Ca, tea fluoride and a tea polyphenol).

### Example 12

### Combination of Fluoride Agent Other than Tea Fluoride, POs-Ca and Polyphenol

In order to evaluate the influence of a combination of a fluoride agent other than tea fluoride, POs-Ca and a polyphenol on mineralization, a change of the pH and calcium ions over time in various remineralization solutions was examined.

Specifically, remineralization solutions with the compositions of Example 12-1, 12-2 or 12-3 in Table 22 shown below were prepared. In the remineralization solution of Example 12-1, strontium fluoride is used as the fluoride agent. In the remineralization solution of Example 12-2, sodium monofluorophosphate is used as the fluoride agent. In the remineralization solution of Example 12-3, potassium fluoride is used as the fluoride agent. All of these solutions contained a phosphoric acid source compound (KH₂PO₄) and a low polyphenol content-tea extract. All of the supply sources of calcium were a phosphorylated oligosaccharide calcium salt.

TABLE 22
Composition of each remineralization solution

| | Example 12-1 | | Example 12-2 | | Example 12-3 | |
|---|---|---|---|---|---|---|
| | Final | Added | Final | Added | Final | Added |
| | concentration | amount | concentration | amount | concentration | amount |
| Composition | (mM) | (ml) | (mM) | (ml) | (mM) | (ml) |
| Distilled water | — | 50 | — | 50 | — | 50 |
| 2M KCl solution | 100 | 5.0 | 100 | 5.0 | 100 | 5.0 |
| 1M HCl solution | — | 0.05 | — | 0.05 | — | 0.05 |
| 100 mM KH₂PO₄ | 3.6 | 3.6 | 3.6 | 3.6 | 3.6 | 3.6 |
| solution | | | | | | |
| 10% POs-Ca | 6.25 | 5.0 | 6.25 | 5.0 | 6.25 | 5.0 |
| solution*¹ | | | | | | |
| Polyphenol | 0.001% | 0.1 g | 0.001% | 0.1 g | 0.001% | 0.1 g |
| powder*² | by weight | | by weight | | by weight | |
| SrF₂ solution*³ | 10 ppm | 1.0 | — | — | — | — |
| SMFP solution*⁴ | — | — | 10 ppm | 1.0 | — | — |
| KF solution*⁵ | — | — | — | — | 10 ppm | 1.0 |
| 200 mM HEPES(pH | 20 | 10 | 20 | 10 | 20 | 10 |
| 7.0) | | | | | | |
| Distilled water | After adjusting to pH 6.5, distilled water was | | | | | |
| and 1M HCl | added so that the total amount is 100 ml. | | | | | |
| solution or 1M | | | | | | |
| KOH solution | | | | | | |
| Total | — | 100 | — | 100 | — | 100 |
| Final fluoride | 10 ppm | | 10 ppm | | 10 ppm | |
| content | | | | | | |

*¹Solution containing 5% w/w Ca.
*²“Polyphenon 70A” manufactured by Mitsui Norin Co., Ltd.; polyphenol content >98%.
*³SrF₂ solution = strontium fluoride; solution containing 1,000 ppm of F.
*⁴SMFP solution = sodium monofluorophosphate solution; solution containing 1,000 ppm of F.
*⁵KF solution = potassium fluoride solution; solution containing 1,000 ppm of F.

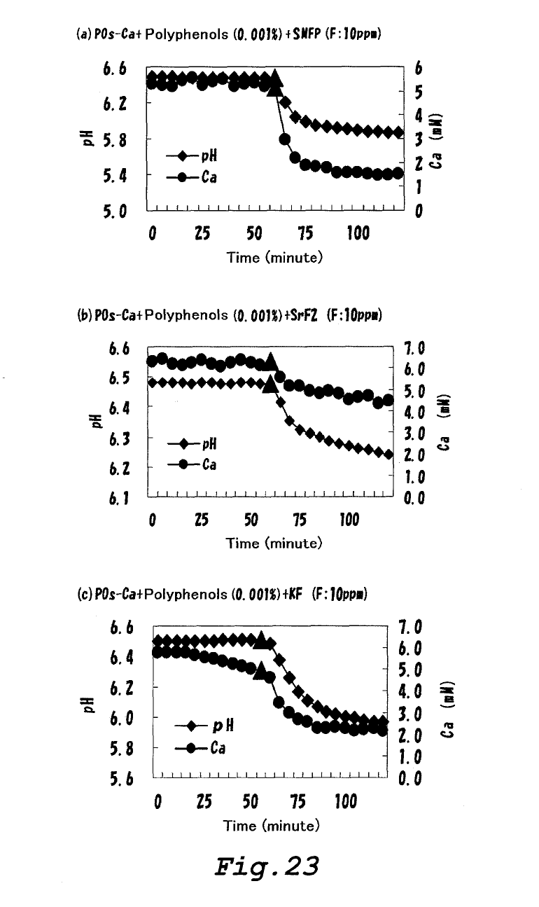

Immediately after preparing these solutions, incubation was initiated at 37° C. at pH 6.5. During incubation, change in pH and Ca concentration were measured every 5 minutes. The pH and Ca concentration at each time point were measured by an electrode. After initiation of the incubation, crystal nuclei (100 mg of hydroxyapatite (manufactured by Wako Pure Chemical Industries, Ltd.)) was added to each solution at a time point of 60 minutes for all of Examples 12-1, Example 12-2 and Example 12-3, and then the incubation and the measurements were continued until a time point of 120 minutes. The results of Example 12-1 are shown in FIG. 23 (a), the results of Example 12-2 are shown in FIG. 23 (b), and the results of Example 12-3 are shown in FIG. 23 (c).

As a result, it is confirmed that solubility of both calcium ions and fluoride ions can be maintained in a neutral range even when a fluoride agent (strontium fluoride=SrF₂, monofluorophosphoric acid=MFP) conventionally used in pharmaceuticals, quasi-drugs and dental materials is used in combination with POs-Ca, and further, in a remineralized site, both ions can be supplied to the remineralized site.

### Example 13 and Comparative Examples 13-1 to 13-4

### Improvement of Quality of Taste of POs-Ca by Low Polyphenol Content-Tea Extract

### (1) Production of Gum

A 10% POs-Ca solution (a solution containing 5% w/w Ca), low polyphenol content-tea extract (a material which was obtained by diluting a low polyphenol content-tea extract 2 having a F content of 3,410 ppm and polyphenols content of 9.85%, 34.1 folds with distilled water thereby adjusting the F content to 100 ppm and the polyphenol content to 0.289%) and a polyphenol powder (“Polyphenon 70A”, manufactured by Mitsui Norin Co., Ltd.; polyphenol content >98%) were diluted with distilled water to prepare 20 mL of the following aqueous solutions.

### Example 13-1

POs-Ca+F (0.5 ppm)

### Comparative Example 13-1

POs-Ca+F (0.5 ppm)+Polyphenols (1.0%)

### Comparative Example 13-2

POs-Ca+F (0.5 ppm)+Polyphenols (0.1%)

### Comparative Example 13-3

POs-Ca+F (0.5 ppm)+Polyphenols (0.05%)

### Comparative Example 13-4

POs-Ca+F (5.0 ppm)

### Comparative Example 13-5

POs-Ca only.

Wherein, “POs-Ca” indicates to contain 2.5% by weight of POs-Ca, “F (0.5 ppm)” indicates to contain 0.5 ppm of fluoride derived from a low polyphenol content-tea extract, and “polyphenol (1.0%)” indicates to contain 1.0% by weight of a polyphenol powder.

The aqueous solution of Example 13-1 is an aqueous solution added POs-Ca and a low polyphenol content-tea extract (POs-Ca+tea fluoride (containing low concentration polyphenols)), the aqueous solutions of Comparative Examples 13-1 to 13-3 are aqueous solutions added, in addition to POs-Ca and a low polyphenol content-tea extract, polyphenols (POs-Ca+tea fluoride+high concentration polyphenol), and the aqueous solution of Comparative Example 13-4 is an aqueous solution added POs-Ca and a high fluoride content-tea extract (POs-Ca+tea fluoride (containing a low concentration polyphenol)). The aqueous solutions of Comparative Examples 13-1 to 13-3 are solutions based on the assumption of tea extracts of prior art. The aqueous solution of Comparative Example 13-5 is an aqueous solution added POs-Ca only.

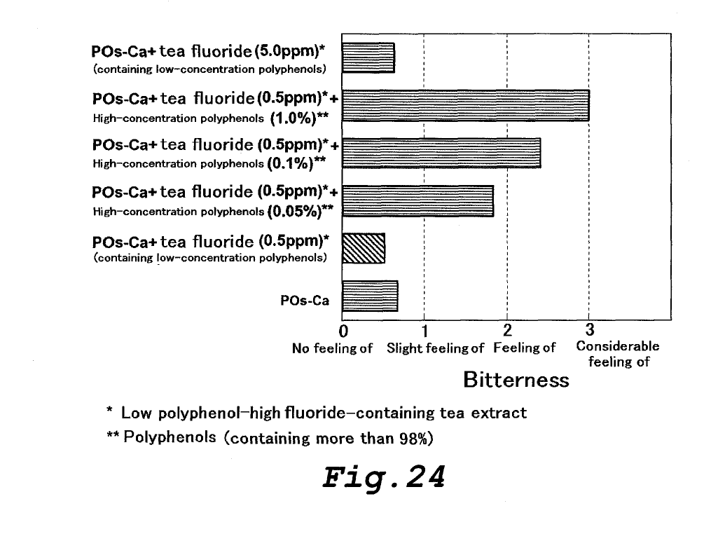

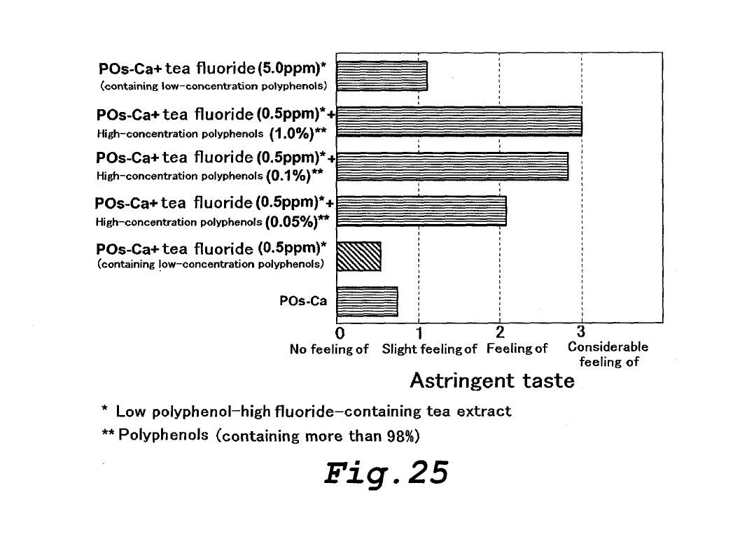

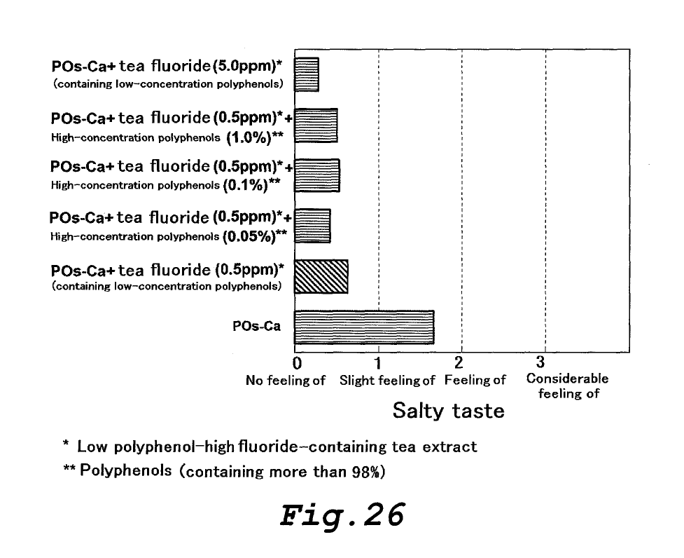

Tastes of the aqueous solutions prepared were evaluated by six expert testers. The testers were allowed to intake the aqueous solutions and to evaluate whether to feel bitterness, astringent taste and salty taste. Each test was carried out by the following evaluation criteria. No feeling of bitterness=0, slight feeling of bitterness 1, feeling of bitterness=2, considerable feeling of bitterness=3; no feeling of astringent taste=0, slight feeling of astringent taste=1, feeling of astringent taste=2, considerable feeling of astringent taste=3; no feeling of salty taste=0, slight feeling of salty taste=1, feeling of salty taste=2, and considerable feeling of salty taste=3. The obtained evaluation scores were averaged. The results of bitterness are shown in FIG. 24, the results of astringent taste are shown in FIG. 25, and the results of salty taste are shown in FIG. 26.

As apparent from FIG. 24, the testers experienced strong bitterness from the gum containing a large amount of polyphenols, whereas, the testers hardly had the feeling of bitterness from gum containing only a small amount of polyphenols. Even in the case where a large amount of a low polyphenol-high fluoride content-tea extract was added so that the fluoride concentration was 5.0 ppm, the testers hardly had the feeling of bitterness. This reveals that the taste of the gum does not deteriorate even when a large amount of the low polyphenol-high fluoride content-tea extract is added.

As is apparent from FIG. 25, the testers had the feeling of strong astringent taste from gum containing a large amount of polyphenols, whereas, the testers hardly had the feeling of astringent taste from gum containing only a small amount of polyphenols. Even in the case where a large amount of a low polyphenol-high fluoride content-tea extract was added so that the fluoride concentration was 5.0 ppm, the testers hardly had the feeling of astringent taste. This reveals that taste of the gum does not deteriorate even when a large amount of the low polyphenol-high fluoride content-tea extract is added.

As apparent from FIG. 26, the testers hardly had the feeling of saltiness from gum containing a low polyphenol-high fluoride content-tea extract, whereas, the testers had the feeling of saltiness from gum containing no low polyphenol-high fluoride content-tea extract and containing POs-Ca. This reveals that saltiness of POs-Ca can be relieved by formulating the low polyphenol-high fluoride content-tea extract.

As a result, it was found that bitterness and astringent taste generated by the addition of a conventional tea extract can be improved by using a tea extract having a decreased polyphenol content. It was also found that bitterness and saltiness of conventional POs-Ca can be improved by adding a small amount of polyphenol using a tea extract with a decreased polyphenol content.

As described above, the present invention has been exemplified using a preferred embodiment of the present invention, but the present invention should not be construed to be limited to this embodiment. It is understood that the present invention should be construed for its scope only by the claims. It is understood that those skilled in the art can practice an equivalent range based on the description of the invention and the technical common knowledge, from the description of the specific preferable embodiment of the present invention. It is understood that patents, patent applications and publications cited in the present specification are herein incorporated by reference for the content thereof as if the contents themselves were specifically described in the present specification.

### INDUSTRIAL APPLICABILITY

According to the present invention, a food and an oral composition, capable of obtaining a remineralization effect which is excellent as compared to those of prior art, are provided.

By using POs-Ca in combination with fluoride, insolubilization due to reaction of fluoride ions with calcium ions is prevented, thus making it possible to efficiently supply calcium ions and fluoride ions to the target tooth surface.

When the content of polyphenol which inhibits remineralization is decreased, applicability of a tea-derived fluoride is enhanced.

1. A cariostatic food comprising:
(1) (i) a phosphorylated saccharide calcium salt; or
(ii) a combination of
a phosphorylated saccharide salt other than a phosphorylated saccharide calcium salt or
a phosphorylated saccharide, and
a water-soluble calcium salt other than a phosphorylated saccharide calcium salt;
(2) a fluoride; and
(3) a polyphenol;
wherein
the phosphorylated saccharide is composed of a saccharide moiety and phosphate group(s);
the content of the component (1) in the food is in an amount suitable to make the calcium concentration in saliva in the oral cavity to be 1 mM to 12 mM when the food exists in the oral cavity;
the content of the fluoride in the food is in an amount suitable to make the fluoride concentration in saliva in the oral cavity to be 0.2 ppm to 100 ppm when the food exists in the oral cavity;
the content of the polyphenol in the food is in an amount suitable to make the polyphenol concentration in saliva in the oral cavity to be 0.001% by weight to 0.1% by weight when the food exists in the oral cavity; and
the food remains in the oral cavity for 5 minutes or more upon eating.

2.-3. (canceled)

4. The food according to claim 1, which is a chewing gum, a candy, tablet candy or a frozen dessert.

5. The food according to claim 1, wherein the polyphenol is a tea polyphenol.

6. The food according to claim 1, wherein the saccharide moiety is a glucan or a reduced glucan.

7. The food according to claim 6, wherein the degree of polymerization of the saccharide moiety is 2 to 8.

8. The food according to claim 7, wherein the number of the phosphate group(s) is from 1 to 2.

9. The food according to claim 1, wherein the component (1) is a phosphorylated saccharide calcium salt.

10. The food according to claim 1, which further contains a phosphoric acid source compound.

11. The food according to claim 10, wherein the phosphoric acid source compound is selected from the group consisting of phosphoric acid, sodium phosphate, potassium phosphate, polyphosphoric acid and a cyclic phosphate.

12. The food according to claim 10, wherein the content of the phosphoric acid source compound in the food is in an amount suitable to make the phosphoric acid concentration in saliva in the oral cavity to be 9 mM or less when the food exists in the oral cavity.

13. The food according to claim 1, wherein the content of the polyphenol in the food is in an amount suitable to make the polyphenol amount in saliva in the oral cavity to be 0.001% by weight to 0.02% by weight when the food exists in the oral cavity.

14. The food according to claim 1, wherein the content of the fluoride in the food is in an amount suitable to make the fluoride concentration in saliva in the oral cavity to be 0.2 ppm to 1 ppm when the food exists in the oral cavity.

15.-33. (canceled)
